(19) United States
(12) Patent Application Publication
(10) Pub. No.: US 2009/0325956 A1
(43) Pub. Date: Dec. 31, 2009

(54) Aromatic amine derivative and use thereof

(76) Inventors: Takahiko Taniguchi, Osaka (JP); Kenichi Miyata, Ibaraki (JP); Osamu Kubo, Osaka (JP); Junji Matsui, Osaka (JP)

Correspondence Address: WENDEROTH, LIND & PONACK, L.L.P., 1030 15th Street, N.W.,, Suite 400 East, Washington, DC, 20005-1503, US

(21) Appl. No.: 12/311,776

(22) PCT Filed: Oct. 12, 2007
(86) PCT No.: PCT/JP2007/069962

§ 371 Date: Apr. 13, 2009

(30) Foreign Application Priority Data
Oct. 13, 2006 (JP) 2006-280625

(51) Int. Cl. A61K 31/5377; A61P 3/04; A61P 3/06; C07D 413/04

(52) U.S. Cl. 514/235.5; 544/124

(57) ABSTRACT

The present invention provides a novel SCD inhibitor. An SCD inhibitor containing a compound represented by the formula [I]

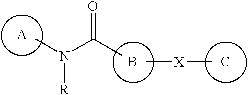

wherein ring A is an optionally substituted aromatic ring, ring B is an optionally substituted ring, ring C is an optionally substituted aromatic ring, R is a hydrogen atom, an optionally substituted hydrocarbon group or an optionally substituted heterocyclic group, and X is a spacer having 1 to 5 atoms in the main chain, or a salt thereof, or a prodrug thereof.

### TECHNICAL FIELD

The present invention relates to a novel compound having a Stearoyl-CoA desaturase (hereinafter sometimes to be abbreviated as SCD) inhibitory action. Moreover, the present invention relates to an agent for the prophylaxis or treatment of hyperlipidemia, diabetes, obesity, lipid metabolism abnormality, fatty liver, metabolic syndrome, arteriosclerosis associated disease, cardiovascular disease and the like, which comprises a compound having an SCD inhibitory action or a salt thereof or a prodrug thereof.

### BACKGROUND ART

Being one of the enzymes localized in endoplasmic reticulum, SCD is a rate determining enzyme of monounsaturated fatty acid synthesis, and introduces a double bond into the position of Δ9 of saturated fatty acid. SCD has selectivity for palmitic acid and stearic acid, and converts them to palmitoleic acid and oleic acid (J Biol Chem. 1976 Aug. 25; 251(16): 5095-103; Prog Lipid Res. 1995; 34(2): 139-50). The products resulting from these enzyme reactions are most abundantly contained in various fats such as phospholipid, triglyceride, cholesterol ester, wax ester and the like (Prostaglandins Leukot Essent Fatty Acids. 1995 October; 53(4): 279-86; J Lipid Res. 2002 December; 43(12): 2146-54). In addition, monounsaturated fatty acid is not only a constituent factor of fat but also plays an important role as a mediator of intercellular signaling, cell differentiation, apoptosis and the like (Dev Neurosci. 1992; 14(1): 61-8; FEBS Lett. 1999 Jul. 2; 454(1-2): 42-6; J Lipid Res. 1999 September; 40(9): 1549-58; Diabetes. 1999 October; 48(10): 2007-14; Immunology. 2002 December; 107(4): 435-43; Proc Natl Acad Sci USA. 2003 Mar. 18; 100(6): 3077-82). Since monovalent unsaturated fatty acid have a wide variety of functions, variation in the SCD activity is considered to possibly influence various metabolic pathways relating to diabetes, obesity, abnormal lipid metabolism, fatty liver, metabolic syndrome, arteriosclerosis associated disease and cardiovascular disease.

As SCD genes, two types (SCD1, SCD2) in rat (GenBank ACCESSION No.: NM_{—}139192; GenBank ACCESSION No.: NM_{—}031841), and four types in mouse (SCDs 1, 2, 3, and 4) (GenBank ACCESSION No.: NM_{—}009127; GenBank ACCESSION No.: NM_{—}009128; GenBank ACCESSION No.: NM_{—}024450; GenBank ACCESSION No.: NM_{—}183216) are cloned. SCD1 is expressed in various tissues, and characteristically under control of diet and hormone factors including insulin, cholesterol and polyvalent unsaturated fatty acid (Curr Opin Lipidol. 2003 June; 14(3): 255-61). In human, two types (SCD1 and SCD5) of genes have been cloned (GenBank ACCESSION No.: NM_{—}005063; GenBank ACCESSION No.: NM_{—}001037582), and the identity of SCD1 amino acid sequence between mouse and human is as high as 85% (Biochem J. 1999 May 15; 340 (Pt 1): 255-64; Gene. 2003 Apr. 24; 309(1): 11-21). The SCD activity increases in human and animals with fatty liver, but deletion of SCD1 was found to improve both the high-fat diet induced fatty liver and hereditary fatty liver (Proc Natl Acad Sci USA. 2002 Aug. 20; 99(17): 11482-6; J Biol Chem. 2000 Sep. 29; 275(39): 30132-8). It has been confirmed that SCD1 lack mouse shows resistance to diet-induced obesity, promoted energy consumption, decrease in visceral fat, and enhanced insulin signal (Proc Natl Acad Sci USA. 2002 Aug. 20; 99(17):11482-6; J Lipid Res. 2004 September; 45(9): 1674-82; Proc Natl Acad Sci USA. 2003 Sep. 16; 100(19): 11110-5). SCD1/leptin double knockout mouse is significantly nonobese as compared to control leptin deficient mouse, and shows a remarkable increase in the energy consumption amount and a significant decrease in the liver triglyceride storage and VLDL production. Therefore, suppression of SCD1 expression is considered to be an important constituent factor of a metabolic action of leptin (Science. 2002 Jul. 12; 297(5579): 240-3). Additionally, SCD1 is involved in the differentiation of adipocytes, and suggested to be also involved in food ingestion and lipolysis. Since inhibition of acetyl-CoA carboxylase 2, glycerol-3-phosphate acyltransferase, fatty acid synthase and the like involved in fatty acid synthesis cascade like SCD1 affords improvement of abnormal lipid metabolism and resistance to obesity (Science. 2001 Mar. 30; 291(5513): 2558-9; Science. 2000 Jun. 30; 288(5475): 2299-300; Proc Natl Acad Sci USA. 2002 Jul. 9; 99(14): 9498-502; Nat. Genet. 2000 May; 25(1): 6-7), control of cascade involving SCD1 is considered to be suitable as a target of disease treatment. Metabolic syndrome drawing attention in these days refers to a syndrome where a single individual shows plural symptoms of abnormal lipid metabolism, high blood pressure, abnormal sugar metabolism and the like, resulting from common onset basis such as visceral fat accumulation, insulin resistance and the like. Thus, it is a pathology with a high onset risk of cardiovascular disease and type 2 diabetes (JAMA. 2001; 285: 2486-97; Circulation 2004; 109: 433-8; Diabet. Med. 1998; 15: 539-53; The Journal of the Japanese Society of Internal Medicine 2005; 94: 188-203). According to the current guidelines, the basis of the treatment of metabolic syndrome is considered to be the improvement of lifestyle. Since prevention of the onset of cardiovascular events by the administration of statin pharmaceutical agents and fibrate pharmaceutical agents has been reported (Am J Transplant. 2005 December; 5(12): 2929-36; Lancet. 2005 Nov. 26; 366(9500): 1849-61), a novel pharmaceutical agent having an SCD inhibitory action targeting plural risk factors of metabolic syndrome is considered to be necessary also from the aspects of treatment efficiency and medical economy.

As a therapeutic drug for SCD-mediated diseases, patent documents 1-11 disclose compounds represented by the following formulas and compounds having similar structures.

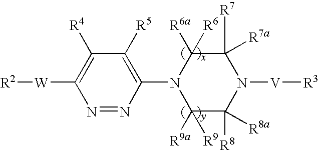

wherein each symbol is as defined in the above-mentioned documents.

However, such documents do not disclose a compound to be used as the SCD inhibitor of the present invention.

In addition, patent document 12 discloses a method, based on the administration of an SCD1 inhibitor, of treating the side effect of body weight gain associated with a drug therapy.

However, the document does not disclose a compound to be used as the SCD inhibitor of the present invention.

Moreover, patent documents 13-15 also disclose morpholine carboxamide derivatives, pyrrolidine carboxamide derivatives and piperidine carboxamide derivatives, and patent documents 16-17 disclose furancarboxamide derivatives and 1-methylpyrazole derivatives. However, these documents do not disclose that the above compounds are SCD inhibitors.

In addition, 4-[(3-bromophenoxy)methyl]-N-[3,5-dimethyl-1-(phenoxymethyl)-1H-pyrazol-4-yl]benzamide and analogous compounds thereof are registered in the CAS registry database. However, use of these compounds is not reported, and particularly, the SCD inhibitory activity thereof is not reported.

patent document 1: WO2006/086447
patent document 2: WO2006/034446
patent document 3: WO2006/034441
patent document 4: WO2006/034338
patent document 5: WO2006/034312
patent document 6: WO2006/034279
patent document 7: WO2005/011657
patent document 8: WO2005/011656
patent document 9: WO2005/011655
patent document 10: WO2005/011654
patent document 11: WO2005/011653
patent document 12: WO2006/086445
patent document 13: WO2006/014580
patent document 14: WO2001/007409
patent document 15: WO2001/000207
patent document 16: WO2005/002552
patent document 17: U.S. Pat. No. 6,414,008

### DISCLOSURE OF THE INVENTION

### Problems to be Solved by the Invention

SCD is an important control factor of lipid homeostasis and body weight control, and is considered to be a promising target of a therapeutic drug for hyperlipidemia, diabetes, obesity, abnormal lipid metabolism, fatty liver, metabolic syndrome, arteriosclerosis associated disease and cardiovascular disease. Thus, the development of a specific SCD inhibitor is desired.

### Means of Solving the Problems

The present inventors have conducted intensive studies in view of the above-mentioned problems and found that the following compound represented by the formula [I] has a superior SCD inhibitory action and shows a blood triglyceride lowering action and the like, which resulted in the completion of the present invention. Accordingly, the present invention provides the following.

[1] An SCD inhibitor comprising a compound represented by the formula [I]

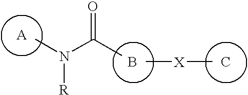

wherein
ring A is an optionally substituted aromatic ring,
ring B is an optionally substituted ring,
ring C is an optionally substituted aromatic ring,
R is a hydrogen atom, an optionally substituted hydrocarbon group or an optionally substituted heterocyclic group, and
X is a spacer having 1 to 5 atoms in the main chain, or a salt thereof, or a prodrug thereof.
[2] The agent of the above-mentioned [1], wherein the ring A is an optionally substituted aromatic cyclic hydrocarbon or an optionally substituted 5- or 6-membered monocyclic aromatic heterocycle.
[3] The agent of the above-mentioned [1], wherein the ring B is an optionally substituted aromatic cyclic hydrocarbon or an optionally substituted 5- or 6-membered nitrogen-containing heterocycle.
[4] The agent of the above-mentioned [1], wherein the ring C is an optionally substituted 6-membered aromatic ring.
[5] The agent of the above-mentioned [1], wherein X is the formula —(CH₂)ₘ—Y—(CH₂)ₙ—
wherein m and n are each an integer of 0 to 4 (total of m and n does not exceed 4), and Y is a bond (when Y is a bond, m is not 0), —O—, —S—, —S(O)—, —S(O)₂— or —N(R¹)— (wherein R¹ is a hydrogen atom, an optionally substituted hydrocarbon group or an optionally substituted heterocyclic group).
[6] The agent of the above-mentioned [1], wherein X is —CH₂—O—.
[7] The agent of the above-mentioned [1], wherein R is a hydrogen atom.
[8] The agent of the above-mentioned [1], which is an agent for the prophylaxis or treatment of hyperlipidemia.
[9] The agent of the above-mentioned [8], which further comprises a drug having a blood lipid improving effect.
[10] The agent of the above-mentioned [1], which is an agent for the prophylaxis or treatment of diabetes or obesity.
[11] A method for the prophylaxis or treatment of hyperlipidemia or obesity in a mammal, which comprises administering the agent of the above-mentioned [1] to the mammal.
[12] A method for the prophylaxis or treatment of diabetes or obesity in a mammal, which comprises administering the agent of the above-mentioned [1] to the mammal.
[13] Use of the agent of the above-mentioned [1] for the production of an agent for the prophylaxis or treatment of hyperlipidemia.
[14] Use of the agent of the above-mentioned [1] for the production of an agent for the prophylaxis or treatment of diabetes or obesity.
[15] A compound represented by the formula [II]

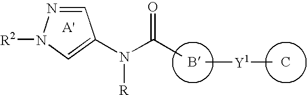

wherein
ring A′ is a further optionally substituted pyrazole ring,
ring B′ is an optionally substituted ring,
R² is an optionally substituted hydrocarbon group or an optionally substituted heterocyclic group,
ring C is an optionally substituted aromatic ring,
R is a hydrogen atom, an optionally substituted hydrocarbon group or an optionally substituted heterocyclic group, and Y¹ is —C(R³)(R⁴)—X¹—
(R³ and R⁴ are each independently a hydrogen atom, an optionally substituted hydrocarbon group, an optionally substituted heterocyclic group, an optionally substituted hydroxy group, an optionally substituted amino group, an optionally substituted mercapto group, a cyano group, a nitro group, an optionally substituted acyl group or a halogen atom, and X¹ is a spacer having 1 to 4 atoms in the main chain, provided that ring B′ is not a furan ring, R² is not a methyl group, and one of ring B′ and ring C is a heterocycle,
or a salt thereof.
[16] The compound of the above-mentioned [15], wherein ring A′ is a pyrazole ring.
[17] The compound of the above-mentioned [15], wherein ring B′ is benzene, piperidine, morpholine, pyrrolidine or pyridine.
[18] The compound of the above-mentioned [15], wherein R² is an optionally substituted C₁₋₆ alkyl group, an optionally substituted C₇₋₁₂ aralkyl group, optionally substituted C₆₋₁₀ aryl group or an optionally substituted 5- or 6-membered nitrogen-containing heterocyclic group.
[19] The compound of the above-mentioned [15], wherein ring C is an optionally substituted 6-membered aromatic ring.
[20] The compound of the above-mentioned [15], wherein R is a hydrogen atom.
[21] The compound of the above-mentioned [15], wherein Y¹ is —CH₂O—, —CH₂CH₂— or —CH₂CH₂O—.
[22] 3-[(2-Chlorophenoxy)methyl]-N-[1-(4-fluorobenzyl)-1H-pyrazol-4-yl]pyrrolidine-1-carboxamide;
4-[(2-chlorophenoxy)methyl]-N-[1-(2-hydroxyethyl)-1H-pyrazol-4-yl]pyridine-2-carboxamide;
4-[(2-chloro-5-fluorophenoxy)methyl]-N-[1-(4-fluorobenzyl)-1H-pyrazol-4-yl]pyridine-2-carboxamide;
N-[1-(4-fluorobenzyl)-1H-pyrazol-4-yl]-4-{[2-fluoro-5-(trifluoromethyl)phenoxy]methyl}pyridine-2-carboxamide;
4-[(2,5-dichlorophenoxy)methyl]-N-[1-(pyridin-2-ylmethyl)-1H-pyrazol-4-yl]pyridine-2-carboxamide;
   or a salt thereof.
   [23] A prodrug of the compound of the above-mentioned [15].
   [24] A pharmaceutical agent comprising the compound of the above-mentioned [15] or a prodrug thereof.
   [25] The pharmaceutical agent of the above-mentioned [24], which is an SCD inhibitor.
   [26] The pharmaceutical agent of the above-mentioned [24], which is an agent for the prophylaxis or treatment of hyperlipidemia.
   [27] The pharmaceutical agent of the above-mentioned [26], further comprising a drug having a blood lipid improving effect.
   [28] The pharmaceutical agent of the above-mentioned [24], which is an agent for the prophylaxis or treatment of diabetes or obesity.
   [29] A method for the prophylaxis or treatment of hyperlipidemia in a mammal, which comprises administering the compound of the above-mentioned [15] or a prodrug thereof to the mammal.
   [30] A method for the prophylaxis or treatment of diabetes or obesity in a mammal, which comprises administering the compound of the above-mentioned [15] or a prodrug thereof to the mammal.
   [31] Use of the compound of the above-mentioned [15] or a prodrug thereof for the production of an agent for the prophylaxis or treatment of hyperlipidemia.
   [32] Use of the compound of the above-mentioned [15] or a prodrug thereof for the production of an agent for the prophylaxis or treatment of diabetes or obesity.
   [33] A compound represented by the formula [III]

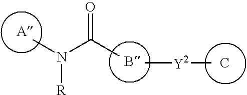

wherein
ring A″ is an optionally substituted aromatic heterocycle,

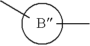

is a ring selected from

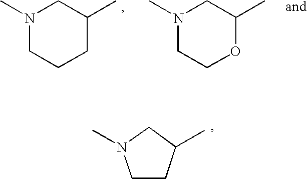

each of which is optionally substituted,
ring C is an optionally substituted aromatic ring,
R is a hydrogen atom, an optionally substituted hydrocarbon group or an optionally substituted heterocyclic group,

### Y² is —C(R³) (R⁴)—X²—

wherein R³ and R⁴ are each independently a hydrogen atom, an optionally substituted hydrocarbon group, an optionally substituted heterocyclic group, an optionally substituted hydroxy group, an optionally substituted amino group, an optionally substituted mercapto group, a cyano group, a nitro group, an optionally substituted acyl group or a halogen atom, and
X² is a spacer having 1 to 4 atoms in the main chain, ring A″ is not pyrazol-4-yl having a substituent at the 1-position,

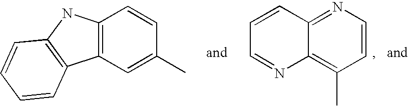

### X² is not —NH—,

or a salt thereof.
[34] The compound of the above-mentioned [33], wherein ring A″ is an optionally substituted 5- or 6-membered nitrogen-containing aromatic heterocycle.
[35] The compound of the above-mentioned [33], wherein ring B″ is unsubstituted.
[36] The compound of the above-mentioned [33], wherein ring C is an optionally substituted 6-membered aromatic ring.
[37] The compound of the above-mentioned [33], wherein R is a hydrogen atom.
[38] The compound of the above-mentioned [33], wherein Y² is —CH₂O—, —CH₂CH₂— or —CH₂CH₂O—.
[39] N-(4,6-Dimethylpyridin-2-yl)-2-[(2-fluorophenoxy)methyl]morpholine-4-carboxamide;
N-(4,6-dimethylpyridin-2-yl)-2-{[2-(trifluoromethyl)phenoxy]methyl}morpholine-4-carboxamide;
2-[(2-chloro-5-fluorophenoxy)methyl]-N-(4,6-dimethylpyridin-2-yl)morpholine-4-carboxamide;
2-[(2-chloro-5-fluorophenoxy)methyl]-N-(6-ethylpyridin-2-yl) morpholine-4-carboxamide;
2-[(2-chloro-5-fluorophenoxy)methyl]-N-[5-(hydroxymethyl)pyridin-2-yl]morpholine-4-carboxamide;
   or a salt thereof.
   [40] A prodrug of the compound of the above-mentioned [33].
   [41] A pharmaceutical agent comprising the compound of the above-mentioned [33] or a prodrug thereof.
   [42] The pharmaceutical agent of the above-mentioned [41], which is an SCD inhibitor.
   [43] The pharmaceutical agent of the above-mentioned [41], which is an agent for the prophylaxis or treatment of hyperlipidemia.
   [44] The pharmaceutical agent of the above-mentioned [43], further comprising a drug having a blood lipid improving effect.
   [45] The pharmaceutical agent of the above-mentioned [41], which is an agent for the prophylaxis or treatment of diabetes or obesity.
   [46] A method for the prophylaxis or treatment of hyperlipidemia in a mammal, which comprises administering the compound of the above-mentioned [33] or a prodrug thereof to the mammal.
   [47] A method for the prophylaxis or treatment of diabetes or obesity in a mammal, which comprises administering the compound of the above-mentioned [33] or a prodrug thereof to the mammal.
   [48] Use of the compound of the above-mentioned [33] or a prodrug thereof for the production of an agent for the prophylaxis or treatment of hyperlipidemia.
   [49] Use of the compound of the above-mentioned [33] or a prodrug thereof for the production of an agent for the prophylaxis or treatment of diabetes or obesity.

In the present specification, a compound represented by the formula [I], a compound represented by the formula [II] or a compound represented by the formula [III] are sometimes to be abbreviated as compound (I), compound (II) and compound (III), respectively. Other compounds are also abbreviated similarly.

### Effect of the Invention

Compound (I), compound (II) and compound (III) show an SCD inhibitory action (particularly SCD-1 inhibitory action), and the compounds are considered to show a fatty acid desaturation inhibitory action, an insulin signal enhancing action, suppression of body weight gain and a visceral fat-decreasing action based on a promoted energy consumption, plasma and liver triglyceride lowering action, cholesterol ester and lipoprotein synthesis inhibitory action, and cholesterol efflux improving effect via ATP-binding cassette transporter A1 (ABCA1), and are highly useful as a prophylactic or therapeutic agent for hyperlipidemia (including hypercholesterolemia, high LDL-cholesterolemia, low HDL-cholesterolemia and hypertriglyceride(TG)mia and the like, particularly, hypertriglyceridemia), diabetes (including type 1 diabetes, type 2 diabetes, gestational diabetes, obese diabetes and the like, particularly, type 2 diabetes), obesity, abnormal lipid metabolism, fatty liver, metabolic syndrome, arteriosclerosis associated disease and fatal myocardial infarction, sudden cardiac death, nonfatal myocardial infarction, angina pectoris decubitus or effort angina pectoris, instabilization of angina pectoris, cardio- and cerebrovascular disorders (cardiovascular diseases including cerebral thrombus, cerebral embolism, cerebral hemorrhage, subarachnoid hemorrhage, TIA (transient cerebral ischemic attack; Transient ischemic attack)) and the like.

### DETAILED DESCRIPTION OF THE INVENTION

The present invention is explained in detail in the following.

Examples of the “halogen atom” to be used in the present specification include fluorine, chlorine, bromine and iodine.

Examples of the “aromatic ring group” of the “optionally substituted aromatic ring group” to be used in the present specification include aromatic cyclic hydrocarbon, aromatic heterocycle (e.g., monocyclic aromatic heterocycle, fused aromatic heterocycle) and the like.

Examples of the “aromatic cyclic hydrocarbon” include C₆₋₁₄ aromatic cyclic hydrocarbon (preferably C₆₋₁₂ aromatic cyclic hydrocarbon) such as benzene, naphthalene, anthracene, phenanthrene, acenaphthylene etc., and the like.

Examples of the “aromatic heterocycle” include 5- or 6-membered monocyclic aromatic heterocycle such as furan, thiophene, pyrrole, oxazole, isoxazole, thiazole, isothiazole, imidazole, pyrazole, 1,2,3-oxadiazole, 1,2,4-oxadiazole, 1,3,4-oxadiazole, furazan, 1,2,3-thiadiazole, 1,2,4-thiadiazole, 1,3,4-thiadiazole, 1,2,3-triazole, 1,2,4-triazole, tetrazole, pyridine, pyridazine, pyrimidine, pyrazine, triazine etc.;

8- to 16-membered (preferably, 8- to 12-membered) fused aromatic heterocycle such as benzofuran, isobenzofuran, pyrazolothiophene, benzo[b]thiophene, benzo[c]thiophene, indole, isoindole, 1H-indazole, benzoimidazole, benzoxazole, 1,2-benzoisoxazole, benzothiazole, 1,2-benzoisothiazole, 1H-benzotriazole, quinoline, isoquinoline, cinnoline, quinazoline, quinoxaline, phthalazine, naphthyridine, purine, pteridinee, carbazole, α-carboline, β-carboline, γ-carboline, acridine, phenoxazine, phenothiazine, phenazine, phenoxathiine, thianthrene, phenanthridine, phenanthrolin, indolizine, pyrrolopyridine, pyrrolo[1,2-b]pyridazine, pyrazolo[1,5-a]pyridine, imidazo[1,2-a]pyridine, imidazo[1,5-a]pyridine, imidazo[1,2-b]pyridazine, imidazo[1,2-a]pyrimidine, 1,2,4-triazolo[4,3-a]pyridine, 1,2,4-triazolo[4,3-b]pyridazine, thienopyrazine etc. (preferably, heterocycle wherein 1 or 2 (preferably, 1) of the aforementioned 5- or 6-membered monocyclic aromatic heterocycle and 1 or 2 (preferably, 1) benzene ring are fused, and heterocycle wherein 2 or 3 (preferably 2), the same or different heterocycles of the aforementioned 5- or 6-membered monocyclic aromatic heterocycle are fused) and the like.

In the formula [I], the “aromatic ring” of the “optionally substituted aromatic ring” for ring A is preferably an aromatic cyclic hydrocarbon or aromatic heterocycle.

The aromatic cyclic hydrocarbon is preferably C₆₋₁₄ arene, more preferably C₆₋₁₀ arene, and benzene is particularly preferable.

As the aromatic heterocycle, a 5- or 6-membered monocyclic aromatic heterocycle (e.g., a 5-membered ring containing, besides carbon atom, 1-4 hetero atoms selected from a nitrogen atom, an oxygen atom and a sulfur atom such as thiophene, furan, oxazole, isoxazole, thiazole, isothiazole, thiadiazole, imidazole, pyrazole, triazole, tetrazole and the like, a 6-membered ring containing, besides carbon atom, 1-4 hetero atoms selected from a nitrogen atom, an oxygen atom and a sulfur atom such as pyridine, pyrimidine, triazine, pyridazine, pyrazine and the like) is preferable. Specifically, pyrazole, pyridine, pyrimidine, pyrazine, triazole, thiazole, isothiazole, thiadiazole, pyridazine, thiophene and isoxazole are preferable.

As the “aromatic ring” of the “optionally substituted aromatic ring” for ring A, a ring wherein the above-mentioned aromatic cyclic hydrocarbon (preferably, C₆₋₁₀ arene group) and the above-mentioned 5- or 6-membered aromatic heterocycle are fused (e.g., benzothiazole) is also preferable.

Ring A can be bonded to a nitrogen atom of the NR group in the formula [I] at a bindable position.

In the formulas [I], [II] and [III], as the “aromatic ring” of the “optionally substituted aromatic ring” for ring C, a 5- or 6-membered aromatic ring is preferable. Specifically, benzene, pyrazole, pyridine, pyrimidine, imidazole and the like can be mentioned. Moreover, a ring wherein the above-mentioned 5- or 6-membered aromatic ring and heterocycle are fused is also preferable, and benzoimidazole, indazole, imidazopyridine, benzoxazole, benzoxazine can be specifically mentioned.

The “aromatic ring” of the “optionally substituted aromatic ring” for ring C is particularly preferably a 6-membered aromatic ring, specifically, benzene, pyridine or pyrimidine.

The ring C in the formulas [I], [II] and [III] can be bonded to X group, Y¹ group and Y² group, respectively, at a bindable position.

Examples of the “substituent” of the “optionally substituted aromatic ring” include

(1) a halogen atom (e.g., fluorine, chlorine, bromine, iodine),
(2) a C₁₋₆ alkyl group (e.g., methyl, ethyl, propyl, isopropyl, butyl, isobutyl, sec-butyl, tert-butyl, pentyl, hexyl),
(3) a C₃₋₆ cycloalkyl group (e.g., cyclopropyl, cyclobutyl, cyclopentyl, cyclohexyl),
(4) a C₂₋₆ alkynyl group (e.g., ethynyl, 1-propynyl, propargyl),
(5) a C₂₋₆ alkenyl group (e.g., vinyl, allyl, isopropenyl, butenyl, isobutenyl),
(6) a C₇₋₁₂ aralkyl group (e.g., benzyl, α-methylbenzyl, phenethyl),
(7) a C₆₋₁₀ aryl group (e.g., phenyl, naphthyl, preferably phenyl group),
(8) a C₁₋₆ alkoxy group (e.g., methoxy, ethoxy, propoxy, isopropoxy, butoxy, isobutoxy, sec-butoxy, tert-butoxy),
(9) a C₆₋₁₀ aryloxy group (e.g., phenoxy),
(10) a formyl group or a C₁₋₆ alkyl-carbonyl group (e.g., acetyl, propionyl, butyryl, isobutyryl),
(11) a C₆₋₁₀ aryl-carbonyl group (e.g., benzoyl, naphthoyl),
(12) a formyloxy group or a C₁₋₆ alkyl-carbonyloxy group (e.g., acetyloxy, propionyloxy, butyryloxy, isobutyryloxy),
(13) a C₆₋₁₀ aryl-carbonyloxy group (e.g., benzoyloxy, naphthoyloxy),
(14) a carboxyl group,
(15) a C₁₋₆ alkoxy-carbonyl group (e.g., methoxycarbonyl, ethoxycarbonyl, propoxycarbonyl, isopropoxycarbonyl, butoxycarbonyl, isobutoxycarbonyl, tert-butoxycarbonyl),
(16) a C₇₋₁₂ aralkyloxy-carbonyl group (e.g., benzyloxycarbonyl),
(17) a carbamoyl group,
(18) a mono-, di- or tri-halogeno-C₁₋₆ alkyl group (e.g., chloromethyl, dichloromethyl, trifluoromethyl, 2,2,2-trifluoroethyl),
(19) an oxo group,
(20) an amidino group,
(21) an imino group,
(22) an amino group,
(23) a mono-C₁₋₆ alkylamino group (e.g., methylamino, ethylamino, propylamino, isopropylamino, butylamino),
(24) a di-C₁₋₆ alkylamino group (e.g., dimethylamino, diethylamino, dipropylamino, diisopropylamino, dibutylamino, N-ethyl-N-methylamino),
(25) a 3- to 8-membered nitrogen-containing heterocyclic group optionally having substituents and optionally containing, besides carbon atom and one nitrogen atom, 1-3 hetero atoms selected from a nitrogen atom, an oxygen atom and a sulfur atom
(e.g., a 3- to 8-membered nitrogen-containing heterocyclic group optionally containing, besides carbon atom and one nitrogen atom, 1-3 hetero atoms selected from a nitrogen atom, an oxygen atom and a sulfur atom and optionally having 1-5 substituents selected from a halogen atom, a nitro group, a cyano group, a hydroxy group, an optionally halogenated C₁₋₆ alkyl group (e.g., methyl, ethyl, propyl, isopropyl, butyl, isobutyl, sec-butyl, tert-butyl, pentyl, hexyl), an optionally halogenated C₁₋₆ alkoxy group (e.g., methoxy, ethoxy, propoxy, isopropoxy, butoxy, isobutoxy, sec-butoxy, tert-butoxy), an amino group, a mono-C₁₋₆ alkylamino group (e.g., methylamino, ethylamino, propylamino, isopropylamino, butylamino), a di-C₁₋₆ alkylamino group (e.g., dimethylamino, diethylamino, dipropylamino, diisopropylamino, dibutylamino, N-ethyl-N-methylamino), a carboxyl group, a C₁₋₆ alkyl-carbonyl group (e.g., acetyl, propionyl, butyryl, isobutyryl), a C₁₋₆ alkoxy-carbonyl group (e.g., methoxycarbonyl, ethoxycarbonyl, propoxycarbonyl, isopropoxycarbonyl, butoxycarbonyl, isobutoxycarbonyl, tert-butoxycarbonyl), a carbamoyl group, a mono-C₁₋₆ alkyl-carbamoyl group (e.g., methylcarbamoyl, ethylcarbamoyl, propylcarbamoyl, isopropylcarbamoyl, butylcarbamoyl, isobutylcarbamoyl, sec-butyl, pentylcarbamoyl, hexylcarbamoyl), a di-C₁₋₆ alkyl-carbamoyl group (e.g., dimethylcarbamoyl, diethylcarbamoyl, dipropylcarbamoyl), a C₆₋₁₀ aryl-carbamoyl group (e.g., phenylcarbamoyl, naphthylcarbamoyl), a C₆₋₁₀ aryl group (e.g., phenyl, naphthyl), a C₆₋₁₀ aryloxy group (e.g., phenoxy), an optionally halogenated C₁₋₆ alkyl-carbonylamino group (e.g., acetylamino, propionylamino, butyrylamino, isobutyrylamino), an oxo group and the like; for example, aziridinyl, azetidinyl, pyrrolidinyl, pyridyl, pyrrolinyl, pyrrolyl, imidazolyl, pyrazolyl, imidazolidinyl, piperidyl, oxadiazolyl, isoxazolyl, morpholinyl, dihydropyridyl, tetrahydropyridyl, piperazinyl, N-methylpiperazinyl, N-ethylpiperazinyl),
(26) a C₁₋₃ alkylenedioxy group (e.g., methylenedioxy, ethylenedioxy),
(27) a hydroxy group,
(28) a nitro group,
(29) a cyano group,
(30) a mercapto group,
(31) a sulfo group,
(32) a sulfino group,
(33) a phosphono group,
(34) a sulfamoyl group,
(35) a mono-C₁₋₆ alkylsulfamoyl group (e.g., N-methylsulfamoyl, N-ethylsulfamoyl, N-propylsulfamoyl, N-isopropylsulfamoyl, N-butylsulfamoyl),
(36) a di-C₁₋₆ alkylsulfamoyl group (e.g., N,N-dimethylsulfamoyl, N,N-diethylsulfamoyl, N,N-dipropylsulfamoyl, N,N-dibutylsulfamoyl),
(37) a C₁₋₆ alkylthio group (e.g., methylthio, ethylthio, propylthio, isopropylthio, butylthio, sec-butylthio, tert-butylthio),
(38) a C₆₋₁₀ arylthio group (e.g., phenylthio, naphthylthio),
(39) a C₁₋₆ alkylsulfinyl group (e.g., methylsulfinyl, ethylsulfinyl, propylsulfinyl, butylsulfinyl),
(40) a C₆₋₁₀ arylsulfinyl group (e.g., phenylsulfinyl, naphthylsulfinyl),
(41) a C₁₋₆ alkylsulfonyl group (e.g., methylsulfonyl, ethylsulfonyl, propylsulfonyl, butylsulfonyl),
(42) a C₆₋₁₀ arylsulfonyl group (e.g., phenylsulfonyl, naphthylsulfonyl)
(in the present specification, the above-mentioned substituents are collectively referred to as substituent group (a)) and the like.

The “aromatic ring” of the “optionally substituted aromatic ring” optionally has 1-5, preferably 1-3 of the aforementioned substituents at substitutable position(s) of the aromatic ring. When the number of the substituents is two or more, the substituents may be the same or different. Moreover, these substituents are optionally substituted by 1 to 3 substituents from substituent group (a) at substitutable position(s).

As the substituent of the “aromatic ring” of the “optionally substituted aromatic ring” for ring A,

(1) a C₁₋₆ alkyl group (e.g., methyl, ethyl, propyl, isopropyl, butyl, isobutyl, sec-butyl, tert-butyl, pentyl, hexyl) optionally substituted by 1 to 3 substituents selected from
(a) a 3- to 8-membered nitrogen-containing heterocyclic group (preferably, pyrazolyl, pyrrolyl, pyridyl etc.),
(b) a halogen atom,
(c) a hydroxy group, and
(d) a C₃₋₆ cycloalkyl group (e.g., cyclopropyl, cyclobutyl, cyclopentyl, cyclohexyl, preferably cyclopropyl group),
   (2) a hydroxy group optionally substituted by a C₇₋₁₂ aralkyl group (e.g., benzyl, α-methylbenzyl, phenethyl),
   (3) a C₇₋₁₂ aralkyl group (e.g., benzyl, α-methylbenzyl, phenethyl) optionally substituted by 1 to 3 halogen atoms,
   (4) a C₆₋₁₀ aryl group (e.g., phenyl, naphthyl, preferably phenyl group) optionally substituted by 1 to 3 halogen atoms,
   (5) a halogen atom,
   (6) a carbamoyl group,
   (7) a 3- to 8-membered nitrogen-containing heterocyclic group (e.g., pyridyl),
   (8) a C₆₋₁₀ aryloxy group (e.g., phenoxy) optionally substituted by 1 to 3 halogen atoms,
   (9) a C₁₋₆ alkoxy group (e.g., methoxy, ethoxy, propoxy, isopropoxy, butoxy, isobutoxy, sec-butoxy, tert-butoxy)
   and the like are preferable.

The “optionally substituted aromatic ring” for ring A is preferably

optionally substituted aromatic cyclic hydrocarbon (preferably, benzene) or optionally substituted 5- or 6-membered monocyclic aromatic heterocycle (preferably, pyrazole, pyridine, pyrimidine, pyrazine, triazole, thiadiazole, thiazole, isothiazole, pyridazine, thiophene, isoxazole), more preferably
aromatic cyclic hydrocarbon (preferably, benzene) and 5- or 6-membered monocyclic aromatic heterocycle (preferably, pyrazole, pyridine, pyrimidine, pyrazine, triazole, thiazole, isothiazole, pyridazine, thiophene, isoxazole), each optionally substituted by 1 to 3 substituents selected from
(1) a C₁₋₆ alkyl group (e.g., methyl, ethyl, propyl, isopropyl, butyl, isobutyl, sec-butyl, tert-butyl, pentyl, hexyl) optionally substituted by 1 to 3 substituents selected from
(a) a 3- to 8-membered nitrogen-containing heterocyclic group (preferably, pyrazolyl, pyrrolyl, pyridyl etc.),
(b) a halogen atom,
(c) a hydroxy group, and
(d) a C₃₋₆ cycloalkyl group (e.g., cyclopropyl, cyclobutyl, cyclopentyl, cyclohexyl, preferably cyclopropyl group),
   (2) a hydroxy group optionally substituted by a C₇₋₁₂ aralkyl group (e.g., benzyl, α-methylbenzyl, phenethyl),
   (3) a C₇₋₁₂ aralkyl group (e.g., benzyl, α-methylbenzyl, phenethyl) optionally substituted by 1 to 3 halogen atoms,
   (4) a C₆₋₁₀ aryl group (e.g., phenyl, naphthyl, preferably phenyl group) optionally substituted by 1 to 3 halogen atoms,
   (5) a halogen atom,
   (6) a carbamoyl group,
   (7) a 3- to 8-membered nitrogen-containing heterocyclic group (e.g., pyridyl), and
   (8) a C₆₋₁₀ aryloxy group (e.g., phenoxy) optionally substituted by 1 to 3 halogen atoms.

As the substituent of the “aromatic ring” of the “optionally substituted aromatic ring” for ring C,

(1) a C₁₋₆ alkyl group (e.g., methyl, ethyl, propyl, isopropyl, butyl, isobutyl, sec-butyl, tert-butyl, pentyl, hexyl) optionally substituted by 1 to 3 halogen atoms,
(2) a 3- to 8-membered nitrogen-containing heterocyclic group (e.g., isoxazolyl, pyrazolyl, oxadiazolyl) optionally substituted by 1 to 3 C₁₋₆ alkyl groups (e.g., methyl, ethyl, propyl, isopropyl, butyl, isobutyl, sec-butyl, tert-butyl, pentyl, hexyl),
(3) a C₁₋₆ alkoxy group (e.g., methoxy, ethoxy, propoxy, isopropoxy, butoxy, isobutoxy, sec-butoxy, tert-butoxy) optionally substituted by 1 to 3 halogen atoms,
(4) a C₁₋₆ alkoxy-carbonyl group (e.g., methoxycarbonyl, ethoxycarbonyl, propoxycarbonyl, isopropoxycarbonyl, butoxycarbonyl, isobutoxycarbonyl, tert-butoxycarbonyl),
(5) an oxo group,
(6) a halogen atom,
(7) a cyano group,
and the like are preferable.

As the “optionally substituted aromatic ring” for ring C, an optionally substituted 6-membered aromatic ring (preferably, benzene, pyridine, pyrimidine) is preferable and a 6-membered aromatic ring (preferably, benzene, pyridine, pyrimidine) optionally substituted by 1 to 3 substituents selected from

(1) a C₁₋₆ alkyl group (e.g., methyl, ethyl, propyl, isopropyl, butyl, isobutyl, sec-butyl, tert-butyl, pentyl, hexyl) optionally substituted by 1 to 3 halogen atoms,
(2) a 3- to 8-membered nitrogen-containing heterocyclic group (e.g., isoxazolyl, pyrazolyl, oxadiazolyl) optionally substituted by 1 to 3 C₁₋₆ alkyl groups (e.g., methyl, ethyl, propyl, isopropyl, butyl, isobutyl, sec-butyl, tert-butyl, pentyl, hexyl),
(3) a C₁₋₆ alkoxy group (e.g., methoxy, ethoxy, propoxy, isopropoxy, butoxy, isobutoxy, sec-butoxy, tert-butoxy) optionally substituted by 1 to 3 halogen atoms,
(4) a C₁₋₆ alkoxy-carbonyl group (e.g., methoxycarbonyl, ethoxycarbonyl, propoxycarbonyl, isopropoxycarbonyl, butoxycarbonyl, isobutoxycarbonyl, tert-butoxycarbonyl),
(5) an oxo group,
(6) a halogen atom, and
(7) a cyano group are more preferable.

Examples of the “ring” of the “optionally substituted ring” for ring B in the formula [I] to be used in the present specification include aromatic cyclic hydrocarbon, aromatic heterocycle, nonaromatic cyclic hydrocarbon, non-aromatic heterocycle, fused rings thereof and the like. Examples of the “aromatic cyclic hydrocarbon” and “aromatic heterocycle” include those exemplified as the “optionally substituted aromatic ring” for the above-mentioned ring A or ring C.

Examples of the “nonaromatic cyclic hydrocarbon” include cycloalkane, cycloalkene, cycloalkadiene and the like, each optionally fused with benzene ring, specifically, C₃₋₁₀ cycloalkane (e.g., cyclopropane, cyclobutane, cyclopentane, cyclohexane, cycloheptane, cyclooctane, cyclononane, cyclodecane), C₃₋₁₀ cycloalkene (e.g., cyclopropene, cyclobutene, cyclopentene, cyclohexene, cycloheptene, cyclooctene, cyclononene, cyclodecene), C₄₋₁₀ cycloalkadiene (e.g., cyclobutadiene, cyclopentadiene, cyclohexadiene, cycloheptadiene, cyclooctadiene, cyclononadiene, cyclodecadiene), a fused ring wherein these rings and a benzene ring are fused (e.g., inden, tetrahydronaphthalene (e.g., 1,2,3,4-tetrahydronaphthalene), fluorene etc.) and the like.

Examples of the “non-aromatic heterocycle” include 3 to 8-membered (preferably 5- or 6-membered) saturated or unsaturated (preferably saturated) non-aromatic heterocycle and the like, specifically, oxirane, azetidine, oxetane, thietane, pyrrolidine, tetrahydrofuran, thioran, piperidine, tetrahydropyran, thiane, morpholine, thiomorpholine, piperazine, azepane, oxepane, thiepane, oxazepane, thiazepane, azokane, oxokane, thiokane, oxazokane, thiazokane, dioxine and the like.

The “ring” of the “optionally substituted ring” for ring B is preferably aromatic cyclic hydrocarbon, aromatic heterocycle or non-aromatic heterocycle.

The aromatic cyclic hydrocarbon is preferably C₆₋₁₄ arene, C₆₋₁₀ arene is more preferable, and benzene is particularly preferable.

The aromatic heterocycle is preferably pyridine, pyrazolothiophene, furan, pyrazole, thiophene, benzofuran, indole and the like.

The non-aromatic heterocycle is preferably piperazine, piperidine, pyrrolidine, morpholine and the like.

The aromatic heterocycle and non-aromatic heterocycle is particularly preferably 5- or 6-membered heterocycle containing one or more nitrogen atoms such as pyridine, pyrazole, morpholine, piperidine, pyrrolidine and the like.

In the present specification, 5- or 6-membered heterocycle containing one or more nitrogen atoms in a molecule is referred to as 5- or 6-membered nitrogen-containing heterocycle.

As the “substituent” of the “optionally substituted ring”, those exemplified in the above-mentioned substituent group (a) can be mentioned.

The “ring” of the “optionally substituted ring” may have 1-5, preferably 1-3, of the aforementioned substituents at substitutable position(s) of the ring, and when the number of the substituents is two or more, the substituents may be the same or different. These substituents are optionally substituted by substituent group (a).

As the substituent of the “optionally substituted ring” for ring B,

(1) a C₁₋₆ alkyl group (e.g., methyl, ethyl, propyl, isopropyl, butyl, isobutyl, sec-butyl, tert-butyl, pentyl, hexyl),
(2) a C₁₋₆ alkoxy group (e.g., methoxy, ethoxy, propoxy, isopropoxy, butoxy, isobutoxy, sec-butoxy, tert-butoxy)
and the like are preferable.

The “optionally substituted ring” for ring B is preferably optionally substituted aromatic cyclic hydrocarbon (preferably, benzene) or optionally substituted 5- or 6-membered nitrogen-containing heterocycle (preferably, pyridine, pyrazole, morpholine, piperidine, pyrrolidine), and more preferably

aromatic cyclic hydrocarbon (preferably, benzene) or 5- or 6-membered nitrogen-containing heterocycle (preferably, pyridine, pyrazole, morpholine, piperidine, pyrrolidine), optionally substituted by 1 to 3 substituents selected from (1) a C₁₋₆ alkyl group (e.g., methyl, ethyl, propyl, isopropyl, butyl, isobutyl, sec-butyl, tert-butyl, pentyl, hexyl), and
(2) a C₁₋₆ alkoxy group (e.g., methoxy, ethoxy, propoxy, isopropoxy, butoxy, isobutoxy, sec-butoxy, tert-butoxy).

Examples of the “hydrocarbon group” of the “optionally substituted hydrocarbon group” for R in the formula [I], the formula [II] and the formula [III] include an aliphatic hydrocarbon group, a monocyclic saturated hydrocarbon group, an aromatic hydrocarbon group and the like, with preference given to one having 1-16 carbons. Specifically, for example, alkyl group, alkenyl group, alkynyl group, cycloalkyl group, aryl group and the like are used.

The “alkyl group” is preferably a C₁₋₆ alkyl group and the like and, for example, methyl, ethyl, propyl, isopropyl, butyl, isobutyl, sec-butyl, tert-butyl, pentyl, hexyl and the like are widely used.

The “alkenyl group” is preferably a C₂₋₆ alkenyl group and the like and, for example, vinyl, 1-propenyl, allyl, isopropenyl, butenyl, isobutenyl and the like are widely used.

The “alkynyl group” is preferably a C₂₋₆ alkynyl group and the like and, for example, ethynyl, propargyl, 1-propynyl and the like are widely used.

The “cycloalkyl group” is preferably a C₃₋₆ cycloalkyl group and the like, and, for example, cyclopropyl, cyclobutyl, cyclopentyl and cyclohexyl are widely used.

The “aryl group” is preferably a C₆₋₁₄ aryl group such as phenyl, 1-naphthyl, 2-naphthyl, biphenylyl, 2-anthryl and the like, and the like, and a C₆₋₁₀ aryl group is more preferable, and, for example, a phenyl group and the like are widely used.

The “hydrocarbon group” of the “optionally substituted hydrocarbon group” for R is preferably a C₁₋₆ alkyl group (e.g., methyl, ethyl, propyl, butyl) or a C₃₋₆ cycloalkyl group (e.g., cyclopropyl, cyclopentyl, cyclohexyl).

The “substituent” of the “optionally substituted hydrocarbon group” is, for example, those exemplified as the above-mentioned substituent group (a).

The “hydrocarbon group” of the “optionally substituted hydrocarbon group” may have 1-5, preferably 1-3, of the aforementioned substituents at substitutable position(s) of the hydrocarbon group and, when the number of the substituents is two or more, the substituents may be the same or different. Moreover, these substituents are optionally substituted by substituent group (a).

In the formula [I], the formula [II] and the formula [III], the “heterocyclic group” of the “optionally substituted heterocyclic group” for R is, for example, an “aromatic heterocyclic group” or a “nonaromatic heterocyclic group”.

Examples of the aromatic heterocyclic group include a 5- to 7-membered monocyclic aromatic heterocyclic group containing, as ring-constituting atom besides carbon atom, 1 to 4 hetero atoms selected from an oxygen atom, a sulfur atom and a nitrogen atom and a fused aromatic heterocyclic group. Examples of the fused aromatic heterocyclic group include a group wherein such 5- to 7-membered monocyclic aromatic heterocyclic group and 1 or 2 from a 5- or 6-membered ring containing 1 or 2 nitrogen atoms, a 5-membered ring containing one sulfur atom, a benzene ring and the like are fused and the like.

Preferable examples of the aromatic heterocyclic group include

monocyclic aromatic heterocyclic group such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl, 6-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 4-isothiazolyl), oxazolyl (e.g., 2-oxazolyl, 4-oxazolyl, 5-oxazolyl), isoxazolyl, oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl), triazinyl (e.g., 1,2,4-triazol-1-yl, 1,2,4-triazol-3-yl) and the like; fused aromatic heterocyclic group such as quinolyl (e.g., 2-quinolyl, 3-quinolyl, 4-quinolyl, 6-quinolyl), isoquinolyl (e.g., 3-isoquinolyl), quinazolyl (e.g., 2-quinazolyl, 4-quinazolyl), quinoxalyl (e.g., 2-quinoxalyl, 6-quinoxalyl), benzofuryl (e.g., 2-benzofuryl, 3-benzofuryl), benzothienyl (e.g., 2-benzothienyl, 3-benzothienyl), benzoxazolyl (e.g., 2-benzoxazolyl), benzoisoxazolyl (e.g., 7-benzoisoxazolyl), benzothiazolyl (e.g., 2-benzothiazolyl), benzoimidazolyl (e.g., benzoimidazol-1-yl, benzoimidazol-2-yl, benzoimidazol-5-yl), benzotriazolyl (e.g., 1H-1,2,3-benzotriazol-5-yl), indolyl (e.g., indol-1-yl, indol-2-yl, indol-3-yl, indol-5-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridinyl (e.g., 1H-imidazo[4,5-b]pyridin-2-yl, 1H-imidazo[4,5-c]pyridin-2-yl, 2H-imidazo[1,2-a]pyridin-3-yl), imidazopyrazinyl (e.g., 1H-imidazo[4,5-b]pyrazin-2-yl), pyrazolopyridinyl (e.g., 1H-pyrazolo[4,3-c]pyridin-3-yl), pyrazolothienyl (e.g., 2H-pyrazolo[3,4-b]thiophen-2-yl), pyrazolotriazinyl (e.g., pyrazolo[5,1-c][1,2,4]triazin-3-yl) and the like; and the like.

Examples of the nonaromatic heterocyclic group include a 5- to 7-membered monocyclic nonaromatic heterocyclic group containing, as ring-constituting atom besides carbon atom, 1 to 4 hetero atoms selected from an oxygen atom, a sulfur atom and a nitrogen atom and a fused nonaromatic heterocyclic group. Examples of the fused nonaromatic heterocyclic group include a group wherein such 5- to 7-membered monocyclic nonaromatic heterocyclic group and 1 or 2 from a 5- or 6-membered ring containing 1 or 2 nitrogen atoms, a 5-membered ring containing one sulfur atom, a benzene ring and the like are fused and the like.

Preferable examples of the nonaromatic heterocyclic group include

monocyclic nonaromatic heterocyclic group such as pyrrolidinyl (e.g., 1-pyrrolidinyl), piperidinyl (e.g., piperidino), morpholinyl (e.g., morpholino), thiomorpholinyl (e.g., thiomorpholino), piperazinyl (e.g., 1-piperazinyl), hexamethyleniminyl (e.g., hexamethylenimine-1-yl), oxazolidinyl (e.g., oxazolidin-3-yl), thiazolidinyl (e.g., thiazolidin-3-yl), imidazolidinyl (e.g., imidazolidin-3-yl), dioxolyl (e.g., 1,3-dioxol-4-yl), dioxolanyl (e.g., 1,3-dioxolan-4-yl), dihydroxadiazolyl (e.g., 4,5-dihydro-1,2,4-oxadiazol-3-yl), 2-thioxo-1,3-oxazolidin-5-yl, tetrahydropyranyl (e.g., 4-tetrahydropyranyl) and the like;
fused nonaromatic heterocyclic group such as dihydroisoindolyl (e.g., 1,3-dihydro-2H-isoindol-2-yl), 4,5,6,7-tetrahydro-1-benzofuranyl (e.g., 4,5,6,7-tetrahydro-1-benzofuran-3-yl), 4,5,6,7-tetrahydro-1-benzothienyl (e.g., 4,5,6,7-tetrahydro-1-benzothiophen-3-yl), indanyl (e.g., indan-5-yl), chromenyl (e.g., 4H-chromen-2-yl, 2H-chromen-3-yl), dihydroisoquinolinyl (e.g., 1,2-dihydroisoquinolin-4-yl), tetrahydroisoquinolinyl (e.g., 1,2,3,4-tetrahydroisoquinolin-4-yl), dihydrophthalazinyl (e.g., 1,4-dihydrophthalazin-4-yl), pyrazolidinyl (e.g., pyrazolidin-1-yl), tetrahydroquinolinyl (e.g., 1,2,3,4-tetrahydroquinolin-4-yl) and the like; and the like.

In the present specification, aromatic and nonaromatic heterocyclic group containing one or more nitrogen atoms in a molecule is collectively referred to as a nitrogen-containing heterocyclic group.

As the “substituent” of the “optionally substituted heterocycle”, those exemplified as the above-mentioned substituent group (a) can be mentioned.

The “heterocycle” of the “optionally substituted heterocycle” may have 1-5, preferably 1-3, of the aforementioned substituents at substitutable position(s) of the ring. When the number of the substituents is two or more, the substituents may be the same or different.

R is preferably a hydrogen atom.

In the formula [I], X is a spacer having 1 to 5 atoms in the main chain. The “main chain” of the “spacer having 1 to 5 atoms in the main chain” is a divalent straight chain connecting ring B and ring C, and the “atom number of the main chain” is counted such that the number of atoms in the main chain will be minimum. The “main chain” consists of 1 to 5 atoms selected from a carbon atom and a hetero atom (e.g., O, S, N etc.), and may be saturated or unsaturated. In addition, S may be oxidized.

Specific examples of the “spacer having 1 to 5 atoms in the main chain” include

a saturated divalent group, a divalent group wherein the bond is partly converted to an unsaturated bond and the like, wherein the straight chain has 1 to 5 atoms, such as
(1) —(CH₂)_{f1}— (f1 is an integer of 1-5),
(2) —(CH₂)_{g1}-Z¹—(CH₂)_{g2}— (g1 and g2 are the same or different and each is an integer of 0-4, the total of g1 and g2 is 0-4, and Z¹ is NH, O, S, SO or SO₂),
(3) —(CH₂)ₕ₁-Z¹—(CH₂)ₕ₂-Z²—(CH₂)ₕ₃— (h1, h2 and h3 are the same or different and each is an integer of 0-3, the total of h1, h2 and h3 is 0-3, Z¹ and Z² are each NH, O, S, SO or SO₂, provided that when h2 is 0, then at least one of Z¹ and Z² is preferably NH) and the like.

The divalent group for X may have a substituent at any position (preferably on carbon atom), and examples of the substituent include those exemplified as the above-mentioned substituent group (a).

Preferable examples of the spacer having 1 to 5 atoms in the main chain” for X include spacers represented by the following formula:

—(CH₂)ₘ—Y—(CH₂)ₙ— [m and n are each an integer of 0 to 4 (the total of m and n does not exceed 4), Y is a bond (when Y is a bond, m is not 0), —O—, —S—, —S(O)—, —S(O)₂—, —N(R¹)— (R¹ is a hydrogen atom, an optionally substituted hydrocarbon group or an optionally substituted heterocyclic group)].

Examples of the “optionally substituted hydrocarbon group” and “optionally substituted heterocyclic group” for R¹ include those exemplified as the above-mentioned “optionally substituted hydrocarbon group” and “optionally substituted heterocyclic group” for R.

Preferable examples of the “spacer having 1 to 5 atoms in the main chain” for X include —CH₂O—, —CH₂—, —CH₂CH₂—, SO₂—, —NHCOOCH₂—, —CH₂CH₂O— and —CH₂—N(CH₃)—, and —CH₂O— is particularly preferably used.

As compound (I), the following is preferable:

### (Compound IA)

A compound of the formula [I], wherein
ring A is
aromatic cyclic hydrocarbon (preferably, benzene) or 5- or 6-membered monocyclic aromatic heterocycle (preferably, pyrazole, pyridine, pyrimidine, pyrazine, triazole, thiazole, isothiazole, thiadiazole, pyridazine, thiophene, isoxazole) each optionally substituted by 1 to 3 substituents selected from
(1) a C₁₋₆ alkyl group (e.g., methyl, ethyl, propyl, isopropyl, butyl, isobutyl, sec-butyl, tert-butyl, pentyl, hexyl) optionally substituted by 1 to 3 substituents selected from
(a) a 3- to 8-membered nitrogen-containing heterocyclic group (preferably, pyrazolyl, pyrrolyl, pyridyl etc.),
(b) a halogen atom,
(c) a hydroxy group, and
(d) a C₃₋₆ cycloalkyl group (e.g., cyclopropyl, cyclobutyl, cyclopentyl, cyclohexyl, preferably cyclopropyl group),
   (2) a hydroxy group optionally substituted by a C₇₋₁₂ aralkyl group (e.g., benzyl, α-methylbenzyl, phenethyl),
   (3) a C₇₋₁₂ aralkyl group (e.g., benzyl, α-methylbenzyl, phenethyl) optionally substituted by 1 to 3 halogen atoms,
   (4) a C₆₋₁₀ aryl group (e.g., phenyl, naphthyl, preferably phenyl group) optionally substituted by 1 to 3 halogen atoms,
   (5) a halogen atom,
   (6) a carbamoyl group,
   (7) a 3- to 8-membered nitrogen-containing heterocyclic group (e.g., pyridyl),
   (8) a C₆₋₁₀ aryloxy group (e.g., phenoxy) optionally substituted by 1 to 3 halogen atoms, and
   (9) a C₁₋₆ alkoxy group (e.g., methoxy, ethoxy, propoxy, isopropoxy, butoxy, isobutoxy, sec-butoxy, tert-butoxy);
   ring B is
   aromatic cyclic hydrocarbon (preferably, benzene) or 5- or 6-membered nitrogen-containing heterocycle (preferably, pyridine, pyrazole, morpholine, piperidine, pyrrolidine) optionally substituted by 1 to 3 substituents selected from
   (1) a C₁₋₆ alkyl group (e.g., methyl, ethyl, propyl, isopropyl, butyl, isobutyl, sec-butyl, tert-butyl, pentyl, hexyl), and
   (2) a C₁₋₆ alkoxy group (e.g., methoxy, ethoxy, propoxy, isopropoxy, butoxy, isobutoxy, sec-butoxy, tert-butoxy);
   ring C is
   a 6-membered aromatic ring (preferably, benzene, pyridine, pyrimidine) optionally substituted by 1 to 3 substituents selected from
   (1) a C₁₋₆ alkyl group (e.g., methyl, ethyl, propyl, isopropyl, butyl, isobutyl, sec-butyl, tert-butyl, pentyl, hexyl) optionally substituted by 1 to 3 halogen atoms,
   (2) a 3- to 8-membered nitrogen-containing heterocyclic group (e.g., isoxazolyl, pyrazolyl, oxadiazolyl) optionally substituted by 1 to 3 C₁₋₆ alkyl groups (e.g., methyl, ethyl, propyl, isopropyl, butyl, isobutyl, sec-butyl, tert-butyl, pentyl, hexyl),
   (3) a C₁₋₆ alkoxy group (e.g., methoxy, ethoxy, propoxy, isopropoxy, butoxy, isobutoxy, sec-butoxy, tert-butoxy) optionally substituted by 1 to 3 halogen atoms,
   (4) a C₁₋₆ alkoxy-carbonyl group (e.g., methoxycarbonyl, ethoxycarbonyl, propoxycarbonyl, isopropoxycarbonyl, butoxycarbonyl, isobutoxycarbonyl, tert-butoxycarbonyl),
   (5) an oxo group,
   (6) a halogen atom, and
   (7) a cyano group;
   R is a hydrogen atom; and

### X is —CH₂O—.

The “optionally further substituted pyrazole ring” for ring A′ in the formula [II] means a pyrazole ring optionally having 1-3 substituents other than R² on the ring.

As the substituent that the pyrazole ring may have, those exemplified as the above-mentioned substituent group (a) can be mentioned.

Ring A′ is preferably a pyrazole ring.

As the “ring” of the “optionally substituted ring” for ring B′ in the formula [II], from those exemplified as the “ring” of the “optionally substituted ring” for ring B in the above-mentioned formula [I], those other than furan ring can be mentioned, with preference given to benzene, piperidine, morpholine, pyrrolidine and pyridine.

The “ring” of the “optionally substituted ring” for ring B′ may have 1 to 3 substituents at substitutable position(s), and when the number of the substituents is two or more, the substituents may be the same or different. As such substituent, those exemplified as the above-mentioned substituent group (a) can be mentioned.

Ring B′ is preferably unsubstituted benzene, piperidine, morpholine, pyrrolidine or pyridine.

In the formula [II], one of ring B′ and ring C is heterocycle.

As the “optionally substituted hydrocarbon group” for R² in the formula [II], those exemplified as the “optionally substituted hydrocarbon group” for R in the above-mentioned formula [I] except a methyl group can be mentioned.

As the “optionally substituted heterocyclic group” for R² in the formula [II], those exemplified as the “optionally substituted heterocyclic group” for R in the above-mentioned formula [I] can be mentioned.

R² is preferably

(1) an optionally substituted C₁₋₆ alkyl group;
(2) an optionally substituted C₇₋₁₂ aralkyl group;
(3) an optionally substituted C₆₋₁₀ aryl group; or
(4) an optionally substituted 5- or 6-membered nitrogen-containing heterocyclic group;
more preferably,
(1) a C₁₋₆ alkyl group (e.g., methyl, ethyl, propyl, isopropyl, butyl, isobutyl, sec-butyl, tert-butyl, pentyl, hexyl, provided that the methyl group always has substituent(s)) optionally substituted by 1 to 3 substituents selected from
(a) a 5- or 6-membered nitrogen-containing heterocyclic group (preferably, 5- or 6-membered aromatic heterocyclic group (e.g., pyrrolyl, pyridyl)),
(b) a halogen atom,
(c) a hydroxy group, and
(d) a C₃₋₆ cycloalkyl group (e.g., cyclopropyl, cyclobutyl, cyclopentyl, cyclohexyl, preferably cyclopropyl);
   (2) a C₇₋₁₂ aralkyl group (e.g., benzyl, α-methylbenzyl, phenethyl) optionally substituted by 1 to 3 halogen atoms,
   (3) a C₆₋₁₀ aryl group (e.g., phenyl, naphthyl, preferably a phenyl group) optionally substituted by 1 to 3 halogen atoms, or
   (4) a 5- or 6-membered nitrogen-containing heterocyclic group (preferably, a 5- or 6-membered aromatic heterocyclic group (e.g., pyridyl)).

In the formula [II], Y¹ is represented by the following formula:

—C(R³) (R⁴)—X¹— (R³ and R⁴ are each independently a hydrogen atom, an optionally substituted hydrocarbon group, an optionally substituted heterocyclic group, an optionally substituted hydroxy group, an optionally substituted amino group, an optionally substituted mercapto group, a cyano group, a nitro group, an optionally substituted acyl group or a halogen atom, and X¹ is a spacer having 1 to 4 atoms in the main chain).

As the “optionally substituted hydrocarbon group” and “optionally substituted heterocyclic group”, those similar to the groups exemplified as the “optionally substituted hydrocarbon group” and the “optionally substituted heterocyclic group” for R in the above-mentioned formula [I] can be mentioned.

The “optionally substituted hydroxy group”, “optionally substituted amino group” and “optionally substituted mercapto group” may have 1 or 2 substituents at any substitutable position(s) and, when two substituents are present, they may be the same or different. As the substituent, the “optionally substituted hydrocarbon group” and the “optionally substituted heterocyclic group” are preferable. Here, as the “optionally substituted hydrocarbon group” and the “optionally substituted heterocyclic group”, those similar to the groups exemplified as the “optionally substituted hydrocarbon group” and the “optionally substituted heterocyclic group” for R in the above-mentioned formula [I] can be mentioned.

Examples of the “optionally substituted acyl group” include an optionally substituted hydrocarbon-carbonyl group, an optionally substituted heterocyclyl-carbonyl group, an optionally substituted hydrocarbon-sulfonyl group, an optionally substituted heterocyclyl-sulfonyl group and the like.

As the “optionally substituted hydrocarbon” of the “optionally substituted hydrocarbon-carbonyl group”, those similar to the groups exemplified as the “optionally substituted hydrocarbon group” for R in the above-mentioned formula [I] can be mentioned.

Examples of the “optionally substituted hydrocarbon-carbonyl group” include a C₁₋₈ alkyl-carbonyl group, a C₂₋₈ alkenyl-carbonyl group, a C₂₋₈ alkynyl-carbonyl group, a C₃₋₈ cycloalkyl-carbonyl group, a C₃₋₈ cycloalkenyl-carbonyl group, a C₃₋₈ cycloalkyl-C₁₋₄ alkyl-carbonyl group, a C₃₋₈ cycloalkenyl-C₁₋₄ alkyl-carbonyl group, a C₆₋₁₈ aryl-carbonyl group, a C₆₋₁₈ aryl-C₁₋₄ alkyl-carbonyl group and the like, each being optionally substituted.

Examples of the “C₁₋₈ alkyl” here include methyl, ethyl, propyl, isopropyl, butyl, isobutyl, sec-butyl, tert-butyl, pentyl, hexyl, heptyl, octyl and the like, examples of the “C₂₋₈ alkenyl” include vinyl, 1-propenyl, allyl, isopropenyl, butenyl, isobutenyl and the like, examples of the “C₂₋₈ alkynyl” include ethynyl, propargyl, 1-propynyl and the like, examples of the “C₃₋₈ cycloalkyl” include cyclopropyl, cyclobutyl, cyclopentyl, cyclohexyl and the like, examples of the “C₃₋₈ cycloalkenyl” include cyclopropenyl, cyclobutenyl, cyclopentenyl, cyclohexenyl and the like, examples of the “C₁₋₄ alkyl” include those exemplified as the above-mentioned “C₁₋₈ alkyl” having 1-4 carbon atoms and, examples of the “C₆₋₁₈ aryl” include phenyl, 1-naphthyl, 2-naphthyl, biphenylyl, 2-anthryl and the like.

As the “optionally substituted hydrocarbon” of the “optionally substituted hydrocarbon-sulfonyl group”, those similar to the groups exemplified as the “optionally substituted hydrocarbon group” for R in the above-mentioned formula [I] can be mentioned.

Examples of the “optionally substituted hydrocarbon-sulfonyl group” include a C₁₋₈ alkyl-sulfonyl group, a C₂₋₈ alkenyl-sulfonyl group, a C₂₋₈ alkynyl-sulfonyl group, a C₃₋₈ cycloalkyl-sulfonyl group, a C₃₋₈ cycloalkenyl-sulfonyl group, a C₃₋₈ cycloalkyl-C₁₋₄ alkyl-sulfonyl group, a C₃₋₈ cycloalkenyl-C₁₋₄ alkyl-sulfonyl group, a C₆₋₁₈ aryl-sulfonyl group, a C₆₋₁₈ aryl-C₁₋₄ alkyl-sulfonyl group and the like, each being optionally substituted.

As the “C₁₋₈ alkyl”, “C₂₋₈ alkenyl”, “C₂₋₈ alkynyl”, “C₃₋₈ cycloalkyl”, “C₃₋₈ cycloalkenyl”, “C₁₋₄ alkyl” and “C₆₋₁₈ aryl”, those similar to the groups exemplified as the hydrocarbon group of the above-mentioned “optionally substituted hydrocarbon group” can be mentioned.

As the “optionally substituted heterocycle” of the “optionally substituted heterocyclyl-carbonyl group” and the “optionally substituted heterocyclyl-sulfonyl group”, those similar to the groups exemplified as the “optionally substituted heterocyclic group” for R in the above-mentioned formula [I] can be mentioned.

The “optionally substituted acyl group” may have one to acceptable maximum number of substituents at any substitutable position(s). When two or more substituents are present, they may be the same or different. It optionally preferably has 1 to 5, more preferably 1 to 3, substituents.

As the “substituent” of the “optionally substituted acyl group”, those exemplified as the above-mentioned substituent group (a) can be mentioned.

R³ and R⁴ are each preferably a hydrogen atom, and R³ and R⁴ are particularly preferably hydrogen atoms at the same time.

In the formula [II], X¹ is a spacer having 1 to 4 atoms in the main chain. The “main chain” of the “spacer having 1 to 4 atoms in the main chain” means the same as the “main chain” of the above-mentioned “spacer having 1 to 5 atoms in the main chain”, and the “atom number of the main chain” is counted such that the number of atoms in the main chain will be minimum. The “main chain” consists of 1 to 4 atoms selected from a carbon atom and a hetero atom (e.g., O, S, N etc.), and may be saturated or unsaturated. Also, S may be oxidized.

As the “spacer having 1 to 4 atoms in the main chain” for X¹, a spacer having 1 to 5 atoms in the main chain for the above-mentioned X wherein the main chain has up to 4 atoms can be mentioned.

The divalent group for X¹ may have substituent(s) at any position (preferably on carbon atom), and examples of the substituent include those exemplified as the above-mentioned substituent group (a).

As the “spacer having 1 to 4 atoms in the main chain” for X¹, —O—, —CH₂— and —CH₂O— are preferable, and —O— is particularly preferably used. That is, Y¹ is preferably —CH₂O—, —CH₂CH₂— or —CH₂CH₂O—, and —CH₂O— is particularly preferably used.

As compound (II), the following is preferable:

### [Compound (IIA)]

A compound of the formula [II], wherein

ring A′ is a pyrazole ring;
ring B′ is benzene, piperidine, morpholine, pyrrolidine, or pyridine;

### R² is

(1) a C₁₋₆ alkyl group (e.g., methyl, ethyl, propyl, isopropyl, butyl, isobutyl, sec-butyl, tert-butyl, pentyl, hexyl, wherein the methyl group always has substituent(s)) optionally substituted by 1 to 3 substituents selected from
(a) a 5- or 6-membered nitrogen-containing heterocyclic group (preferably, a 5- or 6-membered aromatic heterocyclic group (e.g., pyrrolyl, pyridyl)),
(b) a halogen atom,
(c) a hydroxy group, and
(d) a C₃₋₆ cycloalkyl group (e.g., cyclopropyl, cyclobutyl, cyclopentyl, cyclohexyl, preferably cyclopropyl);
   (2) a C₇₋₁₂ aralkyl group (e.g., benzyl, α-methylbenzyl, phenethyl) optionally substituted by 1 to 3 halogen atoms,
   (3) a C₆₋₁₀ aryl group (e.g., phenyl, naphthyl, preferably a phenyl group) optionally substituted by 1 to 3 halogen atoms, or
   (4) a 5- or 6-membered nitrogen-containing heterocyclic group (preferably, a 5- or 6-membered aromatic heterocyclic group (e.g., pyridyl));
   ring C is
   a 6-membered aromatic ring (preferably, benzene, pyridine, pyrimidine) optionally substituted by 1 to 3 substituents selected from
   (1) C₁₋₆ alkyl group (e.g., methyl, ethyl, propyl, isopropyl, butyl, isobutyl, sec-butyl, tert-butyl, pentyl, hexyl) optionally substituted by 1 to 3 halogen atoms,
   (2) a 3- to 8-membered nitrogen-containing heterocyclic group (e.g., isoxazolyl, pyrazolyl, oxadiazolyl) optionally substituted by 1 to 3 C₁₋₆ alkyl groups (e.g., methyl, ethyl, propyl, isopropyl, butyl, isobutyl, sec-butyl, tert-butyl, pentyl, hexyl),
   (3) a C₁₋₆ alkoxy group (e.g., methoxy, ethoxy, propoxy, isopropoxy, butoxy, isobutoxy, sec-butoxy, tert-butoxy) optionally substituted by 1 to 3 halogen atoms,
   (4) a C₁₋₆ alkoxy-carbonyl group (e.g., methoxycarbonyl, ethoxycarbonyl, propoxycarbonyl, isopropoxycarbonyl, butoxycarbonyl, isobutoxycarbonyl, tert-butoxycarbonyl),
   (5) an oxo group,
   (6) a halogen atom, and
   (7) a cyano group;
   R is a hydrogen atom; and

### Y¹ is —CH₂O—.

### [Compound IIB]

3-[(2-chlorophenoxy)methyl]-N-[1-(4-fluorobenzyl)-1H-pyrazol-4-yl]pyrrolidine-1-carboxamide (Example 21);
4-[(2-chlorophenoxy)methyl]-N-[1-(2-hydroxyethyl)-1H-pyrazol-4-yl]pyridine-2-carboxamide (Example 169);
4-[(2-chloro-5-fluorophenoxy)methyl]-N-[1-(4-fluorobenzyl)-1H-pyrazol-4-yl]pyridine-2-carboxamide (Example 198);
N-[1-(4-fluorobenzyl)-1H-pyrazol-4-yl]-4-{[2-fluoro-5-(trifluoromethyl)phenoxy]methyl}pyridine-2-carboxamide (Example 214);
4-[(2,5-dichlorophenoxy)methyl]-N-[1-(pyridin-2-ylmethyl)-1H-pyrazol-4-yl]pyridine-2-carboxamide (Example 224);
   or a salt thereof.

As the “aromatic heterocycle” of the “optionally substituted aromatic heterocycle” for ring A in the formula [III], the “aromatic heterocycle” which is one example of the “aromatic ring” of the “optionally substituted aromatic ring” for ring A in the above-mentioned formula [I] can be mentioned.

As the aromatic heterocycle, 5- or 6-membered aromatic heterocycle (e.g., 5-membered heterocycle containing, besides carbon atom, 1-4 hetero atoms selected from a nitrogen atom, an oxygen atom and a sulfur atom such as thiophene, pyrrole, oxazole, isoxazole, thiazole, isothiazole, imidazole, pyrazole, triazole, tetrazole and the like, 6-membered heterocycle containing, besides carbon atom, 1-4 hetero atoms selected from a nitrogen atom, an oxygen atom and a sulfur atom such as pyridine, pyrimidine, thiopyran, oxazin, thiazine, triazine, pyridazine, pyrazine and the like), more preferably, 5- or 6-membered nitrogen-containing aromatic heterocycle, specifically, pyrazole, pyridine, thiazole, thiadiazole are preferable, and pyridine, thiazole, thiadiazole are more preferable.

In addition, a ring wherein the above-mentioned aromatic cyclic hydrocarbon (e.g., C₆₋₁₀ arene) and the above-mentioned 5- or 6-membered aromatic heterocycle are fused is also preferable. Also, a ring wherein the same or different two 5- or 6-membered aromatic heterocycles are fused (e.g., thienopyrazine) is preferable.

The “aromatic heterocycle” of the “optionally substituted aromatic heterocycle” for ring A″ in the formula [III] may have 1 to 5 (preferably, 1 to 3) substituents at any substitutable position(s) and, when two substituents are present, they may be the same or different. As such “substituent”, those exemplified as the above-mentioned substituent group (a) can be mentioned. The substituent is preferably

(1) a C₁₋₆ alkyl group (e.g., methyl, ethyl, propyl, isopropyl, butyl, isobutyl, sec-butyl, tert-butyl, pentyl, hexyl);
(2) an aralkyl group (e.g., C₇₋₁₂ aralkyl group such as benzyl, α-methylbenzyl, phenethyl etc.) optionally substituted by 1 to 3 halogen atoms, and the like.

The “optionally substituted aromatic heterocycle” for ring A″ is preferably an optionally substituted 5- or 6-membered nitrogen-containing aromatic heterocycle (preferably, pyrazole, pyridine, thiazole, thiadiazole, more preferably, pyridine, thiazole, thiadiazole), more preferably a 5- or 6-membered nitrogen-containing aromatic heterocycle (preferably, pyrazole, pyridine, thiazole, thiadiazole, more preferably, pyridine, thiazole, thiadiazole) optionally substituted by 1 to 3 substituents selected from

(1) a C₁₋₆ alkyl group (e.g., methyl, ethyl, propyl, isopropyl, butyl, isobutyl, sec-butyl, tert-butyl, pentyl, hexyl); and
(2) a C₇₋₁₂ aralkyl group (e.g., benzyl, α-methylbenzyl, phenethyl) optionally substituted by 1 to 3 halogen atoms.

However, the “optionally substituted aromatic heterocycle” for ring A″ is not pyrazol-4-yl having a substituent at the 1-position,

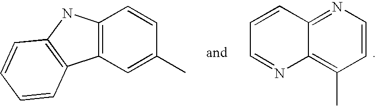

In the formula [III],

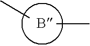

is a ring selected from

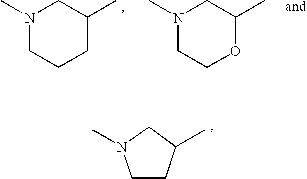

each being optionally substituted. As the “substituent” for ring B″, those exemplified as the above-mentioned substituent group (a) can be mentioned.

Ring B″ may have one to acceptable maximum number of substituents at any substitutable position(s). When two or more substituents are present, they may be the same or different. It optionally preferably has 1 to 5, more preferably 1 to 3, substituents. Moreover, these substituents are optionally substituted by substituent group (a).

Ring B″ is preferably unsubstituted.

In the formula [III], Y² is represented by the following formula:

—C(R³) (R⁴)—X²— (R³ and R⁴ are each independently a hydrogen atom, an optionally substituted hydrocarbon group, an optionally substituted heterocyclic group, an optionally substituted hydroxy group, an optionally substituted amino group, an optionally substituted mercapto group, a cyano group, a nitro group, an optionally substituted acyl group or a halogen atom, and X² is a spacer having 1 to 4 atoms in the main chain).

As the “optionally substituted hydrocarbon group”, “optionally substituted heterocyclic group”, “optionally substituted hydroxy group”, “optionally substituted amino group”, “optionally substituted mercapto group” and “optionally substituted acyl group” for R³ or R⁴ in the formula Y², those similar to the groups exemplified as the “optionally substituted hydrocarbon group”, “optionally substituted heterocyclic group”, “optionally substituted hydroxy group”, “optionally substituted amino group”, “optionally substituted mercapto group” and “optionally substituted acyl group” for R³ or R⁴ in the above-mentioned formula Y¹ can be mentioned.

In the formula Y², R³ and R⁴ are each preferably a hydrogen atom, and R³ and R⁴ are particularly preferably hydrogen atoms at the same time.

In the formula [III], X² is a spacer having 1 to 4 atoms in the main chain. The “main chain” of the “spacer having 1 to 4 atoms in the main chain” means the same as the “main chain” of the above-mentioned “spacer having 1 to 5 atoms in the main chain”, and the “atom number of the main chain” is counted such that the number of atoms in the main chain will be minimum. The “main chain” consists of 1 to 4 atoms selected from a carbon atom and a hetero atom (e.g., O, S, N etc.), and may be saturated or unsaturated. Also, S may be oxidized.

As the “spacer having 1 to 4 atoms in the main chain” for X², a spacer having 1 to 5 atoms in the main chain for the above-mentioned X wherein the main chain has up to 4 atoms can be mentioned, which is not —NH—.

The divalent group for X² may have substituent(s) at any position (preferably on carbon atom), and examples of the substituent include those exemplified as the above-mentioned substituent group (a).

As the “spacer having 1 to 4 atoms in the main chain” for X², —O—, —CH₂— and —CH₂O— are preferable, and —O— is preferably used. That is, Y² is preferably —CH₂O—, —CH₂CH₂— or —CH₂CH₂O—, and —CH₂O— is preferably used.

As compound (III), the following is preferable:

### [Compound (IIIA)]

A compound of the formula [III], wherein

ring A″ is a 5- or 6-membered nitrogen-containing aromatic heterocycle (preferably, pyrazole, pyridine, thiazole, thiadiazole, more preferably, pyridine, thiazole, thiadiazole) optionally substituted by 1 to 3 substituents selected from

(1) a C₁₋₆ alkyl group (e.g., methyl, ethyl, propyl, isopropyl, butyl, isobutyl, sec-butyl, tert-butyl, pentyl, hexyl); and
(2) a C₇₋₁₂ aralkyl group (e.g., benzyl, α-methylbenzyl, phenethyl) optionally substituted by 1 to 3 halogen atoms;

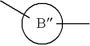

is a ring selected from

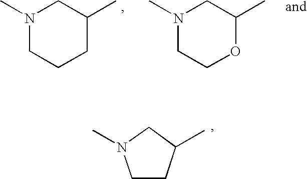

ring C is a 6-membered aromatic ring (preferably, benzene, pyridine, pyrimidine) optionally substituted by 1 to 3 substituents selected from
(1) a C₁₋₆ alkyl group (e.g., methyl, ethyl, propyl, isopropyl, butyl, isobutyl, sec-butyl, tert-butyl, pentyl, hexyl) optionally substituted by 1 to 3 halogen atoms,
(2) a 3- to 8-membered nitrogen-containing heterocyclic group (e.g., isoxazolyl, pyrazolyl, oxadiazolyl) optionally substituted 1 to 3 C₁₋₆ alkyl groups (e.g., methyl, ethyl, propyl, isopropyl, butyl, isobutyl, sec-butyl, tert-butyl, pentyl, hexyl),
(3) a C₁₋₆ alkoxy group (e.g., methoxy, ethoxy, propoxy, isopropoxy, butoxy, isobutoxy, sec-butoxy, tert-butoxy) optionally substituted by 1 to 3 halogen atoms,
(4) a C₁₋₆ alkoxy-carbonyl group (e.g., methoxycarbonyl, ethoxycarbonyl, propoxycarbonyl, isopropoxycarbonyl, butoxycarbonyl, isobutoxycarbonyl, tert-butoxycarbonyl),
(5) an oxo group,
(6) a halogen atom, and
(7) a cyano group;
R is a hydrogen atom; and

### Y² is —CH₂O—.

### [Compound IIIB]

N-(4,6-dimethylpyridin-2-yl)-2-[(2-fluorophenoxy)methyl]morpholine-4-carboxamide (Example 59);
N-(4,6-dimethylpyridin-2-yl)-2-{[2-(trifluoromethyl)phenoxy]methyl}morpholine-4-carboxamide (Example 64);
2-[(2-chloro-5-fluorophenoxy)methyl]-N-(4,6-dimethylpyridin-2-yl)morpholine-4-carboxamide (Example 78);
2-[(2-chloro-5-fluorophenoxy)methyl]-N-(6-ethylpyridin-2-yl)morpholine-4-carboxamide (Example 202);
2-[(2-chloro-5-fluorophenoxy)methyl]-N-[5-(hydroxymethyl)pyridin-2-yl]morpholine-4-carboxamide (Example 212);
   or a salt thereof.

Compound (I), compound (II) and compound (III) can also be used as salts.

The “salts” of these compounds are preferably acceptable salt as pharmaceutical products or physiologically acceptable acid addition salt. Examples of such salt include salts with inorganic acid (e.g., hydrochloric acid, phosphoric acid, hydrobromic acid, sulfuric acid etc.) or organic acid (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid etc.) and the like. When these compounds have an acidic group such as carboxylic acid and the like, for example, they may form salts with inorganic base (e.g., alkali metal or alkaline earth metal such as sodium, potassium, calcium, magnesium and the like, or ammonia etc.) or organic base (e.g., tri-C₁₋₃ alkylamine such as triethylamine and the like etc.).

The compound (I) may be used as a prodrug.

A “prodrug” of the compound (I) means a compound which is converted to the compound (I) with a reaction due to an enzyme, an gastric acid, etc. under the physiological condition in the living body, that is, a compound which is converted to the compound (I) with oxidation, reduction, hydrolysis, etc. according to an enzyme; a compound which is converted to the compound (I) by hydrolysis etc. due to gastric acid, etc. As a prodrug of compound (I), a compound obtained by subjecting an amino group in compound (I) to an acylation, alkylation or phosphorylation (e.g., a compound obtained by subjecting an amino group in compound (I) to an eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation and tert-butylation, etc.); a compound obtained by subjecting a hydroxy group in compound (I) to an acylation, alkylation, phosphorylation or boration (e.g., a compound obtained by subjecting a hydroxy group in compound (I) to an acetylation, palmitoylation, propanoylation, pivaloylation, succinylation, fumarylation, alanylation, dimethylaminomethylcarbonylation, etc.); a compound obtained by subjecting a carboxyl group in compound (I) to an esterification or amidation (e.g., a compound obtained by subjecting a carboxy group in compound (I) to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification and methylamidation, etc.) and the like, are exemplified. Any of these compounds can be produced from compound (I) by a method known per se.

In addition, the prodrug of compound (I) may be a compound, which is converted into compound (I) under the physiological conditions, as described in “Pharmaceutical Research and Development”, Vol. 7 (Drug Design), pp. 163-198 (1990), published by Hirokawa Publishing Co. In addition, compound (I) may be a hydrate.

Compound (II) and compound (III) can also be used as a prodrug, like compound (I).

When compound (I) compound (II) or compound (III) has isomers such as optical isomer, stereoisomer, positional isomer, rotational isomer and the like, and any isomers and mixtures are encompassed in the compound (I), compound (II) or compound (III). For example, when compound (I) has an optical isomer, an optical isomer separated from a racemate is also encompassed in the compound (I). Compound (II) and compound (III) as well as their isomers and mixtures are encompassed in compound (II) and compound (III), respectively. These isomers can be obtained as single products by a synthesis means or a separation means (e.g., concentration, solvent extraction, column chromatography, recrystallization and the like), and the like known per se.

The compound (I), compound (II) or compound (III) may be a crystal, and both a single crystal and crystal mixtures are encompassed in the compound (I), compound (II) or compound (III). Crystals can be produced by crystallization according to crystallization methods known per se.

The compound (I), compound (II) or compound (III) may be a solvate (e.g., hydrate etc.) or a non-solvate, both of which are encompassed in the compound (I), compound (II) or compound (III).

A compound labeled with an isotope (e.g., ³H, ¹⁴C, ³⁵S, ¹²⁵I and the like) is also encompassed in the compound (I), compound (II) or compound (III).

The production methods of compound (I) are explained in the following.

Each symbol of the compounds in the following reaction schemes is as defined above, unless otherwise specified.

In the following synthesis methods, the starting material compounds may be used in the form of salts. As such salts, those exemplified as the salts used for compounds (I), (II) and (III) can be used.

When specific production methods of the starting material compounds are not described, commercially available compounds may be easily available, or they can be produced by a method known per se or a method analogous thereto.

The compound obtained in each step can be used as a crude product (as reaction mixture) in the next reaction. In addition, the compound can be isolated from a reaction mixture according to a conventional method, and can be easily purified by a separation means such as recrystallization, distillation, chromatography and the like.

As a method of producing compound (I), for example, production method A, production method B and the like using a compound represented by the formula IV [compound (IV)] as a starting material can be mentioned.

As a method of producing a compound represented by the formula I′ [compound (I′)], for example, production method C, production method D, production method E and the like using compound (IV) or a derivative thereof, a compound represented by the formula IX [compound (IX)] or a compound represented by the formula X [compound (X)] or a derivative thereof as a starting material can be mentioned.

### Production Method A

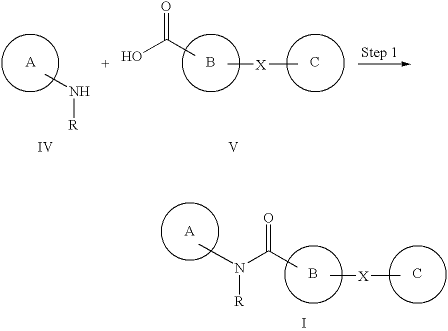

### Production Method B

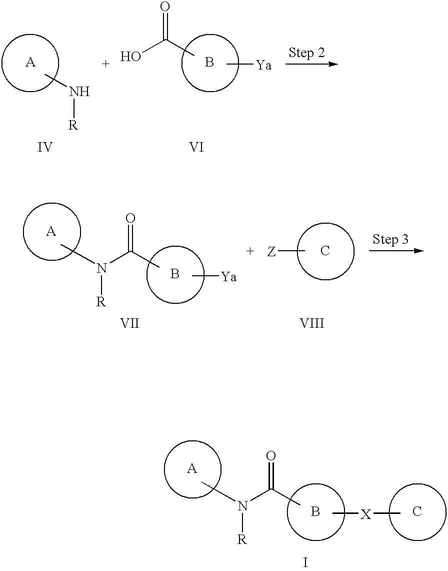

### Production Method C

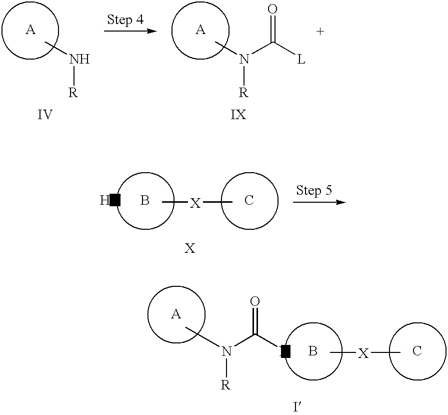

### Production Method D

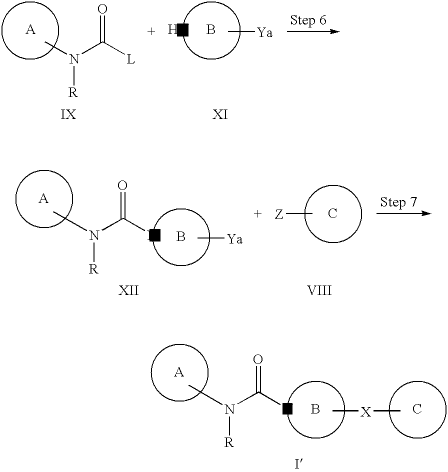

### Production Method E

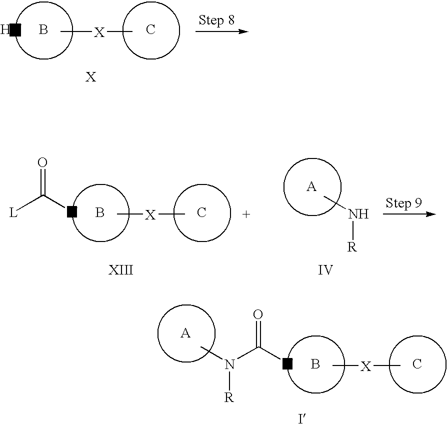

wherein L is a leaving group (e.g., a halogen, an imidazole group, a hydroxypyrrolidine-2,5-dione, optionally substituted phenoxy group or an alkoxy group); Ya is an alkyl group having 1 to 5 carbon atoms, which is optionally substituted by a halogen, a hydroxy group, an aldehyde group, an amino group, a carboxyl group, a sulfide group, an alkylsulfonyloxy group, an arylsulfonyloxy group, a boronyl group, a stanyl group, olefin, alkyne and the like; Z is an alkyl group having 1 to 4 carbon atoms, which is optionally substituted by a halogen, a hydroxy group, an aldehyde group, an amino group, a carboxyl group, a sulfide group, an alkylsulfonyloxy group, an arylsulfonyloxy group, a boronyl group, a stanyl group, olefin, alkyne and the like, and other symbols are as defined above.

Examples of the “substituent” possessed by the phenoxy group include those exemplified as the above-mentioned substituent group (a).

Examples of the method of producing compound (I) from compound (IV) or a derivative thereof and compound (V) in step 1 include (a) a method comprising condensation with a general dehydration condensation agent in the co-presence of compound (IV) and compound (V); (b) a method comprising activating carboxylic acid of compound (V) by a general activation method and reacting same with compound (IV); (c) a method comprising reacting a derivative of compound (IV) with compound (V) and the like.

### Method (a)

Examples of the dehydration condensation agent include N,N′-dicyclohexylcarbodiimide, 1-ethyl-3-(3-dimethylaminopropyl)carbodiimide, carbonyldiimidazole, N,N′-disuccinimidylcarbonate, 1H-benzotriazol-1-yloxytris(dimethylamino)phosphoniumhexafluorophosphate, O-(benzotriazol-1-yl)-N,N,N′,N′-tetramethyluroniumtetrafluoroborate and the like.

The amount of compound (V) to be used is generally 1-10 mol, preferably 1-3 mol, per 1 mol of compound (IV).

The amount of the dehydration condensation agent to be used is generally 1-10 mol, preferably 1-3 mol, per 1 mol of compound (IV).

The reaction temperature is generally 0-100° C.

The reaction time is generally 1-48 hr.

### Method (b)

Examples of a generally-known method of activating carboxylic acid include (b1) a method comprising converting to an acid anhydride with chloroformate, pivaloyl chloride and the like; (b2) a method comprising converting to an acid chloride with oxalyl chloride, thionyl chloride and the like, (b3) a method comprising converting 1-hydroxylbenzotriazole to an ester with a dehydration condensation agent and the like, and the like.

The above-mentioned method is well known in the pertinent field, and each reaction condition and the like can be appropriately set by those of ordinary skill in the art.

The amount of compound (V) to be used is generally 1-10 mol, preferably 1-3 mol, per 1 mol of compound (IV).

The reaction temperature is generally 0-100° C.

The reaction time is generally 0.5-24 hr.

### Method (c)

Examples of a method of reacting a derivative of compound (IV) with compound (V) include a method comprising heating a derivative of compound (IV) in the co-presence of compound (V).

As a derivative of compound (IV), an amine derivative and the like can be mentioned. Such derivatives can be produced by a method known per se.

The amount of compound (V) to be used is generally 1-10 mol, preferably, 1-5 mol, per 1 mol of a derivative of compound (IV).

The reaction temperature is generally 0-200° C.

The reaction time is generally 1-48 hr.

Compound (IV) can be produced by a method known per se.

Compound (V) can be produced by the below-mentioned method or a method known per se.

A method of producing compound (VII) from compound (IV) or a derivative thereof and compound (VI) in step 2 can also be performed under conditions similar to those of the above-mentioned methods (a), (b) and (c).

Compound (VI) can be produced by a method known per se.

Examples of a method of producing compound (I′) from compound (IX) or a derivative thereof and compound (X) in step 5 include a method comprising heating compound (IX) in the co-presence of compound (X).

Examples of the derivative of compound (IX) include a carbamate derivative and the like. Such derivatives can be produced by a method known per se.

The amount of compound (X) to be used is generally 1-10 mol, preferably 1-5 mol, per 1 mol of compound (IX) or a derivative thereof.

The reaction temperature is generally 0-200° C.

The reaction time is generally 1-48 hr.

Compound (X) can be produced by the below-mentioned method or a method known per se.

Examples of a method of producing compound (XII) from compound (IX) or a derivative thereof and compound (XI) in step 6 include a method comprising heating compound (IX) in the co-presence of compound (XI).

This method can be performed under conditions similar to those of the above-mentioned step 5.

Compound (XI) can be produced by a method known per se.

Examples of a method of producing compound (I′) from compound (XIII) or a derivative thereof and compound (IV) in step 9 include a method comprising heating compound (XIII) in the co-presence of compound (IV).

This method can be performed under conditions similar to those of the above-mentioned step 5.

Examples of a method of carbonylating amine of compound (IV) in step 4 include a method using a generally-known carbonylating agent.

Examples of the carbonylating agent include triphosgene analogs (e.g., diphenylcarbonate), carbonyldiimidazole/methyl iodide, trichloroethyl chloroformate, phenyl chloroformate derivative (e.g., phenylchloride carbonate) and the like.

The amount of the carbonylating agent to be used is generally 1-10 mol, preferably 1-5 mol, per 1 mol of compound (IV).

The reaction temperature is generally 0-200° C.

The reaction time is generally 1-48 hr.

As a method of carbonylating amine of compound (X) in step 8, the reaction can be performed under conditions similar to those of step 4 and using a generally-known carbonylating agent.

Examples of a method of producing compound (I) from compound (VII) or a derivative thereof and compound (VIII) in step 3 include a method comprising reacting compound (VII) with compound (VIII) in the presence of a base, an acid, a condensation agent or a transition metal.

As a derivative of compound (VII), a halogenoalkyl derivative and the like can be mentioned. Such derivatives can be produced by a method known per se.

Examples of the base include sodium hydride, potassium tert-butoxide, sodium hydroxide, lithiumhexamethyl disilazide and the like.

Examples of the acid include acetic acid, sodium cyanoborohydride, sodium triethoxyborohydride and the like.

Examples of the condensation agent include triphenylphosphine, diethylazodicarboxylate or diisobutylazodicarboxylate, N,N′-dicyclohexylcarbodiimide, 1-ethyl-3-(3-dimethylaminopropyl)carbodiimide, carbonyldiimidazole, 1H-benzotriazol-1-yloxytris(dimethylamino)phosphoniumhexafluorophosphate, O-(benzotriazol-1-yl)-N,N,N′,N′-tetramethyluroniumtetrafluoroborate and the like.

Examples of the transition metal include palladium or nickel and the like.

The amount of the base or acid to be used is generally 1-10 mol, preferably 1-3 mol, per 1 mol of compound (VII).

The amount of the condensation agent to be used is generally 1-10 mol, preferably 1-3 mol, per 1 mol of compound (VII).

The amount of the transition metal to be used is generally 0.01-3 mol, preferably 0.01-0.1 mol, per 1 mol of compound (VII).

The amount of compound (VIII) to be used is generally 1-10 mol, preferably 1-3 mol, per 1 mol of compound (VII) or a derivative thereof.

The reaction temperature is generally 0-200° C.

The reaction time is generally 1-48 hr.

Compound (VIII) can be produced by a method known per se.

As a method of producing a compound represented by compound (I′) from compound (XII) or a derivative thereof and compound (VIII) in step 7, a method similar to the above-mentioned step 3 can be mentioned.

Examples of the derivative of compound (XII) include a halogenoalkyl derivative and the like. Such derivatives can be produced by a method known per se.

compound (V) or compound (X) or a derivative thereof can be synthesized using, for example, a ring B derivative wherein Ya is bonded and a ring C derivative wherein Z is bonded and a method employed in steps 3 and 7.

In each of the aforementioned production methods of compounds (I) and (I′) as well as synthesis of the starting material compounds, when the starting compound has an amino group, a carboxyl group or a hydroxy group as a substituent, a protecting group generally used in peptide chemistry and the like may be introduced into these groups. By removing the protecting group as necessary after the reaction, the objective compound can be obtained.

Examples of the amino-protecting group include C₁₋₆ alkylcarbonyl (e.g., acetyl, ethylcarbonyl etc.), phenylcarbonyl, C₁₋₆ alkyl-oxycarbonyl (e.g., methoxycarbonyl, ethoxycarbonyl etc.), phenyloxycarbonyl, C₇₋₁₀ aralkyl-carbonyl (e.g., benzylcarbonyl etc.), trityl, phthaloyl, N,N-dimethylaminomethylene, each of which optionally has substituent(s), formyl, and the like. As these substituents, a halogen atom (e.g., fluorine, chlorine, bromine, iodine etc.), C₁₋₆ alkyl-carbonyl (e.g., methylcarbonyl, ethylcarbonyl, butylcarbonyl etc.), a nitro group and the like are used, where the number of the substituents is about 1 to 3.

Examples of the carboxyl-protecting group include C₁₋₆ alkyl (e.g., methyl, ethyl, n-propyl, i-propyl, n-butyl, tert-butyl etc.), phenyl, trityl, silyl and the like, each of which optionally has substituent(s). As these substituents, a halogen atom (e.g., fluorine, chlorine, bromine, iodine etc.), formyl, C₁₋₆ alkyl-carbonyl (e.g., acetyl, ethylcarbonyl, butylcarbonyl etc.), a nitro group and the like are used, where the number of the substituents is about 1 to 3.

Examples of the hydroxyl-protecting group include C₁₋₆ alkyl (e.g., methyl, ethyl, n-propyl, i-propyl, n-butyl, tert-butyl etc.), phenyl, C₇₋₁₀ aralkyl (e.g., benzyl etc.), formyl, C₁₋₆ alkyl-carbonyl (e.g., acetyl, ethylcarbonyl etc.), phenyloxycarbonyl, benzoyl, C₇₋₁₀ aralkyl-carbonyl (e.g., benzylcarbonyl etc.), pyranyl, furanyl, silyl and the like, each of which optionally has substituent(s). As these substituents, a halogen atom (e.g., fluorine, chlorine, bromine, iodine etc.), C₁₋₆ alkyl (e.g., methyl, ethyl, n-propyl etc.), phenyl, C₇₋₁₀ aralkyl (e.g., benzyl etc.), a nitro group and the like are used, where the number of the substituents is about 1 to 4.

For elimination of the protecting group, a method known per se or a method analogous thereto is used. For example, treatment with acid, base, ultraviolet rays, hydrazine, phenylhydrazine, sodium N-methyldithiocarbamate, tetrabutylammonium fluoride, palladium(II) acetate and the like or reduction are used.

In each of the aforementioned production methods of compounds (I) and (I′) or a salt thereof as well as synthesis of the starting material compounds, a generally-known solvent may be used for the reaction.

Examples of general solvents include ethers such as tetrahydrofuran, diethylether, 1,2-dimethoxyethane, 1,4-dioxane and the like, esters such as ethyl acetate, butyl acetate and the like, aromatic hydrocarbons such as benzene, toluene and the like, aromatic hetero ring compounds such as pyridine, lutidine and the like, amides such as N,N-dimethylformamide, N-methylpyrrolidone and the like, halides such as chloroform, methylene chloride and the like, alcohols such as methanol, ethanol, 2-propanol, 2,2-dimethylethanol and the like, hydrocarbon compounds such as hexane, heptane, petroleum ether and the like, carboxylic acids such as formic acid, acetic acid and the like, water and the like.

The solvent to be used for the reaction may be a single solvent, or a mixed solvent of 2 to 6 kinds of solvents.

In the reaction, for example, amines such as triethylamine, N,N-diisopropylamine, pyridine, N-methylmorpholine and the like, a base such as sodium hydroxide, potassium carbonate and the like may be co-present.

In the reaction, for example, an acid such as hydrochloric acid, sulfuric acid, acetic acid and the like may be co-present.

Compounds (I) and (I′) or salts thereof obtained by the foregoing methods can be isolated and purified by, for example, a general separation means such as recrystallization, distillation, chromatography and the like. The thus-obtained compounds (I) and (I′) of the present invention are in a free form, they can be converted to salts by a method known per se or a method analogous thereto (e.g., neutralization etc.). When they are obtained as salts, they can be converted to a free form or other salts by a method known per se or a method analogous thereto. When the obtained compound is a racemate, it can be separate into a d form or an l form by a general means of optical resolution.

The starting material compounds of compounds (I) and (I′) or a salt thereof can also be salts similar to those of compounds (I) and (I′), and are not particularly limited as long as they do not interfere with the reaction.

Compound (II) and compound (III) or salts thereof can be obtained by a method similar to that of compound (I) or a salt thereof.

Compound (I), compound (II) and compound (III) or a prodrug thereof (hereinafter to be also referred to as the compound of the present invention) show low toxicity (e.g., superior as pharmaceutical agents from the aspects of acute toxicity, chronic toxicity, genetic toxicity, reproductive toxicity, cardiotoxicity, drug interaction, carcinogenicity and the like), and can be administered safely as an SCD inhibitor to mammals (e.g., human, monkey, bovine, horse, swine, mouse, rat, hamster, rabbit, cat, dog, sheep, goat etc.), directly as a pharmaceutical agent, or in the form of a pharmaceutical composition upon blending with a pharmaceutically acceptable carrier known per se and the like.

Since the compound of the present invention shows an SCD inhibitory action (particularly, SCD-1 inhibitory action), it is useful as an SCD inhibitor.

In addition, the compound of the present invention can show a fatty acid desaturation inhibitory action, an insulin signal enhancing action, suppression of body weight gain and a visceral fat-decreasing action based on a promoted energy consumption, plasma and liver triglyceride lowering action, cholesterol ester and lipoprotein synthesis inhibitory action, and cholesterol efflux improving effect via ATP-binding cassette transporter A1 (ABCA1), which are afforded by an SCD inhibitory action (particularly, SCD-1 inhibitory action). Accordingly, the compound of the present invention is useful as a pharmaceutical agent based on the above-mentioned action.

Specifically, the compound of the present invention is highly useful as a prophylactic or therapeutic agent for hyperlipidemia (including hypercholesterolemia, high LDL-cholesterolemia, low HDL-cholesterolemia and hypertriglyceride(TG)mia and the like, particularly, hypertriglyceridemia), diabetes (including type 1 diabetes, type 2 diabetes, gestational diabetes, obese diabetes and the like, particularly, type 2 diabetes), obesity, abnormal lipid metabolism, fatty liver, metabolic syndrome, arteriosclerosis associated disease and fatal myocardial infarction, sudden cardiac death, nonfatal myocardial infarction, angina pectoris decubitus or effort angina pectoris, instabilization of angina pectoris, cardio- and cerebrovascular disorders (cardiovascular diseases including cerebral thrombus, cerebral embolism, cerebral hemorrhage, subarachnoid hemorrhage, TIA (transient cerebral ischemic attack; Transient ischemic attack)) and the like.

For diagnostic criteria of diabetes, Japan Diabetes Society reported new diagnostic criteria in 1999.

According to this report, diabetes is a condition showing any of a fasting blood glucose level (glucose concentration of intravenous plasma) of not less than 126 mg/dl, a 75 g oral glucose tolerance test (75 g OGTT) 2 h level (glucose concentration of intravenous plasma) of not less than 200 mg/dl, and a non-fasting blood glucose level (glucose concentration of intravenous plasma) of not less than 200 mg/dl. A condition not falling under the above-mentioned diabetes and different from “a condition showing a fasting blood glucose level (glucose concentration of intravenous plasma) of less than 110 mg/dl or a 75 g oral glucose tolerance test (75 g OGTT) 2 h level (glucose concentration of intravenous plasma) of less than 140 mg/dl” (normal type) is called a “borderline type”.

In addition, ADA (American Diabetes Association) reported new diagnostic criteria of diabetes in 1997 and WHO in 1998.

According to these reports, diabetes is a condition showing a fasting blood glucose level (glucose concentration of intravenous plasma) of not less than 126 mg/dl and a 75 g oral glucose tolerance test 2 h level (glucose concentration of intravenous plasma) of not less than 200 mg/dl.

According to the above-mentioned reports, impaired glucose tolerance is a condition showing a fasting blood glucose level (glucose concentration of intravenous plasma) of less than 126 mg/dl and a 75 g oral glucose tolerance test 2 h level (glucose concentration of intravenous plasma) of not less than 140 mg/dl and less than 200 mg/dl. According to the report of ADA, a condition showing a fasting blood glucose level (glucose concentration of intravenous plasma) of not less than 110 mg/dl and less than 126 mg/dl is called IFG (Impaired Fasting Glucose). According to the report of WHO, among the IFG (Impaired Fasting Glucose), a condition showing a 75 g oral glucose tolerance test 2 h level (glucose concentration of intravenous plasma) of less than 140 mg/dl is called IFG (Impaired Fasting Glycemia).

The compound of the present invention can be also used as an agent for the prophylaxis or treatment of diabetes, borderline type, impaired glucose tolerance, IFG (Impaired Fasting Glucose) and IFG (Impaired Fasting Glycemia), as determined according to the above-mentioned new diagnostic criteria. Moreover, the compound of the present invention can prevent progress of borderline type, impaired glucose tolerance, IFG (Impaired Fasting Glucose) or IFG (Impaired Fasting Glycemia) into diabetes.

In the present specification, SCD inhibitors and pharmaceutical agents containing the compound of the present invention are sometimes collectively referred to as “the SCD inhibitor of the present invention”.

For administration of the SCD inhibitor of the present invention, the compound of the present invention, which is the active ingredient, may be used as bulk. Generally, however, it is administered in the form of a pharmaceutical preparation formulated according to a conventional method using an appropriate amount of a carrier for preparation, such as an excipient (e.g., calcium carbonate, kaolin, sodium hydrogen carbonate, lactose, starches, crystalline cellulose, talc, granulated sugar, porous substance etc.), binder (e.g., dextrin, rubbers, alcoholized starch, gelatin, hydroxypropylcellulose, hydroxypropylmethylcellulose, pullulan etc.), a disintegrant (e.g., carboxymethylcellulose calcium, croscarmellose sodium, crospovidone, low-substituted hydroxypropylcellulose, partially pregelatinized starch etc.), a lubricant (e.g., magnesium stearate, calcium stearate, talc, starch, sodium benzoate etc.), a colorant (e.g., tar pigment, caramel, diiron trioxide, titanium oxide, riboflavins etc.), a corrigent (e.g., sweeteners, flavor etc.), a stabilizer (e.g., sodium sulfite etc.), a preservative (e.g., parabens, sorbic acid etc.) and the like.

The SCD inhibitor of the present invention appropriately contains the compound of the present invention in an amount effective for the treatment or prophylaxis of a disease. The content of the compound of the present invention in the SCD inhibitor is generally 0.1 to 100 wt % of the whole preparation. The SCD inhibitor of the present invention may contain pharmaceutical components other than the compound of the present invention as an active ingredient. Such component is not particularly limited as long as the object of the present invention can be achieved, and can be appropriately used at a suitable blending ratio.

Specific examples of the dosage form include tablet (including sugar-coated tablet, film-coated tablet, orally disintegrating tablet), film (including orally disintegrable film), pill, capsule, granule, fine granules, powder, syrup, emulsion, suspension, injection, sustained-release injection, inhalant, ointment and the like. These preparations are prepared according to a conventional method (e.g., the method described in the Japanese Pharmacopoeia etc.).

Specifically, as a production method of a tablet, the compound of the present invention as it is, or a homogeneous blend of the compound and an excipient, a binder, a disintegrant or any other suitable additive, and the like, is granulated by a suitable method, a lubricant and the like are added, and the mixture is compression formed; the compound of the present invention as it is, or a homogeneous blend of the compound and an excipient, a binder, a disintegrant or any other suitable additive, and the like, is directly compression formed; or granules produced in advance, or a homogeneous blend of the granules and a suitable additive, are(is) compression formed. In addition, the agent can contain a colorant, a corrigent and the like as necessary. Moreover, the agent can be coated with a suitable coating agent.

As a production method of an injection, a given amount of the compound of the present invention is dissolved, suspended or emulsified in water for injection, saline, Ringer's solution and the like to give an aqueous agent, a given amount is generally dissolved, suspended or emulsified in vegetable oil and the like to give a nonaqueous agent, or a given amount the compound of the present invention is tightly sealed in a container for injection.

As a carrier for oral preparation, a substance conventionally used in the pharmaceutical field such as starch, mannitol, crystalline cellulose, carboxymethylcellulose sodium and the like is used. Examples of the injectable carrier include distilled water, saline, glucose solution, transfusion and the like. In addition, additives generally used for preparations can also be added as appropriate.

Moreover, the SCD inhibitor of the present invention can also be used as a sustained-release preparation. The sustained-release preparation can be produced by directly using a microcapsule (e.g., microsphere.microcapsule, microparticle etc.) produced, for example, by in-water drying method (o/w method, w/o/w method etc.), phase separation method, spray drying method or a method analogous thereto may be administered as it is, or said microcapsule, or a pharmaceutical composition in the form of sphere, needle, pellet, film or a cream as a starting material may be formulated into various dosage forms and administered. Examples of the dosage form include parenteral agent (e.g., intramuscular, subcutaneous, intravenous, intraperitoneal or organ injection or implant and the like; transmucosal agent for nasal cavity, rectum, uterus and the like, etc.), oral preparation (e.g., hard capsule, soft capsule, granule, powder, suspension etc.) and the like.

When the sustained-release preparation is an injection, the microcapsule is processed with a dispersing agent (e.g., surfactant such as Tween 80, HCO-60 and the like; polysaccharides such as carboxymethylcellulose, sodium alginate, sodium hyaluronate and the like; protamine sulfate, polyethylene glycol etc.), preservative (e.g., methylparaben, propylparaben etc.), an isotonicity agent (e.g., sodium chloride, mannitol, sorbitol, glucose etc.), a topical anesthetic (e.g., xylocaine hydrochloride, chlorobutanol etc.) and the like to give an aqueous suspension, or dispersed in a vegetable oil (e.g., sesame oil, corn oil etc.) or a mixture thereof with phospholipid (e.g., lecithin etc.), or medium-chain triglyceride (e.g., miglyol 812 etc.) to give an oil suspension as a sustained-release injection.

When the sustained-release preparation is a microcapsule, its average particle size is about 0.1 to about 300 μm, preferably about 1 to about 150 μm, more preferably about 2 to about 100 μm.

To formulate an aseptic microcapsule preparation, a method comprising sterilizing the whole production steps, a method comprising sterilization with γ rays, a method comprising addition of a preservative and the like can be nonlimitatively mentioned.

While the dose of the SCD inhibitor of the present invention varies depending on the administration route, symptom, age or body weight of patients and the like, it is, for example, 0.1-500 mg/day, preferably 1-100 mg/day, as the compound of the present invention for oral administration to an adult patient as an agent for the prophylaxis or treatment of hyperlipidemia, diabetes, obesity, abnormal lipid metabolism, fatty liver, metabolic syndrome, arteriosclerosis associated disease, cardiovascular disease and the like, which is desirably administered in one to several portions a day. The administration route may be any of oral and parenteral.

Moreover, while the dose of a sustained-release preparation as an example of the SCD inhibitor of the present invention also varies depending on the administration route, symptom, age or body weight of patients and the like, as well as duration of release and the like, it is not particularly limited as long as the effective concentration of the active ingredient can be maintained in the body. The administration frequency is once a day to 3 days or once a week to 3 months and the like, which can be appropriately determined according to the situation.

The SCD inhibitor of the present invention can be used concurrently with other drug treatment, hormone replacement therapy or surgical method. Accordingly, the present invention also provides a combination drug using an SCD inhibitor and other drug or various treatment methods in combination.

Examples of the drug that can be used concurrently with an SCD inhibitor in the combination drug of the present invention (hereinafter sometimes to be abbreviated as a concomitant drug) include a drug having a blood lipid improving effect other than SCD inhibitors, a drug showing a prophylactic or therapeutic effect on any of various diseases that promote arteriosclerosis or ischemic cardiac diseases, and the like.

Examples of the drug having a blood lipid improving effect other than SCD inhibitors include HMG-CoA reductase inhibitors, fibrate compounds, squalene synthase inhibitors, ACAT (Acyl-CoA: cholesterol acyltransferase) inhibitors, cholesterol absorption suppressive drug ezetimibe and the like.

Examples of the HMG-CoA reductase inhibitor include pravastatin, simvastatin, lovastatin, atorvastatin, fluvastatin, pitavastatin, rosuvastatin or a salt thereof (e.g., sodium salt etc.) and the like.

Examples of the fibrate compound include bezafibrate, beclobrate, binifibrate, ciprofibrate, clinofibrate, clofibrate, clofibric acid, etofibrate, fenofibrate, gemfibrozil, nicofibrate, pirifibrate, ronifibrate, simfibrate, theofibrate and the like.

Examples of the squalene synthase inhibitor include the compound described in WO97/10224, for example, N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid and the like.

Examples of the ACAT inhibitor include avasimibe, eflucimibe, pactimibe and the like.

Besides the above-mentioned, examples of the drug having a blood lipid improving effect include, but are not limited to, anion exchange resin (e.g., colestyramine etc.), probucol, nicotinic acid drug (e.g., nicomol, niceritrol etc.), fish oil preparation (e.g., ethyl icosapentate, ethyl docosahexaenoate, Omacor or a mixture thereof), phytosterol (e.g., soysterol, γ-oryzanol etc.) and the like.

As the hormone replacement therapy, thyroid hormone or estrogen replacement therapy and the like can be mentioned.

As the surgical method, intervention treatments such as LDL apheresis, percutaneous transluminal coronary angioplasty, percutaneous coronary recanalization, stenting and the like, and the like can be mentioned nonlimitatively.

On the other hand, as various diseases, pathology and factor that promote arteriosclerosis and ischemic cardiac diseases, there are known hypertension, diabetes, obesity, thrombotic tendency, autoimmune hyperlipidemia, inflammatory diseases, infectious diseases and the like. Examples of the drug having a prophylactic or therapeutic effect on any of them include, but are not limited to a therapeutic drug for hypertension, a therapeutic drug for diabetes, an anti-obesity drug, an antithrombotic, an anti-inflammatory drug, an anti-rheumatic drug, an antibacterial agents, an antifungal agent, an antivirus drug, an antiallergic agent, an anti-angiopathic drug and the like.

Examples of the therapeutic drug for diabetes include PPAR modulator, insulin secretagogue, biguanide, insulin preparation, α-glucosidase inhibitor, β3 agonist and the like.

Examples of the PPAR modulator include PPARγ agonist such as glitazone drug and glitazar drug and the like, and a PPARδ agonist. For example, troglitazone, pioglitazone, rosiglitazone, muraglitazar, GW501516 and the like can be mentioned.

Examples of the insulin secretagogue include sulfonylurea drug. Specific examples of the sulfonylurea drug include tolbutamide, chlorpropamide, tolazamide, acetohexamide, glyclopyramide and an ammonium salt thereof, glibenclamide, gliclazide, 1-butyl-3-metanilylurea, carbutamide, glibornuride, glipizide, gliquidone, glisoxepid, glybuthiazole, glybuzole, glyhexamide, glymidine, glypinamide, phenbutamide, tolylcyclamide, glimepiride and the like can be mentioned. Besides these, examples of the insulin secretagogue include N-[[4-(1-methylethyl)cyclohexyl]carbonyl]-D-phenylalanine [nateglinide], (2S)-2-benzyl-3-(cis-hexahydro-2-isoindolinylcarbonyl)propionic acid calcium dihydrate [mitiglinide], repaglinide, GLP (Glucagon-like peptide)-1, GLP-1(7-36)-amide, V8-GLP-1(LY-307161), exendin-4 (AC-2993), DPP-IV inhibitor “DPP-728-A, saxagliptin, vildagliptin, sitagliptin”, V-411, JT-608 and the like.

Examples of the biguanide include phenformin, metformin, buformin and the like.

Examples of the insulin preparation include animal insulin preparations extracted from the bovine or swine pancreas; semisynthetic human insulin enzymatically synthesized from the insulin extracted from the swine pancreas; human insulin synthesized by genetic engineering using Escherichia coli or yeast and the like. As the insulin, zinc insulin containing 0.45-0.9 (w/w) % of zinc; protamine zinc insulin produced from zinc chloride, protamine sulfate and insulin and the like are also used. Moreover, insulin may be a fragment or derivative thereof (e.g., INS-1 etc.). Insulin includes various types such as ultrafast-acting, fast-acting, biphasic, intermediate, long-acting and the like, which can be appropriately selected according to the pathology of the patient.

Examples of the α-glucosidase inhibitor include acarbose, voglibose, miglitol, emiglitate and the like.

Examples of the β3 agonist (β3 adrenoceptor agonist) include SR-58611-A, SB-226552, AZ40140 and the like.

Besides the above-mentioned, examples of the therapeutic drug for diabetes include ergoset, pramlintide, leptin, BAY-27-9955, T-1095 and the like.

Examples of the anti-obesity drug include lipase inhibitors, a melanin coagulation hormone receptor antagonist and a cannabinoid receptor antagonist as anorectic drugs, and the like.

Examples of the lipase inhibitor include orlistat, ATL-962 and the like. Examples of the anorectic drug include dexfenfluramin, fluoxetine, sibutramine, biamine, rimonabant and the like.

Examples of the therapeutic drug for hypertension include angiotensin converting enzyme inhibitor, calcium antagonist, potassium channel opener, angiotensin II antagonist, rennin inhibitor, diuretic and the like.

Examples of the angiotensin converting enzyme inhibitor include captopril, enalapril, alacepril, delapril, ramipril, lisinopril, imidapril, benazepril, ceronapril, cilazapril, enalaprilat, fosinopril, moveltopril, perindopril, quinapril, spirapril, temocapril, trandolapril, manidipine and the like.

Examples of the calcium antagonist include nifedipine, amlodipine, efonidipine, nicardipine and the like.

Examples of the rennin inhibitor include aliskiren and the like.

Examples of the potassium channel opener include levcromakalim, L-27152, AL 0671, NIP-121 and the like.

Examples of the angiotensin II antagonist include losartan, candesartan cilexetil, valsartan, irbesartan, (5-methyl-2-oxo-1,3-dioxolen-4-yl)methyl 4-(1-hydroxy-1-methylethyl)-2-propyl-1-[2′-(1H-tetrazol-5-yl)biphenyl-4-ylmethyl]imidazole-5-carboxylate (CS-866), E4177 and the like.

Examples of the diuretic include xanthine derivative preparation, thiazide preparation, antialdosterone preparation, carbonic anhydrase inhibitors, chlorobenzenesulfonamide agent and the like.

Examples of the xanthine derivative preparation include theobromine sodium salicylate, theobromine calcium salicylate and the like.

Examples of the thiazide preparation include ethiazide, cyclopenthiazide, trichloromethyazide, hydrochlorothiazide, hydroflumethiazide, benzylhydrochlorothiazide, penflutizide, polythiazide, methyclothiazide and the like.

Examples of the antialdosterone preparation include spironolactone, triamterene and the like.

Examples of the carbonic anhydrase inhibitor include acetazolamide and the like.

Examples of the chlorobenzenesulfonamide agent include chlortalidone, mefruside, indapamide and the like.

Besides the above, examples of the diuretic include azosemide, isosorbide, ethacrynic acid, piretanide, bumetanide, furosemide and the like.

Examples of the antithrombotic include heparin, warfarin, anti-thrombin drug, thrombolytic agent, platelet aggregation inhibitor and the like.

Examples of the heparin include heparin sodium, heparin calcium, dalteparin sodium and the like.

Examples of the warfarin include warfarin potassium and the like.

Examples of the anti-thrombin drug include aragatroban and the like.

Examples of the thrombolytic agent include urokinase, tisokinase, alteplase, nateplase, monteplase, pamiteplase and the like.

Examples of the platelet aggregation inhibitor include ticlopidine hydrochloride, cilostazol, ethyl icosapentate, sarpogrelate hydrochloride and the like.

Examples of the anti-inflammatory drug include non-steroidal antiphlogistic analgetics which are cyclooxygenase(COX) inhibitors (e.g., salicylic acid drug such as various aspirins and the like, anthranilic drug such as mefenamic acid, flufenamic acid and the like, indoleacetic acid drug such as indomethacin, sulindac, acemetacin and the like, phenylacetic acid drug such as diclofenac, fenbufen and the like, propionic drug such as ibuprofen, ketoprofen, loxoprofen, naproxen, tiaprofen and the like, oxicam drug such as piroxicam, tenoxicam, ampiroxicam and the like, pyrazolone drug such as ketophenylbutazone and the like etc.), anti-cytokine drugs (e.g., anti-cytokine antibody such as anti-TNF-α antibody, anti-IL-6 antibody and the like, antisense oligonucleotide of cytokine gene, cytokine binding protein-1 etc.), and the like.

Examples of the anti-rheumatic drug include gold preparation such as gold sodium thiomalate, auranofin and the like, penicillamine drug such as bucillamine, penicillamine and the like, lobenzarit drug such as lobenzarit disodium and the like, acritat, salazosulfapyridine, methotrexate, mizoribine, cyclosporine, azathiopurine, cyclophosphamide, prednisolone farnesylate and the like.

Examples of the antibacterial agents include penicillin antibiotics (e.g., sawacillin, pasetocin, yamacillin, bacacil, viccillin, pentrex etc.), cephem antibiotics (e.g., keflex, kefral, cefzon, tomiron, cefspan, pansporin etc.), macrolide antibiotics (e.g., erythrosine, clarith, klaricid, rulid, josamycin etc.), tetracycline antibiotics (e.g., minomycin, vibramycin, hydramycin, ledermycin etc.), fosfomycin antibiotics (e.g., fosmicin, eukocin etc.), aminoglycoside antibiotics (e.g., kanamycin etc.), new quinolone antibacterial agents (e.g., cravat, tarivid, baccidal, tosuxacin, ozex etc.) and the like.

Examples of the antifungal agent include polyene antifungal agents (e.g., trichomycin, amphotericin B, nystatin etc.), imidazole antifungal agents (e.g., econazole, miconazole, clotrimazole etc.), triazole antifungal agents (e.g., fluconazole, itraconazole, fluconazole etc.), allylamine antifungal agents (e.g., butenafine, terbinafine hydrochloride etc.), flucytosine(5-FC) antifungal agents (e.g., flucytosine etc.) and the like.

Examples of the antivirus drug include nucleic acid synthesis inhibiting antivirus drugs (e.g., acyclovir, gancyclovir, vidarabine, foscarnet, zidovudine, lamivudine, didanosine etc.), intracellular entry suppressing antivirus drugs (e.g., amantadine, zanamivir, oseltamivir etc.), host infection defending ability enhancing antivirus drugs (e.g., interferon, isoprinosine etc.) and the like.

Examples of the antiallergic agent include anti-histamic antiallergic agents (e.g., ketotifen, azelastine, oxatomide, mequitazine, epinastine hydrochloride, terfenadine etc.), non-anti-histamic antiallergic agents (e.g., ozagrel hydrochloride, sodium cromoglycate, tranilast, repirinast, amlexanox etc.) and the like.

Examples of the anti-angiopathic drug include cilostazol, abciximab and the like.

The administration mode of an SCD inhibitor and a concomitant drug to be used in the present invention is not particularly limited, and the SCD inhibitor and the concomitant drug may be combined on administration. Examples of such administration mode include the following:

(1) administration of a single preparation obtained by simultaneously processing the SCD inhibitor and the concomitant drug (so-called combination agent), (2) simultaneous administration of two kinds of preparations of the SCD inhibitor and the concomitant drug, which have been separately produced, by the same administration route, (3) administration of two kinds of preparations of the SCD inhibitor and the concomitant drug, which have been separately produced, by the same administration route in a staggered manner, (4) simultaneous administration of two kinds of preparations of the SCD inhibitor and the concomitant drug, which have been separately produced, by different administration routes, (5) administration of two kinds of preparations of the SCD inhibitor and the concomitant drug, which have been separately produced, by different administration routes in a staggered manner (e.g., administration in the order of the SCD inhibitor and the concomitant drug, or in the reverse order) and the like.

The “concurrent use of an SCD inhibitor and a concomitant drug” in the present invention means, for example, concurrent use of the both drugs in any of the above-mentioned administration modes, and an “agent obtained by combining an SCD inhibitor with a concomitant drug” means any agent formulated for a concurrent use of the both drugs in any of the above-mentioned administration modes.

The dose of the concomitant drug can be appropriately determined based on the dose employed clinically. The mixing ratio of the SCD inhibitor and the concomitant drug can be appropriately determined according to the kind of the concomitant drug, subject of administration, administration route, target disease, symptom, combination and the like. For example, for administration of a HMG-CoA reductase inhibitor as a concomitant drug to human, 0.01 to 100 parts by weight of the SCD inhibitor is used per 1 part by weight of the HMG-CoA reductase inhibitor.

### EXAMPLES

The present invention is explained in detail in the following by referring to Reference Examples, Examples, Experimental Examples and Formulation Examples, which are mere exemplifications and do not limit the present invention. In addition, the present invention may be modified without departing from the scope of the invention.

The ¹H-NMR spectrum was measured by Varian Gemini 200 (200 MHz) or 300 (300 MHz) or BRUKER AVANCE300 (300 MHz) spectrometer using tetramethylsilane as the internal standard, and all δ values are shown in ppm. Unless otherwise specified, the numerical value shown for mixed solvent is a volume mixing ratio of each solvent. Unless otherwise specified, % means wt %. Unless otherwise specified, the ratio of elution solvents used for silica gel chromatography is a volume ratio. In the Examples, room temperature (ambient temperature) means a temperature of from about 20° C. to about 30° C.

Each symbol in the Examples means the following.

DMSO: dimethyl sulfoxide, CDCl₃: deuterated chloroform, s: singlet, d: doublet, t: triplet, q: quartet, dd: double doublet, dt: double triplet, m: multiplet, br: broad, J: coupling constant

LC/MS analyses in the Examples were performed under the following conditions.

measurement device: Waters LC/MS system
HPLC unit: Agilent HP1100
MS unit: Micromass ZMD
column: CAPCELL PAK c18UG120 S-3 μm, 1.5×35 mm (manufactured by Shiseido)
solvent: SOLUTION A; 0.05% aqueous trifluoroacetic acid solution, SOLUTION B; 0.04% trifluoroacetic acid acetonitrile solution
gradient cycle: 0 min (SOLUTION A/SOLUTION B=90/10), 2.00 min (SOLUTION A/SOLUTION B=5/95), 2.75 min (SOLUTION A/SOLUTION B=5/95), 2.76 min (SOLUTION A/SOLUTION B=90/10), 3.60 min (SOLUTION A/SOLUTION B=90/10)
injection volume: 2 μL, flow rate: 0.5 mL/min, detection method: UV 220 nm
MS conditions ionization method: ESI

Purification by preparative HLPC in the Examples were performed under the following conditions.

equipment: GILSON high through-put purification system
column: YMC CombiPrep ODS-A S-5 μm, 50×20 mm or YMC CombiPrep Hydrosphere C18 S-5 μm, 50×20 mm
solvent: SOLUTION A; 0.1% aqueous trifluoroacetic acid solution, SOLUTION B; 0.1% trifluoroacetic acid acetonitrile solution
gradient cycle: 0 min (SOLUTION A/SOLUTION B=95/5), 1.00 min (SOLUTION A/SOLUTION B=95/5), 5.20 min (SOLUTION A/SOLUTION B=5/95), 6.40 min (SOLUTION A/SOLUTION B=5/95), 6.50 min (SOLUTION A/SOLUTION B=95/5), 6.60 min (SOLUTION A/SOLUTION B=95/5), or 0 min (SOLUTION A/SOLUTION B=98/2), 1.00 min (SOLUTION A/SOLUTION B=98/2), 5.00 min (SOLUTION A/SOLUTION B=0/100), 6.40 min (SOLUTION A/SOLUTION B=0/100), 6.50 min (SOLUTION A/SOLUTION B=98/2), 6.60 min (SOLUTION A/SOLUTION B=98/2)
flow rate: 20 mL/min, detection method: UV 220 nm

### Example 1

### 3-[(2,5-dichlorophenoxy)methyl]-N-[4-(1H-pyrazol-1-ylmethyl)phenyl]benzamide

### (1) methyl 3-[(2,5-dichlorophenoxy)methyl]benzoate

A solution of methyl 3-(bromomethyl)benzoate (10 g), 2,5-dichlorophenol (7.1 g) and potassium carbonate (7.8 g) in N,N-dimethylformamide (150 mL) was stirred at 50° C. overnight. After cooling, water (50 mL) was added, and the mixture was extracted with ethyl acetate. The organic layer was washed with saturated brine and dried over sodium sulfate. The solvent was evaporated under reduced pressure, and the residue was purified by silica gel column (20% ethyl acetate/hexane to 30% ethyl acetate/hexane) to give a white solid (13.5 g, 99%).

¹H NMR (300 MHz, DMSO-d₆) δ ppm 3.93 (s, 3H) 5.16 (s, 2H) 6.91 (dd, J=2.4, 8.4 Hz, 1H) 6.95 (d, J=2.1 Hz, 1H) 7.29 (d, J=8.4 Hz, 1H) 7.48 (t, J=7.8 Hz, 1H) 7.68 (d, J=7.8 Hz, 1H) 8.01 (d, J=7.8 Hz, 1H) 8.12 (brs, 1H)

### (2) 3-[(2,5-dichlorophenoxy)methyl]benzoic acid

To a solution of methyl 3-[(2,5-dichlorophenoxy)methyl]benzoate (13.5 g), obtained in the above-mentioned reaction, in methanol (300 mL) was added 4N aqueous sodium hydroxide solution (100 mL), and the mixture was stirred at 50° C. overnight. After cooling, 6N hydrochloric acid (80 mL) was added, and the resulting precipitate was collected by filtration and dried to give the object product (11.3 g, 88%) as a white solid.

¹H NMR (300 MHz, DMSO-d₆) δ ppm 5.33 (s, 2H) 7.05 (dd, J=2.7, 8.7 Hz, 2H) 7.37 (d, J=2.4 Hz, 1H) 7.47-7.58 (m, 2H) 7.70 (d, J=7.5 Hz, 1H) 7.92 (d, J=7.5 Hz, 1H) 8.06 (s, 1H)

### (3) 3-[(2,5-dichlorophenoxy)methyl]-N-[4-(1H-pyrazol-1-ylmethyl)phenyl]benzamide

A solution of 3-[(2,5-dichlorophenoxy)methyl]benzoic acid (2.97 g) obtained in the above-mentioned reaction, 4-(1H-pyrazol-1-ylmethyl)aniline (1.73 g), 1-ethyl-3-(3-dimethylaminopropyl)carbodiimide hydrochloride (2.3 g) and hydroxybenzotriazole hydrate (1.8 g) in N,N-dimethylformamide (80 mL) was stirred at room temperature overnight. Ethyl acetate was added, the mixture was washed 3 times with saturated aqueous sodium hydrogen carbonate solution, and the organic layer was dried over sodium sulfate. The solvent was evaporated under reduced pressure, and ethyl acetate/hexane was added. The resulting precipitate was collected by filtration, and recrystallized from ethyl acetate/hexane to give the object product (3.34 g, 74%) as a white solid.

¹H NMR (300 MHz, DMSO-d₆) δ ppm 5.30 (s, 2H) 5.34 (s, 2H) 6.27 (t, J=2.07 Hz, 1H) 7.06 (dd, J=2.35, 8.57 Hz, 1H) 7.23 (d, J=8.67 Hz, 2H) 7.40 (d, J=2.45 Hz, 1H) 7.46 (d, J=1.32 Hz, 1H) 7.49 (d, J=8.48 Hz, 1H) 7.54-7.62 (m, 1H) 7.65-7.76 (m, 3H) 7.78-7.82 (m, 1H) 7.90-7.96 (m, 1H) 7.99-8.04 (m, 1H) 10.34 (s, 1H)

### Example 2

### 3-[(2,5-dichlorophenoxy)methyl]-N-[1-(4-fluorobenzyl)-1H-pyrazol-4-yl]benzamide

3-[(2,5-Dichlorophenoxy)methyl]benzoic acid (131 mg) was suspended in tetrahydrofuran (30 mL), and oxalyl chloride (0.043 mL) and N,N-dimethylformamide (catalytic amount) were added. After stirring at room temperature for 30 min, the mixture was added dropwise to a suspension of 1-(4-fluorobenzyl)-1H-pyrazol-4-amine hydrochloride (100 mg) and triethylamine (0.07 mL) in N,N-dimethylacetamide (30 mL). After stirring at room temperature for 5 hr, ethyl acetate was added, and the mixture was washed 3 times with saturated aqueous sodium hydrogen carbonate solution. The organic layer was dried over sodium sulfate and concentrated under reduced pressure. The residue was purified by silica gel chromatography and recrystallized from ethyl acetate/hexane to give the object product (108 mg, 52%).

¹H NMR (300 MHz, DMSO-d₆) δ ppm 5.25-5.37 (m, 4H) 7.06 (dd, J=2.27, 8.33 Hz, 1H) 7.18 (t, J=8.90 Hz, 2H) 7.27-7.35 (m, 2H) 7.39 (d, J=2.27 Hz, 1H) 7.49 (d, J=8.33 Hz, 1H) 7.53-7.71 (m, 3H) 7.92 (d, J=7.95 Hz, 1H) 8.02 (s, 1H) 8.18 (s, 1H) 10.53 (s, 1H)

### Example 3

### N-[1-(4-fluorobenzyl)-1H-pyrazol-4-yl]-3-{[2-(trifluoromethyl)phenoxy]methyl}benzamide

### (1)-3-(chloromethyl)-N-[1-(4-fluorobenzyl)-1H-pyrazol-4-yl]benzamide

3-(Chloromethyl)benzoic acid (3.07 g) was suspended in tetrahydrofuran (100 mL), and oxalyl chloride (1.71 mL) and N,N-dimethylformamide (catalytic amount) were added. After stirring at room temperature for 30 min, the mixture was added dropwise to a suspension of 1-(4-fluorobenzyl)-1H-pyrazol-4-amine hydrochloride (4.0 g) and triethylamine (2.8 mL) in N,N-dimethylacetamide (100 mL). After stirring at room temperature for 5 hr, ethyl acetate was added, and the mixture was washed 3 times with saturated aqueous sodium hydrogen carbonate solution. The organic layer was dried over sodium sulfate and concentrated under reduced pressure. The residue was purified by silica gel chromatography and recrystallized from ethyl acetate/hexane to give the object product (5.5 g, 89%).

¹H NMR (300 MHz, DMSO-d₆) δ ppm 4.85 (s, 2H) 5.30 (s, 2H) 6.27 (t, J=2.08 Hz, 1H) 7.23 (d, J=8.71 Hz, 2H) 7.46 (d, J=1.51 Hz, 1H) 7.54 (t, J=7.76 Hz, 1H) 7.66 (d, J=7.57 Hz, 1H) 7.72 (d, J=8.71 Hz, 2H) 7.80 (d, J=1.89 Hz, 1H) 7.92 (d, J=7.57 Hz, 1H) 8.00 (s, 1H) 10.32 (s, 1H)

### (2) N-[1-(4-fluorobenzyl)-1H-pyrazol-4-yl]-3-{[2-(trifluoromethyl)phenoxy]methyl}benzamide

3-(Chloromethyl)-N-[1-(4-fluorobenzyl)-1H-pyrazol-4-yl]benzamide (300 mg) obtained in the above-mentioned reaction, 2-(trifluoromethyl)phenol (162 mg) and potassium carbonate (137 mg) were suspended in N,N-dimethylformamide (8 mL), and the suspension was stirred at 70° C. overnight. After cooling to room temperature, ethyl acetate was added, and the mixture was washed 3 times with saturated aqueous sodium hydrogen carbonate solution. The organic layer was dried over sodium sulfate and concentrated under reduced pressure. The residue was purified by silica gel chromatography and recrystallized from ethyl acetate/hexane to give the object product (243 mg, 60%).

¹H NMR (300 MHz, DMSO-d₆) δ ppm 5.31 (s, 2H) 5.36 (s, 2H) 7.08-7.23 (m, 3H) 7.27-7.40 (m, 3H) 7.52-7.59 (m, 1H) 7.60-7.68 (m, 4H) 7.90 (d, J=7.72 Hz, 1H) 8.02 (s, 1H) 8.18 (s, 1H) 10.50 (s, 1H)

### Example 4

### 3-[(2-cyanophenoxy)methyl]-N-[1-(4-fluorobenzyl)-1H-pyrazol-4-yl]benzamide

In the same manner as in Example 3 and using 2-cyanophenol, the object compound (49%) was obtained.

¹H NMR (300 MHz, DMSO-d₆) δ ppm 5.31 (s, 2H) 5.37 (s, 2H) 7.07-7.24 (m, 3H) 7.28-7.39 (m, 3H) 7.58 (t, J=7.63 Hz, 1H) 7.63 (s, 1H) 7.64-7.72 (m, 2H) 7.76 (dd, J=1.51, 7.72 Hz, 1H) 7.92 (d, J=7.91 Hz, 1H) 8.03 (s, 1H) 8.17 (s, 1H) 10.52 (s, 1H)

### Example 5

### 3-[(2-chlorophenoxy)methyl]-N-[1-(4-fluorobenzyl)-1H-pyrazol-4-yl]benzamide

In the same manner as in Example 3 and using 2-chlorophenol, the object compound (61%) was obtained.

¹H NMR (300 MHz, DMSO-d₆) δ ppm 5.29 (s, 2H) 5.31 (s, 2H) 6.94-7.01 (m, 1H) 7.13-7.22 (m, 2H) 7.23-7.36 (m, 4H) 7.45 (dd, J=1.41, 7.82 Hz, 1H) 7.56 (t, J=7.72 Hz, 1H) 7.63 (s, 1H) 7.64-7.70 (m, 1H) 7.91 (d, J=7.72 Hz, 1H) 8.03 (s, 1H) 8.17 (s, 1H) 10.51 (s, 1H)

### Example 6

### N-[1-(4-fluorobenzyl)-1H-pyrazol-4-yl]-3-[(2-methylphenoxy)methyl]benzamide

In the same manner as in Example 3 and using 2-methylphenol, the object compound (65%) was obtained.

¹H NMR (300 MHz, DMSO-d₆) δ ppm 2.22 (s, 3H) 5.20 (s, 2H) 5.31 (s, 2H) 6.81-6.90 (m, 1H) 7.02 (d, J=7.72 Hz, 1H) 7.10-7.22 (m, 4H) 7.27-7.36 (m, 2H) 7.54 (t, J=7.63 Hz, 1H) 7.61-7.70 (m, 2H) 7.89 (d, J=7.91 Hz, 1H) 8.03 (s, 1H) 8.17 (s, 1H) 10.50 (s, 1H)

### Example 7

### N-[1-(4-fluorobenzyl)-1H-pyrazol-4-yl]-3-[(2-isoxazol-5-ylphenoxy)methyl]benzamide

In the same manner as in Example 3 and using 2-isoxazol-5-ylphenol, the object compound (13%) was obtained.

¹H NMR (300 MHz, DMSO-d₆) δ ppm 5.31 (s, 2H) 5.39 (s, 2H) 6.76 (d, J=1.88 Hz, 1H) 7.10-7.23 (m, 3H) 7.26-7.38 (m, 3H) 7.46-7.54 (m, 1H) 7.58 (t, J=7.63 Hz, 1H) 7.63 (s, 1H) 7.71 (d, J=7.72 Hz, 1H) 7.87-7.98 (m, 2H) 8.08 (s, 1H) 8.18 (s, 1H) 8.61 (d, J=1.88 Hz, 1H) 10.52 (s, 1H)

### Example 8

### N-[1-(4-fluorobenzyl)-1H-pyrazol-4-yl]-3-[(2-fluorophenoxy)methyl]benzamide

In the same manner as in Example 3 and using 2-fluorophenol, the object compound (60%) was obtained.

¹H NMR (300 MHz, DMSO-d₆) δ ppm 5.26 (s, 2H) 5.31 (s, 2H) 6.92-7.00 (m, 1H) 7.09-7.25 (m, 4H) 7.25-7.36 (m, 3H) 7.51-7.58 (m, 1H) 7.61-7.68 (m, 2H) 7.87-7.94 (m, 1H) 8.02 (s, 1H) 8.16 (s, 1H) 10.51 (s, 1H)

### Example 9

### N-[1-(4-fluorobenzyl)-1H-pyrazol-4-yl]-3-{[2-(trifluoromethoxy)phenoxy]methyl}benzamide

In the same manner as in Example 3 and using 2-(trifluoromethoxy)phenol, the object compound (67%) was obtained.

¹H NMR (300 MHz, DMSO-d₆) δ ppm 5.30 (s, 2H) 5.31 (s, 2H) 7.00-7.08 (m, 1H) 7.12-7.23 (m, 2H) 7.27-7.41 (m, 5H) 7.51-7.59 (m, 1H) 7.61-7.66 (m, 2H) 7.90 (d, J=7.54 Hz, 1H) 8.01 (s, 1H) 8.18 (s, 1H) 10.50 (s, 1H)

### Example 10

### N-[1-(4-fluorobenzyl)-1H-pyrazol-4-yl]-3-{[2-(1-methyl-1H-pyrazol-5-yl)phenoxy]methyl}benzamide

In the same manner as in Example 3 and using 2-(1-methyl-1H-pyrazol-5-yl)phenol, the object compound (68%) was obtained.

¹H NMR (300 MHz, DMSO-d₆) δ ppm 3.65 (s, 3H) 5.24 (s, 2H) 5.32 (s, 2H) 6.29 (d, J=1.70 Hz, 1H) 7.04-7.11 (m, 1H) 7.13-7.23 (m, 2H) 7.24-7.37 (m, 4H) 7.41-7.49 (m, 2H) 7.49-7.55 (m, 2H) 7.65 (s, 1H) 7.85-7.92 (m, 1H) 7.94 (s, 1H) 8.20 (s, 1H) 10.49 (s, 1H)

### Example 11

### 3-[(3-chlorophenoxy)methyl]-N-[1-(4-fluorobenzyl)-1H-pyrazol-4-yl]benzamide

In the same manner as in Example 3 and using 3-chlorophenol, the object compound (75%) was obtained.

¹H NMR (300 MHz, DMSO-d₆) δ ppm 5.21 (s, 2H) 5.31 (s, 2H) 7.01 (d, J=2.07 Hz, 1H) 7.03 (d, J=2.07 Hz, 1H) 7.12-7.22 (m, 3H) 7.27-7.37 (m, 3H) 7.50-7.59 (m, 1H) 7.60-7.67 (m, 2H) 7.90 (d, J=7.91 Hz, 1H) 8.01 (s, 1H) 8.16 (s, 1H) 10.50 (s, 1H)

### Example 12

### 3-[(4-chlorophenoxy)methyl]-N-[1-(4-fluorobenzyl)-1H-pyrazol-4-yl]benzamide

In the same manner as in Example 3 and using 4-chlorophenol, the object compound (75%) was obtained.

¹H NMR (300 MHz, DMSO-d₆) δ ppm 5.18 (s, 2H) 5.31 (s, 2H) 7.03-7.10 (m, 2H) 7.13-7.23 (m, 2H) 7.27-7.38 (m, 4H) 7.54 (t, J=7.63 Hz, 1H) 7.60-7.67 (m, 2H) 7.87-7.94 (m, 1H) 8.01 (s, 1H) 8.16 (s, 1H) 10.50 (s, 1H)

### Example 13

### 5-butoxy-1-(2,4-dichlorobenzyl)-N-(4,6-dimethylpyridin-2-yl)-1H-pyrazole-3-carboxamide

### (1) methyl 5-butoxy-1H-pyrazole-3-carboxylate

A solution of methyl 5-hydroxy-1H-pyrazole-3-carboxylate (60.8 g), butyl iodide (82.7 g) and potassium carbonate (59.2 g) in N,N-dimethylformamide (500 mL) was stirred at 50° C. overnight. After cooling, water was added to the reaction mixture, and the mixture was extracted with ethyl acetate. The organic layer was washed with saturated aqueous solution and dried over sodium sulfate, and the solvent was evaporated under reduced pressure. The residue was purified by silica gel column (25% ethyl acetate/hexane to 30% ethyl acetate/hexane) to give the object product (59.8 g, 71%) as a white solid.

¹H NMR (300 MHz, CDCl₃) δ ppm 0.97 (t, J=7.4 Hz, 3H) 1.41-1.80 (m, 2H) 3.91 (s, 3H) 4.17 (t, J=6.5 Hz, 2H) 6.21 (s, 1H) 10.29 (brs, 1H)

### (2) methyl [5-butoxy-1-(2,4-dichlorobenzyl)-1H-pyrazol-3-yl](oxo)acetate

By reaction in the same manner as in Example 13(1) and using methyl 5-butoxy-1H-pyrazole-3-carboxylate (38 g) obtained in the above-mentioned reaction, 2,4-dichloro-1-(chloromethyl)benzene (39.5 g) and potassium carbonate (26.5 g), the object compound (15 g, 22%) was obtained as a white solid.

¹H NMR (300 MHz, CDCl₃) δ ppm 0.84-0.97 (m, 3H) 1.35 (dd, J=7.44, 14.98 Hz, 2H) 1.64-1.78 (m, 2H) 3.91 (s, 3H) 4.05 (t, J=6.50 Hz, 2H) 5.31 (s, 2H) 6.12 (s, 1H) 6.73 (d, J=8.29 Hz, 1H) 7.15 (dd, J=2.07, 8.29 Hz, 1H) 7.39 (d, J=2.07 Hz, 1H)

### (3) 5-butoxy-1-(2,4-dichlorobenzyl)-1H-pyrazole-3-carboxylic acid

A mixed solution of methyl [5-butoxy-1-(2,4-dichlorobenzyl)-1H-pyrazol-3-yl](oxo)acetate (7.4 g) obtained in the above-mentioned reaction and 1N aqueous sodium hydroxide solution (30 mL) in methanol and tetrahydrofuran (1:1, 30 mL) was stirred at 50° C. for 2 hr. After cooling, 1N hydrochloric acid (30 mL) was added, and the obtained precipitate was collected by filtration and dried to give the object product (6.6 g, 93%) as a white solid.

¹H NMR (300 MHz, CDCl₃) δ ppm 0.92 (t, J=7.3 Hz, 3H) 1.30-1.43 (m, 2H) 1.67-1.77 (m, 2H) 4.07 (t, J=6.4 Hz, 2H) 5.34 (s, 2H) 6.15 (s, 1H) 6.81 (d, J=8.3 Hz, 1H) 7.17 (dd, J=2.1, 8.3 Hz, 1H) 7.40 (d, J=2.1 Hz, 1H)

### (4) 5-butoxy-1-(2,4-dichlorobenzyl)-N-(4,6-dimethylpyridin-2-yl)-1H-pyrazole-3-carboxamide

A solution of 5-butoxy-1-(2,4-dichlorobenzyl)-1H-pyrazole-3-carboxylic acid (300 mg) obtained in the above-mentioned reaction, 4,6-dimethylpyridin-2-amine (107 mg), 1-ethyl-3-(3-dimethylaminopropyl)carbodiimide hydrochloride (250 mg), hydroxybenzotriazole hydrate (200 mg), N-methylimidazole (0.1 mL) and triethylamine (0.2 mL) in tetrahydrofuran (6 mL) was stirred at room temperature overnight. Water was added to the reaction mixture, and the mixture was extracted with ethyl acetate. The organic layer was washed with saturated aqueous solution and dried over sodium sulfate, and the solvent was evaporated under reduced pressure. The residue was purified by silica gel column (30% ethyl acetate/hexane to 40% ethyl acetate/hexane) to give the object product (140 mg, 36%) as a pale-yellow solid.

¹H NMR (300 MHz, CDCl₃) δ ppm 0.92 (t, J=6.9 Hz, 3H) 1.34-1.42 (m, 2H) 1.70-1.75 (m, 2H) 2.33 (s, 3H) 2.41 (s, 3H) 4.08 (t, J=6.3 Hz, 2H) 5.25 (s, 2H) 6.16 (s, 1H) 6.72 (s, 1H) 6.79 (d, J=8.1 Hz, 1H) 7.16 (dd, J=2.4, 8.7 Hz, 1H) 7.41 (d, J=2.1 Hz, 1H) 7.97 (s, 1H) 9.13 (s, 1H)

### Example 14

### 4-[(4-chlorophenyl)sulfonyl]-N-(4,6-dimethylpyridin-2-yl)-3-methylthiophene-2-carboxamide

By reaction in the same manner as in Example 13 and using 4-[(4-chlorophenyl)sulfonyl]-3-methylthiophene-2-carboxylic acid (300 mg) and 4,6-dimethylpyridin-2-amine (160 mg), the object compound (55 mg, 14%) was obtained as a white solid.

¹H NMR (300 MHz, CDCl₃) δ ppm 2.34 (s, 3H) 2.39 (s, 3H) 2.56 (s, 3H) 6.77 (s, 1H) 7.51 (d, J=8.7 Hz, 2H) 7.82 (brs, 1H) 7.84 (d, J=8.7 Hz, 2H) 8.11 (brs, 1H) 8.35 (s, 1H)

### Example 15

### 3-[(2-chlorophenoxy)methyl]-N-[4-(1H-pyrazol-1-ylmethyl)phenyl]piperidine-1-carboxamide

### (1) N-tert-butoxycarbonyl-3-(hydroxymethyl)piperidine

To a solution of piperidin-3-ylmethanol (5 g) in methanol (200 mL) was added dropwise di-tert-butyl bicarbonate (13.1 mL), and the mixture was stirred at room temperature overnight. The solvent was evaporated under reduced pressure, and ethyl acetate was added. The mixture was washed 3 times with saturated aqueous sodium hydrogen carbonate solution, and the organic layer was dried over sodium sulfate. The solvent was evaporated under reduced pressure to give a crude object product (6.49 g). The obtained crude product was used for the next reaction without further purification.

### (2) N-tert-butoxycarbonyl-3-[(2-chlorophenoxy)methyl]piperidine

To a solution (40 mL) of N-tert-butoxycarbonyl-3-(hydroxymethyl)piperidine (1 g) obtained in the above-mentioned reaction, 2-chlorophenol (0.477 mL) and triphenylphosphine (1.31 g) in tetrahydrofuran was added dropwise diethylazodicarboxylic acid (40% toluene solution, 2.18 mL) with stirring. After stirring at 70° C. overnight, the mixture was cooled to room temperature, and ethyl acetate was added. The mixture was washed 3 times with saturated aqueous sodium hydrogen carbonate solution, the organic layer was dried over sodium sulfate, and the solvent was evaporated under reduced pressure. The residue was purified by silica gel column chromatography to give the object compound (1.3 g, 87%).

¹H NMR (300 MHz, DMSO-d₆) δ ppm 4.85 (s, 2H) 5.30 (s, 2H) 6.27 (t, J=2.08 Hz, 1H) 7.23 (d, J=8.71 Hz, 2H) 7.46 (d, J=1.51 Hz, 1H) 7.54 (t, J=7.76 Hz, 1H) 7.66 (d, J=7.57 Hz, 1H) 7.72 (d, J=8.71 Hz, 2H) 7.80 (d, J=1.89 Hz, 1H) 7.92 (d, J=7.57 Hz, 1H) 8.00 (s, 1H) 10.32 (s, 1H)

### (3) 3-[(2-chlorophenoxy)methyl]piperidine hydrochloride

To a solution of N-tert-butoxycarbonyl-3-[(2-chlorophenoxy)methyl]piperidine (1.3 g) obtained in the above-mentioned reaction in ethyl acetate (30 mL) was added dropwise hydrochloric acid (4N ethyl acetate solution, 5 mL) with stirring, and the mixture was stirred at room temperature for 4 hr. The precipitate was collected by filtration and dried under reduced pressure to give the object product as a crude product (1.24 g). The obtained crude product was used for the next reaction without further purification.

### (4) phenyl[4-(1H-pyrazol-1-ylmethyl)phenyl]carbamate

To a suspension of 4-(1H-pyrazol-1-ylmethyl)aniline (866 mg) and triethylamine (0.77 mL) in tetrahydrofuran (50 mL) was added dropwise phenyl chloroformate (0.7 mL) with stirring under ice-cooling. After stirring at room temperature overnight, ethyl acetate was added, and the mixture was washed 3 times with saturated aqueous sodium hydrogen carbonate solution. The organic layer was dried over sodium sulfate, and the solvent was evaporated under reduced pressure. Ethyl acetate/hexane was added to the residue, and the resulting precipitate was collected by filtration to give the object product as a crude product (1.34 g). The obtained crude product was used for the next reaction without further purification.

### (5) 3-[(2-chlorophenoxy)methyl]-N-[4-(1H-pyrazol-1-ylmethyl)phenyl]piperidine-1-carboxamide

A solution of 3-[(2-chlorophenoxy)methyl]piperidine hydrochloride (210 mg), phenyl [4-(1H-pyrazol-1-ylmethyl)phenyl]carbamate (235 mg) and triethylamine (0.28 mL) in N,N-dimethylformamide (10 mL) was stirred at 60° C. overnight. After cooling to room temperature, ethyl acetate was added, and the mixture was washed 3 times with saturated aqueous sodium hydrogen carbonate solution. The organic layer was dried over sodium sulfate and the solvent was evaporated under reduced pressure. The residue was purified by silica gel column chromatography to give the object product (170 mg, 50%).

¹H NMR (300 MHz, DMSO-d₆) δ ppm 1.28-1.56 (m, 2H) 1.63-1.76 (m, 1H) 1.85-1.99 (m, 2H) 2.76 (dd, J=10.17, 13.00 Hz, 1H) 2.82-2.94 (m, 1H) 3.90-4.01 (m, 3H) 4.12 (m, 1H) 5.22 (s, 2H) 6.24 (t, J=1.98 Hz, 1H) 6.89-6.99 (m, 1H) 7.07-7.20 (m, 3H) 7.23-7.33 (m, 1H) 7.35-7.47 (m, 4H) 7.75 (d, J=1.70 Hz, 1H) 8.49 (s, 1H)

### Example 16

### 3-[(2-chlorophenoxy)methyl]-N-[1-(4-fluorobenzyl)-1H-pyrazol-4-yl]piperidine-1-carboxamide

### (1) phenyl[1-(4-fluorobenzyl)-1H-pyrazol-4-yl]carbamate

Using 1-(4-fluorobenzyl)-1H-pyrazol-4-amine hydrochloride and in the same manner as in the synthesis of phenyl [4-(1H-pyrazol-1-ylmethyl)phenyl]carbamate shown in Example 15, the object product was obtained as a crude product. The obtained crude product was used for the next reaction without further purification.

### (2) 3-[(2-chlorophenoxy)methyl]-N-[1-(4-fluorobenzyl)-1H-pyrazol-4-yl]piperidine-1-carboxamide

In the same manner as in Example 15 and using phenyl [1-(4-fluorobenzyl)-1H-pyrazol-4-yl]carbamate obtained in the above-mentioned reaction and 3-[(2-chlorophenoxy)methyl]piperidine hydrochloride obtained in Example 15(3), the object compound (46%) was obtained.

¹H NMR (300 MHz, DMSO-d₆) δ ppm 1.26-1.52 (m, 2H) 1.63-1.75 (m, 1H) 1.81-1.98 (m, 2H) 2.71 (dd, J=10.22, 12.87 Hz, 1H) 2.78-2.90 (m, 1H) 3.85-3.97 (m, 3H) 4.09-4.19 (m, 1H) 5.21 (s, 2H) 6.91-6.99 (m, 1H) 7.10-7.20 (m, 3H) 7.22-7.32 (m, 3H) 7.38 (s, 1H) 7.42 (dd, J=1.70, 7.76 Hz, 1H) 7.77 (s, 1H) 8.49 (s, 1H)

### Example 17

### 3-[(2-chlorophenoxy)methyl]-N-(4,6-dimethylpyridin-2-yl)piperidine-1-carboxamide

### (1) phenyl(4,6-dimethylpyridin-2-yl)carbamate

Using 4,6-dimethylpyridin-2-amine and in the same manner as in the synthesis of phenyl [4-(1H-pyrazol-1-ylmethyl)phenyl]carbamate shown in Example 15, the object product was obtained as a crude product. The obtained crude product was used for the next reaction without further purification.

### (2) 3-[(2-chlorophenoxy)methyl]-N-(4,6-dimethylpyridin-2-yl)piperidine-1-carboxamide

In the same manner as in Example 15 and using phenyl (4,6-dimethylpyridin-2-yl)carbamate obtained in the above-mentioned reaction and 3-[(2-chlorophenoxy)methyl]piperidine hydrochloride obtained in Example 15(3), the object compound (74%) was obtained.

¹H NMR (300 MHz, DMSO-d₆) δ ppm 1.31-1.52 (m, 2H) 1.63-1.75 (m, 1H) 1.82-2.02 (m, 2H) 2.21 (s, 3H) 2.31 (s, 3H) 2.72-2.93 (m, 2H) 3.91-4.06 (m, 3H) 4.14-4.24 (m, 1H) 6.65 (s, 1H) 6.91-6.98 (m, 1H) 7.15 (dd, J=1.32, 8.29 Hz, 1H) 7.25-7.33 (m, 1H) 7.38-7.45 (m, 2H) 8.89 (s, 1H)

### Example 18

### 2-[(2-chlorophenoxy)methyl]-N-[4-(1H-pyrazol-1-ylmethyl)phenyl]morpholine-4-carboxamide

### (1) N-tert-butoxycarbonyl-2-[(2-chlorophenoxy)methyl]morpholine

Using N-tert-butoxycarbonyl-2-(hydroxymethyl)morpholine, and in the same manner as in the synthesis of N-tert-butoxycarbonyl-3-[(2-chlorophenoxy)methyl]piperidine shown in Example 15, the object product (86%) was obtained.

¹H NMR (300 MHz, DMSO-d₆) δ ppm 1.41 (s, 9H) 2.74-3.02 (m, 2H) 3.39-3.52 (m, 1H) 3.66-3.79 (m, 2H) 3.82-3.90 (m, 1H) 3.95-4.10 (m, 2H) 4.10-4.18 (m, 1H) 6.91-7.01 (m, 1H) 7.13-7.20 (m, 1H) 7.24-7.35 (m, 1H) 7.42 (dd, J=1.70, 7.76 Hz, 1H)

### (2) 2-[(2-chlorophenoxy)methyl]morpholine hydrochloride

Using N-tert-butoxycarbonyl-2-[(2-chlorophenoxy)methyl]morpholine obtained in the above-mentioned reaction and in the same manner as in the synthesis of 3-[(2-chlorophenoxy)methyl]piperidine hydrochloride shown in Example 15, the object product was obtained as a crude product. The obtained crude product was used for the next reaction without further purification.

### (3) 2-[(2-chlorophenoxy)methyl]-N-[4-(1H-pyrazol-1-ylmethyl)phenyl]morpholine-4-carboxamide

In the same manner as in Example 15 and using 2-[(2-chlorophenoxy)methyl]morpholine hydrochloride obtained in the above-mentioned reaction and phenyl [4-(1H-pyrazol-1-ylmethyl)phenyl]carbamate obtained in Example 15(4), the object compound (37%) was obtained.

¹H NMR (300 MHz, DMSO-d₆) δ ppm 2.81-3.04 (m, 2H) 3.47-3.59 (m, 1H) 3.71-3.83 (m, 1H) 3.88-3.98 (m, 2H) 4.10-4.19 (m, 3H) 5.23 (s, 2H) 6.25 (t, J=2.07 Hz, 1H) 6.92-7.01 (m, 1H) 7.12 (d, J=8.48 Hz, 2H) 7.16-7.21 (m, 1H) 7.27-7.34 (m, 1H) 7.38-7.45 (m, 4H) 7.74-7.78 (m, 1H) 8.61 (s, 1H)

### Example 19

### 2-[(2-chlorophenoxy)methyl]-N-[1-(4-fluorobenzyl)-1H-pyrazol-4-yl]morpholine-4-carboxamide

In the same manner as in Example 15 and using 2-[(2-chlorophenoxy)methyl]morpholine hydrochloride obtained in Example 18(2) and phenyl [1-(4-fluorobenzyl)-1H-pyrazol-4-yl]carbamate obtained in Example 16(1), the object compound (51%) was obtained.

¹H NMR (300 MHz, DMSO-d₆) δ ppm 2.77-2.98 (m, 2H) 3.43-3.57 (m, 1H) 3.70-3.80 (m, 1H) 3.81-3.95 (m, 2H) 4.03-4.14 (m, 3H) 5.23 (s, 2H) 6.93-7.01 (m, 1H) 7.12-7.21 (m, 3H) 7.22-7.34 (m, 3H) 7.39 (s, 1H) 7.43 (dd, J=1.51, 7.95 Hz, 1H) 7.79 (s, 1H) 8.62 (s, 1H)

### Example 20

### 2-[(2-chlorophenoxy)methyl]-N-(4,6-dimethylpyridin-2-yl)morpholine-4-carboxamide

In the same manner as in Example 15 and using 2-[(2-chlorophenoxy)methyl]morpholine hydrochloride obtained in Example 18(2) and phenyl (4,6-dimethylpyridin-2-yl)carbamate obtained in Example 17(1), the object compound (41%) was obtained.

¹H NMR (300 MHz, DMSO-d₆) δ ppm 2.23 (s, 3H) 2.33 (s, 3H) 2.80-3.00 (m, 2H) 3.44-3.56 (m, 1H) 3.71-3.82 (m, 1H) 3.86-3.93 (m, 1H) 3.95-4.03 (m, 1H) 4.13 (d, J=4.71 Hz, 2H) 4.19 (d, J=13.00 Hz, 1H) 6.68 (s, 1H) 6.93-7.01 (m, 1H) 7.16-7.22 (m, 1H) 7.27-7.34 (m, 1H) 7.41-7.47 (m, 2H) 9.05 (s, 1H)

### Example 21

### 3-[(2-chlorophenoxy)methyl]-N-[1-(4-fluorobenzyl)-1H-pyrazol-4-yl]pyrrolidine-1-carboxamide

### (1) N-tert-butoxycarbonyl-3-[(2-chlorophenoxy)methyl]pyrrolidine

Using N-tert-butoxycarbonyl-3-(hydroxymethyl)pyrrolidine and in the same manner as in the synthesis of N-tert-butoxycarbonyl-3-[(2-chlorophenoxy)methyl]piperidine shown in Example 15, the object product (78%) was obtained.

¹H NMR (300 MHz, DMSO-d₆) δ ppm 1.40 (s, 9H) 1.65-1.83 (m, 1H) 1.92-2.09 (m, 1H) 2.54-2.74 (m, 1H) 3.07-3.19 (m, 1H) 3.19-3.31 (m, 1H) 3.34-3.42 (m, 1H) 3.46 (m, 1H) 3.97-4.10 (m, 2H) 6.91-7.00 (m, 1H) 7.15 (dd, J=1.41, 8.38 Hz, 1H) 7.25-7.33 (m, 1H) 7.42 (dd, J=1.70, 7.91 Hz, 1H)

### (2) 3-[(2-chlorophenoxy)methyl]pyrrolidine hydrochloride

Using N-tert-butoxycarbonyl-3-[(2-chlorophenoxy)methyl]pyrrolidine obtained in the above-mentioned reaction and in the same manner as in the synthesis of 3-[(2-chlorophenoxy)methyl]piperidine hydrochloride shown in Example 15, the object product was obtained as a crude product. The obtained crude product was used for the next reaction without further purification.

### (3) 3-[(2-chlorophenoxy)methyl]-N-[1-(4-fluorobenzyl)-1H-pyrazol-4-yl]pyrrolidine-1-carboxamide

In the same manner as in Example 15 and using 3-[(2-chlorophenoxy)methyl]pyrrolidine hydrochloride obtained in the above-mentioned reaction and phenyl [1-(4-fluorobenzyl)-1H-pyrazol-4-yl]carbamate obtained in Example 16(1), the object compound (52%) was obtained.

¹H NMR (300 MHz, CDCl₃) δ ppm 1.73-1.92 (m, 1H) 1.99-2.16 (m, 1H) 2.63-2.80 (m, 1H) 3.25-3.46 (m, 2H) 3.48-3.59 (m, 1H) 3.59-3.69 (m, 1H) 3.83-3.98 (m, 2H) 5.09 (s, 2H) 6.82-6.98 (m, 5H) 7.08-7.22 (m, 3H) 7.32 (dd, J=1.51, 7.72 Hz, 1H) 7.37 (s, 1H) 7.72 (s, 1H)

### Example 22

### 3-[(2-chlorophenoxy)methyl]-N-(4,6-dimethylpyridin-2-yl)pyrrolidine-1-carboxamide

In the same manner as in Example 15 and using 3-[(2-chlorophenoxy)methyl]pyrrolidine hydrochloride obtained in Example 21(2) and phenyl (4,6-dimethylpyridin-2-yl)carbamate obtained in Example 17(1), the object compound (50%) was obtained.

¹H NMR (300 MHz, CDCl₃) δ ppm 1.88-2.07 (m, 1H) 2.12-2.25 (m, 1H) 2.27 (s, 3H) 2.36 (s, 3H) 2.71-2.89 (m, 1H) 3.43 (dd, J=6.78, 10.17 Hz, 1H) 3.48-3.58 (m, 1H) 3.61-3.72 (m, 1H) 3.77 (dd, J=7.35, 10.17 Hz, 1H) 4.00 (dd, J=0.94, 6.59 Hz, 2H) 6.62 (s, 1H) 6.84-6.94 (m, 2H) 7.07 (brs, 1H) 7.16-7.24 (m, 1H) 7.32-7.38 (m, 1H) 7.76 (s, 1H)

### Example 23

### 2-[(2,5-dichlorophenoxy)methyl]-N-[1-(4-fluorobenzyl)-1H-pyrazol-4-yl]morpholine-4-carboxamide hydrochloride

### (1) N-[1-(4-fluorobenzyl)-1H-pyrazol-4-yl]-2-(hydroxymethyl)morpholine-4-carboxamide

A suspension of phenyl [1-(4-fluorobenzyl)-1H-pyrazol-4-yl]carbamate (6.23 g) obtained in Example 16(1) and morpholin-2-ylmethanol (2.6 g) in 2-propanol was stirred under reflux overnight. The solvent was evaporated under reduced pressure, and the residue was purified by silica gel column chromatography to give the object product as a crude product. The obtained crude product was used for the next reaction without further purification.

### (2) 2-[(2,5-dichlorophenoxy)methyl]-N-[1-(4-fluorobenzyl)-1H-pyrazol-4-yl]morpholine-4-carboxamide hydrochloride

Using N-[1-(4-fluorobenzyl)-1H-pyrazol-4-yl]-2-(hydroxymethyl)morpholine-4-carboxamide obtained in the above-mentioned reaction and 2,5-dichlorophenol and in the same manner as in the synthesis of N-tert-butoxycarbonyl-3-[(2-chlorophenoxy)methyl]piperidine shown in Example 15, the object product was obtained. The product was dissolved again in a dioxane solution, and hydrochloric acid (4N ethyl acetate solution, 5 mL) was added dropwise with stirring. The resulting precipitate was collected by filtration to give the object product (18%) as a hydrochloride.

¹H NMR (300 MHz, DMSO-d₆) δ ppm 2.75-2.98 (m, 2H) 3.44-3.58 (m, 1H) 3.74 (dd, J=2.27, 10.22 Hz, 1H) 3.81-3.95 (m, 2H) 4.05-4.18 (m, 3H) 5.23 (s, 2H) 7.01-7.07 (m, 1H) 7.16 (t, J=8.90 Hz, 2H) 7.22-7.32 (m, 3H) 7.37-7.48 (m, 2H) 7.79 (s, 1H) 8.65 (s, 1H)

### Example 24

### 3-[2-(2-chlorophenoxy)ethyl]-N-(4,6-dimethylpyridin-2-yl)piperidine-1-carboxamide

### (1) N-tert-butoxycarbonyl-3-[2-(2-chlorophenoxy)ethyl]piperidine

Using N-tert-butoxycarbonyl-3-(2-hydroxyethyl)piperidine and in the same manner as in the synthesis of N-tert-butoxycarbonyl-3-[(2-chlorophenoxy)methyl]piperidine shown in Example 15, the object product (86%) was obtained.

¹H NMR (300 MHz, DMSO-d₆) δ ppm 1.08-1.30 (m, 2H) 1.35 (s, 9H) 1.51-1.73 (m, 4H) 1.75-1.88 (m, 1H) 2.54-2.72 (m, 1H) 2.75-2.89 (m, 1H) 3.72 (d, J=13.25 Hz, 1H) 3.77-3.91 (m, 1H) 4.05-4.15 (m, 2H) 6.94 (t, J=7.00 Hz, 1H) 7.14 (d, J=8.33 Hz, 1H) 7.22-7.34 (m, 1H) 7.40 (d, J=7.95 Hz, 1H)

### (2) 3-[2-(2-chlorophenoxy)ethyl]piperidine hydrochloride

Using N-tert-butoxycarbonyl-3-[2-(2-chlorophenoxy)ethyl]piperidine obtained in the above-mentioned reaction and in the same manner as in the synthesis of 3-[(2-chlorophenoxy)methyl]piperidine hydrochloride shown in Example 15, the object product was obtained as a crude product. The obtained crude product was used for the next reaction without further purification.

### (3) 3-[2-(2-chlorophenoxy)ethyl]-N-(4,6-dimethylpyridin-2-yl)piperidine-1-carboxamide

In the same manner as in Example 15 and using 3-[2-(2-chlorophenoxy)ethyl]piperidine hydrochloride obtained in the above-mentioned reaction and phenyl (4,6-dimethylpyridin-2-yl)carbamate obtained in Example 17(1), the object compound (53%) was obtained.

¹H NMR (300 MHz, CDCl₃) δ ppm 1.11-1.33 (m, 1H) 1.38-1.55 (m, 1H) 1.58-1.92 (m, 5H) 2.71-2.97 (m, 2H) 3.83-3.94 (m, 1H) 3.95-4.14 (m, 3H) 5.11 (s, 2H) 6.82-7.05 (m, 5H) 7.08-7.24 (m, 3H) 7.29-7.39 (m, 2H) 7.71 (s, 1H)

### Example 25

### 2-[(3-chlorophenoxy)methyl]-N-[1-(4-fluorobenzyl)-1H-pyrazol-4-yl]morpholine-4-carboxamide hydrochloride

Using N-[1-(4-fluorobenzyl)-1H-pyrazol-4-yl]-2-(hydroxymethyl)morpholine-4-carboxamide obtained in Example 23(1) and 3-chlorophenol and in the same manner as in Example 23, the object product (21%) was obtained as a hydrochloride.

¹H NMR (300 MHz, DMSO-d₆) δ ppm 2.76 (dd, J=10.60, 12.87 Hz, 1H) 2.85-2.97 (m, 1H) 3.42-3.55 (m, 1H) 3.65-3.77 (m, 1H) 3.82-3.93 (m, 2H) 3.99-4.08 (m, 3H) 5.23 (s, 2H) 6.93-7.03 (m, 2H) 7.06 (t, J=2.08 Hz, 1H) 7.11-7.20 (m, 2H) 7.22-7.35 (m, 3H) 7.41 (s, 1H) 7.79 (s, 1H) 8.65 (s, 1H)

### Example 26

### 4-[(2,5-dichlorophenoxy)methyl]-N-(4,6-dimethylpyridin-2-yl)piperidine-1-carboxamide

### (1) N-tert-butoxycarbonyl-4-[(2,5-dichlorophenoxy)methyl]piperidine

Using N-tert-butoxycarbonyl-4-(hydroxymethyl)piperidine and in the same manner as in the synthesis of N-tert-butoxycarbonyl-3-[(2-chlorophenoxy)methyl]piperidine shown in Example 15, the object product was obtained as a crude product. The obtained crude product was used for the next reaction without further purification.

### (2) 4-[(2,5-dichlorophenoxy)methyl]piperidine

Using N-tert-butoxycarbonyl-4-[(2,5-dichlorophenoxy)methyl]piperidine obtained in the above-mentioned reaction and in the same manner as in the synthesis of 3-[(2-chlorophenoxy)methyl]piperidine hydrochloride shown in Example 15, the object product was obtained as a crude product. The obtained crude product was used for the next reaction without further purification.

### (3) 4-[(2,5-dichlorophenoxy)methyl]-N-(4,6-dimethylpyridin-2-yl)piperidine-1-carboxamide

In the same manner as in Example 15 and using 4-[(2,5-dichlorophenoxy)methyl]piperidine obtained in the above-mentioned reaction, phenyl (4,6-dimethylpyridin-2-yl)carbamate obtained in Example 17(1), the object compound (67%) was obtained.

¹H NMR (300 MHz, DMSO-d₆) δ ppm 1.18-1.35 (m, 2H) 1.72-1.86 (m, 2H) 1.92-2.08 (m, 1H) 2.22 (s, 3H) 2.33 (s, 3H) 2.83 (t, J=11.77 Hz, 2H) 3.97 (d, J=6.40 Hz, 2H) 4.20 (d, J=13.19 Hz, 2H) 6.65 (s, 1H) 7.01 (dd, J=2.26, 8.48 Hz, 1H) 7.24 (d, J=2.26 Hz, 1H) 7.41-7.48 (m, 2H) 8.92 (s, 1H)

### Example 27

### 2-[(2-chloro-4-fluorophenoxy)methyl]-N-[1-(4-fluorobenzyl)-1H-pyrazol-4-yl]morpholine-4-carboxamide hydrochloride

Using N-[1-(4-fluorobenzyl)-1H-pyrazol-4-yl]-2-(hydroxymethyl)morpholine-4-carboxamide obtained in Example 23(1) and 2-chloro-4-fluorophenol and in the same manner as in Example 23, the object product (31%) was obtained as a hydrochloride.

¹H NMR (300 MHz, DMSO-d₆) δ ppm 2.74-3.00 (m, 2H) 3.44-3.55 (m, 1H) 3.65-3.79 (m, 1H) 3.81-3.97 (m, 2H) 4.02-4.15 (m, 3H) 5.23 (s, 2H) 7.08-7.30 (m, 6H) 7.37-7.48 (m, 2H) 7.80 (s, 1H) 8.67 (s, 1H)

### Example 28

### 4-[(2,5-dichlorophenoxy)methyl]-N-[4-(1H-pyrazol-1-ylmethyl)phenyl]piperidine-1-carboxamide

In the same manner as in Example 15 and using 4-[(2,5-dichlorophenoxy)methyl]piperidine obtained in Example 26(2) and phenyl [4-(1H-pyrazol-1-ylmethyl)phenyl]carbamate obtained in Example 15(4), the object compound (70%) was obtained.

¹H NMR (300 MHz, DMSO-d₆) δ ppm 1.16-1.36 (m, 2H) 1.73-1.85 (m, 2H) 1.91-2.09 (m, 1H) 2.82 (t, J=11.59 Hz, 2H) 3.99 (d, J=6.22 Hz, 2H) 4.15 (d, J=13.37 Hz, 2H) 5.22 (s, 2H) 6.24 (t, J=2.07 Hz, 1H) 7.02 (dd, J=2.45, 8.48 Hz, 1H) 7.10 (d, J=8.67 Hz, 2H) 7.26 (d, J=2.45 Hz, 1H) 7.37-7.48 (m, 4H) 7.76 (dd, J=0.75, 2.26 Hz, 1H) 8.50 (s, 1H)

### Examples 29-54

To a solution of N-[1-(4-fluorobenzyl)-1H-pyrazol-4-yl]-2-(hydroxymethyl)morpholine-4-carboxamide obtained in Example 23(1) in tetrahydrofuran were added a triphenylphosphine polystyrene resin and corresponding phenol, and then di-tert-butyl azodicarboxylate was added. The reaction mixture was stirred at 50° C. overnight. The reaction solution was filtered, and the solvent was evaporated. The residue was dissolved in a solution (1 mL) of dimethyl sulfoxide/methanol=1/1 and purified by preparative HPLC to give the object compound at a purity of not less than 80% (LCMS analysis).

### Example 29

### 2-[(2,3-dichlorophenoxy)methyl]-N-[1-(4-fluorobenzyl)-1H-pyrazol-4-yl]morpholine-4-carboxamide trifluoroacetate

ESI(pos) 479 [M+H]⁺

### Example 30

### 2-[(3,5-dichlorophenoxy)methyl]-N-[1-(4-fluorobenzyl)-1H-pyrazol-4-yl]morpholine-4-carboxamide trifluoroacetate

ESI(pos) 479 [M+H]⁺

### Example 31

### N-[1-(4-fluorobenzyl)-1H-pyrazol-4-yl]-2-[(3-fluorophenoxy)methyl]morpholine-4-carboxamide trifluoroacetate

ESI(pos) 429 [M+H]⁺

### Example 32

### N-[1-(4-fluorobenzyl)-1H-pyrazol-4-yl]-2-[(2-fluorophenoxy)methyl]morpholine-4-carboxamide trifluoroacetate

ESI(pos) 429 [M+H]⁺

### Example 33

### 2-{[(2-chloropyridin-3-yl)oxy]methyl}-N-[1-(4-fluorobenzyl)-1H-pyrazol-4-yl]morpholine-4-carboxamide trifluoroacetate

ESI(pos) 446 [M+H]⁺

### Example 34

### N-[1-(4-fluorobenzyl)-1H-pyrazol-4-yl]-2-[(pyridin-3-yloxy)methyl]morpholine-4-carboxamide trifluoroacetate

ESI(pos) 412 [M+H]⁺

### Example 35

### N-[1-(4-fluorobenzyl)-1H-pyrazol-4-yl]-2-[(pyridin-2-yloxy)methyl]morpholine-4-carboxamide trifluoroacetate

ESI(pos) 412 [M+H]⁺

### Example 36

### N-[1-(4-fluorobenzyl)-1H-pyrazol-4-yl]-2-{[(4-methylpyridin-2-yl)oxy]methyl}morpholine-4-carboxamide trifluoroacetate

ESI(pos) 426 [M+H]⁺

### Example 37

### 2-{[(6-chloropyridin-2-yl)oxy]methyl}-N-[1-(4-fluorobenzyl)-1H-pyrazol-4-yl]morpholine-4-carboxamide trifluoroacetate

ESI(pos) 446 [M+H]⁺

### Example 38

### N-[1-(4-fluorobenzyl)-1H-pyrazol-4-yl]-2-{[(2-methylpyrimidin-4-yl)oxy]methyl}morpholine-4-carboxamide trifluoroacetate

ESI(pos) 427 [M+H]⁺

### Example 39

### N-[1-(4-fluorobenzyl)-1H-pyrazol-4-yl]-2-{[2-(trifluoromethyl)phenoxy]methyl}morpholine-4-carboxamide trifluoroacetate

ESI(pos) 479 [M+H]⁺

### Example 40

### 2-[(2-cyanophenoxy)methyl]-N-[1-(4-fluorobenzyl)-1H-pyrazol-4-yl]morpholine-4-carboxamide trifluoroacetate

ESI(pos) 436 [M+H]⁺

### Example 41

### N-[1-(4-fluorobenzyl)-1H-pyrazol-4-yl]-2-{[2-(trifluoromethoxy)phenoxy]methyl}morpholine-4-carboxamide trifluoroacetate

ESI(pos) 495 [M+H]⁺

### Example 42

### N-[1-(4-fluorobenzyl)-1H-pyrazol-4-yl]-2-{[2-(1-methyl-1H-pyrazol-5-yl)phenoxy]methyl}morpholine-4-carboxamide trifluoroacetate

ESI(pos) 491 [M+H]⁺

### Example 43

### 2-[(3-chloro-4-fluorophenoxy)methyl]-N-[1-(4-fluorobenzyl)-1H-pyrazol-4-yl]morpholine-4-carboxamide trifluoroacetate

ESI(pos) 463 [M+H]⁺

### Example 44

### N-[1-(4-fluorobenzyl)-1H-pyrazol-4-yl]-2-[(2-isoxazol-5-ylphenoxy)methyl]morpholine-4-carboxamide trifluoroacetate

ESI(pos) 478 [M+H]⁺

### Example 45

### N-[1-(4-fluorobenzyl)-1H-pyrazol-4-yl]-2-{[3-(trifluoromethyl)phenoxy]methyl}morpholine-4-carboxamide trifluoroacetate

ESI(pos) 479 [M+H]⁺

### Example 46

### 2-[(4-chlorophenoxy)methyl]-N-[1-(4-fluorobenzyl)-1H-pyrazol-4-yl]morpholine-4-carboxamide trifluoroacetate

ESI(pos) 445 [M+H]⁺

### Example 47

### 2-[(2-bromophenoxy)methyl]-N-[1-(4-fluorobenzyl)-1H-pyrazol-4-yl]morpholine-4-carboxamide trifluoroacetate

ESI(pos) 489 [M+H]⁺

### Example 48

### 2-[(3-bromophenoxy)methyl]-N-[1-(4-fluorobenzyl)-1H-pyrazol-4-yl]morpholine-4-carboxamide trifluoroacetate

ESI(pos) 489 [M+H]⁺

### Example 49

### 2-{[2-chloro-5-(trifluoromethyl)phenoxy]methyl}-N-[1-(4-fluorobenzyl)-1H-pyrazol-4-yl]morpholine-4-carboxamide trifluoroacetate

ESI(pos) 513 [M+H]⁺

### Example 50

### 2-[(2,5-difluorophenoxy)methyl]-N-[1-(4-fluorobenzyl)-1H-pyrazol-4-yl]morpholine-4-carboxamide trifluoroacetate

ESI(pos) 447 [M+H]⁺

### Example 51

### 2-[(2-chloro-6-fluorophenoxy)methyl]-N-[1-(4-fluorobenzyl)-1H-pyrazol-4-yl]morpholine-4-carboxamide trifluoroacetate

ESI(pos) 463 [M+H]⁺

### Example 52

### 2-[(2-chloro-5-fluorophenoxy)methyl]-N-[1-(4-fluorobenzyl)-1H-pyrazol-4-yl]morpholine-4-carboxamide trifluoroacetate

ESI(pos) 463 [M+H]⁺

### Example 53

### 2-{[2-chloro-4-(trifluoromethyl)phenoxy]methyl}-N-[1-(4-fluorobenzyl)-1H-pyrazol-4-yl]morpholine-4-carboxamide trifluoroacetate

ESI(pos) 513 [M+H]⁺

### Example 54

### 2-[(2,4-dichlorophenoxy)methyl]-N-[1-(4-fluorobenzyl)-1H-pyrazol-4-yl]morpholine-4-carboxamide trifluoroacetate

ESI(pos) 479 [M+H]⁺

### Examples 55-80

### (1) N-(4,6-dimethylpyridin-2-yl)-2-(hydroxymethyl)morpholine-4-carboxamide

Using phenyl (4,6-dimethylpyridin-2-yl)carbamate obtained in Example 17(1) and in the same manner as in the synthesis of N-[1-(4-fluorobenzyl)-1H-pyrazol-4-yl]-2-(hydroxymethyl)morpholine-4-carboxamide shown in Example 23, the object compound (67%) was obtained.

¹H NMR (300 MHz, DMSO-d₆) δ ppm 2.22 (s, 3H) 2.32 (s, 3H) 2.62 (dd, J=10.08, 13.09 Hz, 1H) 2.82-2.94 (m, 1H) 3.29-3.49 (m, 4H) 3.82 (dd, J=2.07, 11.49 Hz, 1H) 3.95 (d, J=13.37 Hz, 1H) 4.07 (d, J=13.00 Hz, 1H) 4.75 (t, J=5.56 Hz, 1H) 6.67 (s, 1H) 7.44 (s, 1H) 8.96 (s, 1H)

(2) To a solution of N-(4,6-dimethylpyridin-2-yl)-2-(hydroxymethyl)morpholine-4-carboxamide obtained in the above in tetrahydrofuran were added triphenylphosphine polystyrene resin and various phenols, and then di-tert-butyl azodicarboxylate was added. The mixture was stirred at 50° C. overnight. The reaction solution was filtered, and the solvent was evaporated. The residue was dissolved in a solution (1 mL) of dimethyl sulfoxide/methanol=1/1 and purified by preparative HPLC to give the object compound at a purity of not less than 80% (LCMS analysis).

### Example 55

### N-(4,6-dimethylpyridin-2-yl)-2-(phenoxymethyl)morpholine-4-carboxamide trifluoroacetate

ESI(pos) 342 [M+H]⁺

### Example 56

### 2-[(2,3-dichlorophenoxy)methyl]-N-(4,6-dimethylpyridin-2-yl)morpholine-4-carboxamide trifluoroacetate

ESI(pos) 410 [M+H]⁺

### Example 57

### 2-[(3,5-dichlorophenoxy)methyl]-N-(4,6-dimethylpyridin-2-yl)morpholine-4-carboxamide trifluoroacetate

ESI(pos) 410 [M+H]⁺

### Example 58

### N-(4,6-dimethylpyridin-2-yl)-2-[(3-fluorophenoxy)methyl]morpholine-4-carboxamide trifluoroacetate

ESI(pos) 360 [M+H]⁺

### Example 59

### N-(4,6-dimethylpyridin-2-yl)-2-[(2-fluorophenoxy)methyl]morpholine-4-carboxamide trifluoroacetate

ESI(pos) 360 [M+H]⁺

### Example 60

### 2-{[(2-chloropyridin-3-yl)oxy]methyl}-N-(4,6-dimethylpyridin-2-yl)morpholine-4-carboxamide trifluoroacetate

ESI(pos) 377 [M+H]⁺

### Example 61

### N-(4,6-dimethylpyridin-2-yl)-2-[(pyridin-2-yloxy)methyl]morpholine-4-carboxamide trifluoroacetate

ESI(pos) 343 [M+H]⁺

### Example 62

### 2-{[(6-chloropyridin-2-yl)oxy]methyl}-N-(4,6-dimethylpyridin-2-yl)morpholine-4-carboxamide trifluoroacetate

ESI(pos) 378 [M+H]⁺

### Example 63

### N-(4,6-dimethylpyridin-2-yl)-2-{[(2-methylpyrimidin-4-yl)oxy]methyl}morpholine-4-carboxamide trifluoroacetate

ESI(pos) 358 [M+H]⁺

### Example 64

### N-(4,6-dimethylpyridin-2-yl)-2-{[2-(trifluoromethyl)phenoxy]methyl}morpholine-4-carboxamide trifluoroacetate

ESI(pos) 410 [M+H]⁺

### Example 65

### 2-[(2-cyanophenoxy)methyl]-N-(4,6-dimethylpyridin-2-yl)morpholine-4-carboxamide trifluoroacetate

ESI(pos) 367 [M+H]⁺

### Example 66

### N-(4,6-dimethylpyridin-2-yl)-2-{[2-(trifluoromethoxy)phenoxy]methyl}morpholine-4-carboxamide trifluoroacetate

ESI(pos) 426 [M+H]⁺

### Example 67

### N-(4,6-dimethylpyridin-2-yl)-2-{[2-(1-methyl-1H-pyrazol-5-yl)phenoxy]methyl}morpholine-4-carboxamide trifluoroacetate

ESI(pos) 422 [M+H]⁺

### Example 68

### 2-[(3-chloro-4-fluorophenoxy)methyl]-N-(4,6-dimethylpyridin-2-yl)morpholine-4-carboxamide trifluoroacetate

ESI(pos) 394 [M+H]⁺

### Example 69

### N-(4,6-dimethylpyridin-2-yl)-2-[(2-isoxazol-5-ylphenoxy)methyl]morpholine-4-carboxamide trifluoroacetate

ESI(pos) 409 [M+H]⁺

### Example 70

### N-(4,6-dimethylpyridin-2-yl)-2-{[3-(trifluoromethyl)phenoxy]methyl}morpholine-4-carboxamide trifluoroacetate

ESI(pos) 410 [M+H]⁺

### Example 71

### N-(4,6-dimethylpyridin-2-yl)-2-[(4-fluorophenoxy)methyl]morpholine-4-carboxamide trifluoroacetate

ESI(pos) 360 [M+H]⁺

### Example 72

### 2-[(4-chlorophenoxy)methyl]-N-(4,6-dimethylpyridin-2-yl)morpholine-4-carboxamide trifluoroacetate

ESI(pos) 376 [M+H]⁺

### Example 73

### 2-[(2-bromophenoxy)methyl]-N-(4,6-dimethylpyridin-2-yl)morpholine-4-carboxamide trifluoroacetate

ESI(pos) 420 [M+H]⁺

### Example 74

### 2-[(3-bromophenoxy)methyl]-N-(4,6-dimethylpyridin-2-yl)morpholine-4-carboxamide trifluoroacetate

ESI(pos) 420 [M+H]⁺

### Example 75

### 2-{[2-chloro-5-(trifluoromethyl)phenoxy]methyl}-N-(4,6-dimethylpyridin-2-yl)morpholine-4-carboxamide trifluoroacetate

ESI(pos) 444 [M+H]⁺

### Example 76

### 2-[(2,5-difluorophenoxy)methyl]-N-(4,6-dimethylpyridin-2-yl)morpholine-4-carboxamide trifluoroacetate

ESI(pos) 378 [M+H]⁺

### Example 77

### 2-[(2-chloro-6-fluorophenoxy)methyl]-N-(4,6-dimethylpyridin-2-yl)morpholine-4-carboxamide trifluoroacetate

ESI(pos) 394 [M+H]⁺

### Example 78

### 2-[(2-chloro-5-fluorophenoxy)methyl]-N-(4,6-dimethylpyridin-2-yl)morpholine-4-carboxamide trifluoroacetate

ESI(pos) 394 [M+H]⁺

### Example 79

### 2-{[2-chloro-4-(trifluoromethyl)phenoxy]methyl}-N-(4,6-dimethylpyridin-2-yl)morpholine-4-carboxamide trifluoroacetate

ESI(pos) 444 [M+H]⁺

### Example 80

### 2-[(2,4-dichlorophenoxy)methyl]-N-(4,6-dimethylpyridin-2-yl)morpholine-4-carboxamide trifluoroacetate

ESI(pos) 410 [M+H]⁺

### Examples 81-98

To a solution of 3-[(2,5-dichlorophenoxy)methyl]benzoic acid in tetrahydrofuran was added oxalyl chloride, and a catalytic amount of N,N-dimethylformamide was further added. The reaction mixture was stirred at room temperature for 20 min. The solvent was evaporated under reduced pressure, and the residue was dissolved in N,N-dimethylacetamide. This solution was added to a solution of the corresponding amine derivative in N,N-dimethylacetamide. The reaction mixture was stirred at 60° C. overnight. Ethyl acetate was added, and the mixture was washed with 5% aqueous sodium hydroxide solution. The solvent of the organic layer was evaporated, and the residue was dissolved in dimethylformamide/methanol=1/1 solution (1 mL) and purified by preparative HPLC to give the object compound at a purity of not less than 80% (LCMS analysis).

### Example 81

### 3-[(2,5-dichlorophenoxy)methyl]-N-pyridin-2-ylbenzamide trifluoroacetate

ESI(pos) 373 [M+H]⁺

### Example 82

### 3-[(2,5-dichlorophenoxy)methyl]-N-pyridin-3-ylbenzamide trifluoroacetate

ESI(pos) 373 [M+H]⁺

### Example 83

### 3-[(2,5-dichlorophenoxy)methyl]-N-pyridin-4-ylbenzamide trifluoroacetate

ESI(pos) 373 [M+H]⁺

### Example 84

### 3-[(2,5-dichlorophenoxy)methyl]-N-pyrimidin-2-ylbenzamide trifluoroacetate

ESI(pos) 374 [M+H]⁺

### Example 85

### 3-[(2,5-dichlorophenoxy)methyl]-N-pyrazin-2-ylbenzamide

ESI(pos) 374 [M+H]⁺

### Example 86

### 3-[(2,5-dichlorophenoxy)methyl]-N-(5-methyl-1H-pyrazol-3-yl)benzamide

ESI(pos) 377 [M+H]⁺

### Example 87

### 3-[(2,5-dichlorophenoxy)methyl]-N-(5-methyl-4H-1,2,4-triazol-3-yl)benzamide

ESI(pos) 393 [M+H]⁺

### Example 88

### 3-[(2,5-dichlorophenoxy)methyl]-N-(5-methyl-1,3-thiazol-2-yl)benzamide

ESI(pos) 393 [M+H]⁺

### Example 89

### 3-[(2,5-dichlorophenoxy)methyl]-N-(5-methylisothiazol-3-yl)benzamide

ESI(pos) 378 [M+H]⁺

### Example 90

### 3-[(2,5-dichlorophenoxy)methyl]-N-(4,6-dimethylpyridin-2-yl)benzamide trifluoroacetate

ESI(pos) 401 [M+H]⁺

### Example 91

### N-(6-chloropyridazin-3-yl)-3-[(2,5-dichlorophenoxy)methyl]benzamide

ESI(pos) 408 [M+H]⁺

### Example 92

### 6-({3-[(2,5-dichlorophenoxy)methyl]benzoyl}amino)nicotinamide trifluoroacetate

ESI(pos) 416 [M+H]⁺

### Example 93

### 2-({3-[(2,5-dichlorophenoxy)methyl]benzoyl}amino)thiophene-3-carboxamide

ESI(pos) 421 [M+H]⁺

### Example 94

### N-1,3-benzothiazol-2-yl-3-[(2,5-dichlorophenoxy)methyl]benzamide

ESI(pos) 429 [M+H]⁺

### Example 95

### 3-[(2,5-dichlorophenoxy)methyl]-N-(1-phenyl-1H-pyrazol-5-yl)benzamide

ESI(pos) 438 [M+H]⁺

### Example 96

### 3-[(2,5-dichlorophenoxy)methyl]-N-[1-(2-fluorobenzyl)-1H-pyrazol-3-yl]benzamide

ESI(pos) 470 [M+H]⁺

### Example 97

### N-[3-(benzyloxy)pyridin-2-yl]-3-[(2,5-dichlorophenoxy)methyl]benzamide trifluoroacetate

ESI(pos) 480 [M+H]⁺

### Example 98

### 2-({3-[(2,5-dichlorophenoxy)methyl]benzoyl}amino)-5-(4-fluorophenyl)thiophene-3-carboxamide

ESI(pos) 515 [M+H]⁺

### Example 99

### 4-(1H-benzimidazol-1-ylmethyl)-N-[1-(4-fluorobenzyl)-1H-pyrazol-4-yl]benzamide

Using 4-(1H-benzimidazol-1-ylmethyl)benzoic acid and in the same manner as in Example 2, the object product (38%) was obtained.

¹H NMR (300 MHz, DMSO-d₆) δ ppm 5.29 (s, 2H) 5.58 (s, 2H) 7.12-7.23 (m, 4H) 7.25-7.33 (m, 2H) 7.41 (d, J=8.33 Hz, 2H) 7.49 (d, J=4.92 Hz, 1H) 7.58 (s, 1H) 7.63-7.70 (m, 1H) 7.87 (d, J=8.33 Hz, 2H) 8.12 (s, 1H) 8.44 (s, 1H) 10.40 (s, 1H)

### Example 100

### 3-(phenoxymethyl)-N-[4-(1H-pyrazol-1-ylmethyl)phenyl]-1-benzofuran-2-carboxamide

Using 3-(phenoxymethyl)-1-benzofuran-2-carboxylic acid and in the same manner as the synthesis in Example 1, which is followed by purification by silica gel column chromatography and recrystallization from ethyl acetate/hexane, the object compound (76%) was obtained.

¹H NMR (300 MHz, DMSO-d₆) δ ppm 5.31 (s, 2H) 5.75 (s, 2H) 6.28 (t, J=1.89 Hz, 1H) 6.93 (t, J=7.19 Hz, 1H) 7.07 (d, J=7.57 Hz, 2H) 7.19-7.33 (m, 4H) 7.38 (t, J=7.57 Hz, 1H) 7.47 (d, J=1.89 Hz, 1H) 7.54 (t, J=7.19 Hz, 1H) 7.72 (d, J=8.33 Hz, 1H) 7.77-7.85 (m, 3H) 7.91 (d, J=7.57 Hz, 1H) 10.69 (s, 1H)

### Example 101

### benzyl[2-({[4-(1H-pyrazol-1-ylmethyl)phenyl]amino}carbonyl)-3-thienyl]carbamate

### (1) methyl 3-{[(benzyloxy)carbonyl]amino}thiophene-2-carboxylate

A solution (400 mL) of methyl 3-aminothiophene-2-carboxylate (20.6 g) and benzyl chlorocarbonate (22.4 mL) in toluene was stirred under reflux for 3 hr. After cooling to room temperature, the solvent was evaporated under reduced pressure, and ethyl acetate (400 mL) was added. The mixture was washed 3 times with saturated aqueous sodium hydrogen carbonate solution, and the organic layer was dried over magnesium sulfate. The solvent was evaporated under reduced pressure to give the object product as a crude product. The obtained crude product was used for the next reaction without further purification.

### (2) 3-{[(benzyloxy)carbonyl]amino}thiophene-2-carboxylic acid

Methyl 3-{[(benzyloxy)carbonyl]amino}thiophene-2-carboxylate obtained in the above-mentioned reaction was dissolved in N,N-dimethylformamide/water mixed solvent (3:1, 200 mL), sodium hydroxide (9 g) was added, and the mixture was stirred at room temperature for 3 hr. The reaction solution was acidified with 1N hydrochloric acid, and the crude product was extracted with ethyl acetate. The organic layer was dried over magnesium sulfate, and the solvent was evaporated under reduced pressure. The residue was purified by silica gel column chromatography, and the resultant product was recrystallized from diisopropyl ether to give the object product (16.9 g).

¹H NMR (300 MHz, CDCl₃) δ ppm 5.23 (s, 2H) 7.32-7.46 (m, 5H) 7.55 (d, J=5.4 Hz, 1H) 7.94 (brd, 1H) 9.42 (brs, 1H)

### (3) benzyl[2-({[4-(1H-pyrazol-1-ylmethyl)phenyl]amino}carbonyl)-3-thienyl]carbamate

In the same manner as in Example 100 and using 3-{[(benzyloxy)carbonyl]amino}thiophene-2-carboxylic acid obtained in the above-mentioned reaction, the object compound (66%) was obtained.

¹H NMR (300 MHz, DMSO-d₆) δ ppm 5.18 (s, 2H) 5.29 (s, 2H) 6.26 (t, J=2.08 Hz, 1H) 7.19 (d, J=8.71 Hz, 2H) 7.29-7.48 (m, 6H) 7.60 (d, J=8.33 Hz, 2H) 7.69-7.89 (m, 3H) 10.01 (brs, 1H) 10.46 (brs, 1H)

### Example 102

### 1-benzyl-2,3-dimethyl-N-[4-(1H-pyrazol-1-ylmethyl)phenyl]-1H-indole-6-carboxamide

In the same manner as in Example 100 and using 1-benzyl-2,3-dimethyl-1H-indole-6-carboxylic acid, the object compound (53%) was obtained.

¹H NMR (300 MHz, DMSO-d₆) δ ppm 2.25 (s, 3H) 2.30 (s, 3H) 5.28 (s, 2H) 5.49 (s, 2H) 6.26 (t, J=1.89 Hz, 1H) 6.97 (d, J=6.82 Hz, 2H) 7.15-7.34 (m, 5H) 7.45 (d, J=1.89 Hz, 1H) 7.55 (d, J=8.33 Hz, 1H) 7.70 (t, J=8.71 Hz, 3H) 7.80 (d, J=2.27 Hz, 1H) 8.06 (s, 1H) 10.06 (s, 1H)

### Example 103

### 5-(benzyloxy)-N-[4-(1H-pyrazol-1-ylmethyl)phenyl]-1-benzofuran-2-carboxamide

### (1) ethyl 5-methoxy-1-benzofuran-2-carboxylate

A suspension (100 mL) of 2-hydroxy-5-methoxybenzaldehyde (10 g), ethyl bromoacetate (8.02 mL) and potassium carbonate (18.2 g) in N,N-dimethylformamide was stirred at 80° C. overnight. After cooling to room temperature, ethyl acetate was added, and the mixture was washed 3 times with saturated aqueous sodium carbonate solution. The organic layer was dried over magnesium sulfate, and the solvent was evaporated under reduced pressure. The residue was purified by silica gel column chromatography and recrystallized from ethyl acetate/hexane to give the object product (8.37 g, 58%).

¹H NMR (200 MHz, CDCl₃) δ ppm 1.43 (t, J=7.0 Hz, 3H) 3.85 (s, 3H) 4.44 (q, J=7.0 Hz, 2H) 7.02-7.10 (m, 2H) 7.45-7.51 (m, 2H)

### (2) ethyl 5-hydroxy-1-benzofuran-2-carboxylate

To a solution (7 mL) of ethyl 5-methoxy-1-benzofuran-2-carboxylate (690 mg) obtained in the above-mentioned reaction in toluene was added trichloroborane dimethylsulfide (1.69 g), and the mixture was stirred at 90° C. for 6 hr. After cooling to room temperature, water (20 mL) was added, and the mixture was further stirred at room temperature for 30 min. The mixture was extracted with ethyl acetate and washed 3 times with saturated aqueous sodium hydrogen carbonate solution. The organic layer was dried over sodium sulfate, and the solvent was evaporated under reduced pressure. The residue was purified by silica gel column chromatography and recrystallized from ethyl acetate/hexane to give the object product (330 mg, 64%).

¹H NMR (200 MHz, CDCl₃) δ ppm 1.43 (t, J=7.4 Hz, 3H) 4.44 (q, J=7.4 Hz, 2H) 4.93 (s, 1H) 6.99 (dd, J=2.2, 8.4 Hz, 1H) 7.07 (m, 2H) 7.43 (s, 1H), 7.46 (d, J=8.4 Hz, 1H)

### (3) ethyl 5-(benzyloxy)-1-benzofuran-2-carboxylate

A solution of ethyl 5-hydroxy-1-benzofuran-2-carboxylate (1.5 g) obtained in the above-mentioned reaction, benzyl bromide (1.4 g) and potassium carbonate (1.3 g) in N,N-dimethylformamide (20 mL) was stirred at 50° C. for 24 hr. After cooling, aqueous ammonium chloride solution was added, and the mixture was extracted with ethyl acetate. The organic layer was washed with saturated brine and dried over sodium sulfate, and the solvent was evaporated under reduced pressure. The residue was purified by silica gel column (30% ethyl acetate/hexane to 50% ethyl acetate/hexane) to give the object product (1.8 g) as a white solid.

¹H NMR (300 MHz, CDCl₃) δ ppm 1.42 (t, J=6.9 Hz, 3H) 4.42 (q, J=6.9 Hz, 2H) 5.09 (s, 2H) 7.11-7.16 (m, 2H) 7.33-7.50 (s, 7H)

### (4) 5-(benzyloxy)-1-benzofuran-2-carboxylic acid

Ethyl 5-(benzyloxy)-1-benzofuran-2-carboxylate (1.7 g) obtained in the above-mentioned reaction was dissolved in tetrahydrofuran/methanol (1:1, 40 mL) mixed solvent. 1N Aqueous sodium hydroxide solution (80 mL) was added, and the mixture was stirred at room temperature overnight. 1N Hydrochloric acid (80 mL) was added, and the mixture was further stirred at room temperature for 2 hr. The resulting precipitate was collected by filtration and dried under reduced pressure to give the object product (1.52 g).

¹H NMR (300 MHz, DMSO-d₆) δ ppm 5.15 (s, 2H) 7.18 (dd, J=2.46, 8.90 Hz, 1H) 7.29-7.51 (m, 6H) 7.58 (s, 1H) 7.62 (d, J=9.09 Hz, 1H) 13.54 (brs, 1H)

### (5) 5-(benzyloxy)-N-[4-(1H-pyrazol-1-ylmethyl)phenyl]-1-benzofuran-2-carboxamide

In the same manner as in Example 100 and using 5-(benzyloxy)-1-benzofuran-2-carboxylic acid obtained in the above-mentioned reaction, the object compound (74%) was obtained.

¹H NMR (300 MHz, DMSO-d₆) δ ppm 5.16 (s, 2H) 5.30 (s, 2H) 6.27 (t, J=2.08 Hz, 1H) 7.13-7.27 (m, 3H) 7.30-7.52 (m, 7H) 7.63 (d, J=9.09 Hz, 1H) 7.69 (s, 1H) 7.76 (d, J=8.33 Hz, 2H) 7.81 (d, J=2.27 Hz, 1H) 10.52 (s, 1H)

### Example 104

benzyl(2-{[(5-methylisoxazol-3-yl)amino]carbonyl}-3-thienyl)carbamate

In the same manner as in Example 2 and using 3-{[(benzyloxy)carbonyl]amino}thiophene-2-carboxylic acid obtained in Example 101(2) and 5-methylisoxazol-3-amine, the object compound (58%) was obtained.

¹H NMR (300 MHz, DMSO-d₆) δ ppm 2.39 (d, J=0.75 Hz, 3H) 5.20 (s, 2H) 6.68 (d, J=0.75 Hz, 1H) 7.31-7.48 (m, 5H) 7.76 (d, J=5.46 Hz, 1H) 7.89 (d, J=5.46 Hz, 1H) 10.37 (brs, 1H) 11.16 (brs, 1H)

### Example 105

### 5-(benzyloxy)-N-(5-methylisoxazol-3-yl)-1-benzofuran-2-carboxamide

In the same manner as in Example 104 and using 5-(benzyloxy)-1-benzofuran-2-carboxylic acid, the object compound (38%) was obtained.

¹H NMR (300 MHz, DMSO-d₆) δ ppm 2.42 (s, 3H) 5.16 (s, 2H) 6.75 (s, 1H) 7.19 (dd, J=2.65, 9.09 Hz, 1H) 7.30-7.45 (m, 4H) 7.46-7.54 (m, 2H) 7.63 (d, J=9.09 Hz, 1H) 7.88 (s, 1H) 11.55 (brs, 1H)

### Example 106

### 4-ethoxy-3-[2-(4-fluorophenyl)ethyl]-N-(5-methylisoxazol-3-yl)benzamide

### (1) 4-ethoxy-3-[2-(4-fluorophenyl)vinyl]benzoic acid

A suspension of 3-bromo-4-ethoxybenzoic acid (4.9 g), 1-fluoro-4-vinylbenzene (4.79 mL), dichlorobis(triphenylphosphine)palladium(II) (414 mg) and triethylamine (5.55 mL) in N,N-dimethylformamide (100 mL) was stirred at 100° C. for 20 min under microwave irradiation. After cooling to room temperature, ethyl acetate was added, and the mixture was washed 3 times with saturated aqueous sodium hydrogen carbonate solution. The organic layer was dried over sodium sulfate, and the solvent was evaporated under reduced pressure. The residue was purified by silica gel column chromatography to give the object product as a crude product. The obtained crude product was used for the next reaction without further purification.

### (2) 4-ethoxy-3-[2-(4-fluorophenyl)ethyl]benzoic acid

A suspension of 4-ethoxy-3-[2-(4-fluorophenyl)vinyl]benzoic acid (1.8 g) obtained in the above-mentioned reaction and 10% palladium/carbon (300 mg) in methanol (100 mL) was stirred at room temperature overnight under a hydrogen atmosphere. The reaction mixture was filtered through celite, and the solvent of the filtrate was evaporated under reduced pressure. The residue was purified by silica gel column chromatography to give the object product as a crude product. The crude product was recrystallized from ethyl acetate/hexane to give the object product (1.4 g).

¹H NMR (300 MHz, DMSO-d₆) δ ppm 1.37 (t, J=6.97 Hz, 3H) 2.76-2.88 (m, 4H) 4.10 (q, J=6.84 Hz, 2H) 6.98-7.13 (m, 3H) 7.16-7.26 (m, 2H) 7.71 (d, J=2.07 Hz, 1H) 7.78 (dd, J=2.17, 8.57 Hz, 1H) 12.53 (brs, 1H)

### (3) 4-ethoxy-3-[2-(4-fluorophenyl)ethyl]-N-(5-methylisoxazol-3-yl)benzamide

In the same manner as in Example 104 and using 4-ethoxy-3-[2-(4-fluorophenyl)ethyl]benzoic acid obtained in the above-mentioned reaction, the object compound (51%) was obtained.

¹H NMR (300 MHz, DMSO-d₆) δ ppm 1.37 (t, J=7.00 Hz, 3H) 2.41 (s, 3H) 2.86 (s, 4H) 4.11 (q, J=6.94 Hz, 2H) 6.74 (s, 1H) 7.00-7.16 (m, 3H) 7.18-7.32 (m, 2H) 7.82-7.96 (m, 2H) 11.09 (s, 1H)

### Example 107

### 4-{[2-(1,3,4-oxadiazol-2-yl)phenoxy]methyl}-N-pyridin-2-ylbenzamide

### (1) 4-(chloromethyl)-N-pyridin-2-ylbenzamide

Using 4-(chloromethyl)benzoic acid, pyridin-2-amine, and in the same manner as in the synthesis of 3-[(2,5-dichlorophenoxy)methyl]-N-[1-(4-fluorobenzyl)-1H-pyrazol-4-yl]benzamide shown in Example 2, the object product (32%) was obtained.

¹H NMR (300 MHz, DMSO-d₆) δ ppm 4.85 (s, 2H) 7.13-7.22 (m, 1H) 7.57 (d, J=8.48 Hz, 2H) 7.80-7.90 (m, 1H) 7.99-8.07 (m, 2H) 8.19 (d, J=8.29 Hz, 1H) 8.36-8.42 (m, 1H) 10.82 (s, 1H)

### (2) 4-{[2-(1,3,4-oxadiazol-2-yl)phenoxy]methyl}-N-pyridin-2-ylbenzamide

Using 4-(chloromethyl)-N-pyridin-2-ylbenzamide obtained in the above-mentioned reaction and 2-(1,3,4-oxadiazol-2-yl)phenol and in the same manner as in Example 3, the object compound (26%) was obtained.

¹H NMR (300 MHz, DMSO-d₆) δ ppm 5.41 (s, 2H) 7.14-7.22 (m, 2H) 7.37 (d, J=7.91 Hz, 1H) 7.57-7.69 (m, 3H) 7.81-7.88 (m, 1H) 7.91 (dd, J=1.70, 7.72 Hz, 1H) 8.06 (d, J=8.29 Hz, 2H) 8.19 (d, J=8.29 Hz, 1H) 8.35-8.42 (m, 1H) 9.38 (s, 1H) 10.78 (s, 1H)

### Example 108

### 4-(1H-benzimidazol-1-ylmethyl)-N-(4,6-dimethylpyridin-2-yl)benzamide

In the same manner as in Example 13 and using 4-(1H-benzimidazol-1-ylmethyl)benzoic acid (252 mg), the object compound (22%) was obtained.

¹H NMR (300 MHz, DMSO-d₆) δ ppm 2.29 (s, 3H) 2.38 (s, 3H) 5.60 (s, 2H) 6.85 (s, 1H) 7.16-7.26 (m, 2H) 7.39 (d, J=8.33 Hz, 2H) 7.48-7.56 (m, 1H) 7.63-7.72 (m, 1H) 7.85 (s, 1H) 7.98 (d, J=8.33 Hz, 2H) 8.46 (s, 1H) 10.59 (s, 1H)

### Example 109

### N-(5-methylisoxazol-3-yl)-3-(phenoxymethyl)-1-benzofuran-2-carboxamide

In the same manner as in Example 104 and using 3-(phenoxymethyl)-1-benzofuran-2-carboxylic acid, the object compound (83%) was obtained.

¹H NMR (300 MHz, DMSO-d₆) δ ppm 2.43 (s, 3H) 5.72 (s, 2H) 6.75 (s, 1H) 6.94 (t, J=7.19 Hz, 1H) 7.06 (d, J=7.57 Hz, 2H) 7.24-7.34 (m, 2H) 7.39 (t, J=7.57 Hz, 1H) 7.50-7.60 (m, 1H) 7.65-7.72 (m, 1H) 7.92 (d, J=7.57 Hz, 1H) 11.60 (s, 1H)

### Example 110

### 1-(2-chlorobenzyl)-N-(4,6-dimethylpyridin-2-yl)-2,3-dimethyl-1H-indole-5-carboxamide

In the same manner as in Example 13 and using 1-(2-chlorobenzyl)-2,3-dimethyl-1H-indole-5-carboxylic acid, the object compound (83%) was obtained.

¹H NMR (300 MHz, DMSO-d₆) δ ppm 2.25 (s, 3H) 2.28-2.34 (m, 6H) 2.41 (s, 3H) 5.49 (s, 2H) 6.14 (dd, J=1.32, 7.72 Hz, 1H) 6.84 (s, 1H) 7.11-7.21 (m, 1H) 7.24-7.33 (m, 1H) 7.36 (d, J=8.67 Hz, 1H) 7.53 (dd, J=1.13, 7.91 Hz, 1H) 7.76 (dd, J=1.70, 8.67 Hz, 1H) 7.92 (s, 1H) 8.39 (d, J=1.51 Hz, 1H) 10.41 (s, 1H)

### Example 111

### 5-(benzyloxy)-N-(4,6-dimethylpyridin-2-yl)-1-benzofuran-2-carboxamide

In the same manner as in Example 13 and using 5-(benzyloxy)-1-benzofuran-2-carboxylic acid obtained in Example 103(4), the object compound (22%) was obtained.

¹H NMR (300 MHz, DMSO-d₆) δ ppm 2.32 (s, 3H) 2.41 (s, 3H) 5.16 (s, 2H) 6.90 (s, 1H) 7.17 (dd, J=2.65, 9.09 Hz, 1H) 7.29-7.45 (m, 4H) 7.46-7.53 (m, 2H) 7.64 (d, J=9.09 Hz, 1H) 7.84 (s, 1H) 7.94 (s, 1H) 10.60 (s, 1H)

### Example 112

### 4-[(2,5-dichlorophenoxy)methyl]-N-[4-(1H-pyrazol-1-ylmethyl)phenyl]pyridine-2-carboxamide

### (1) ethyl 4-[(2,5-dichlorophenoxy)methyl]pyridine-2-carboxylate

Using ethyl 4-(hydroxymethyl)pyridine-2-carboxylate, 2,5-dichlorophenol and in the same manner as in the synthesis of N-tert-butoxycarbonyl-3-[(2-chlorophenoxy)methyl]piperidine shown in Example 15, the object product was obtained as a crude product. The obtained crude product was used for the next reaction without further purification.

### (2) 4-[(2,5-dichlorophenoxy)methyl]pyridine-2-carboxylic acid

Ethyl 4-[(2,5-dichlorophenoxy)methyl]pyridine-2-carboxylate (6.0 g) obtained in the above-mentioned reaction was dissolved in tetrahydrofuran-methanol (1:1, 100 mL) mixed solvent. 1N Aqueous sodium hydroxide solution (80 mL) was added, and the mixture was stirred at room temperature overnight. 1N Hydrochloric acid (80 mL) was added, and the mixture was further stirred at room temperature for 2 hr. The resulting precipitate was collected by filtration and dried under reduced pressure to give the object product (5.9 g).

¹H NMR (300 MHz, DMSO-d₆) δ ppm 5.42 (s, 2H) 7.09 (dd, J=2.26, 8.48 Hz, 1H) 7.36 (d, J=2.26 Hz, 1H) 7.52 (d, J=8.48 Hz, 1H) 7.57-7.62 (m, 1H) 8.11 (s, 1H) 8.67 (d, J=4.90 Hz, 1H)

### (3) 4-[(2,5-dichlorophenoxy)methyl]-N-[4-(1H-pyrazol-1-ylmethyl)phenyl]pyridine-2-carboxamide

Using 4-[(2,5-dichlorophenoxy)methyl]pyridine-2-carboxylic acid obtained in the above-mentioned reaction and in the same manner as in Example 1, the object product (82%) was obtained.

¹H NMR (300 MHz, DMSO-d₆) δ ppm 5.30 (s, 2H) 5.48 (s, 2H) 6.27 (t, J=2.08 Hz, 1H) 7.10 (dd, J=2.27, 8.33 Hz, 1H) 7.23 (d, J=8.71 Hz, 2H) 7.38 (d, J=2.27 Hz, 1H) 7.46 (d, J=1.51 Hz, 1H) 7.53 (d, J=8.33 Hz, 1H) 7.71 (dd, J=1.51, 4.92 Hz, 1H) 7.81 (d, J=2.27 Hz, 1H) 7.86 (d, J=8.33 Hz, 2H) 8.24 (s, 1H) 8.77 (d, J=5.30 Hz, 1H) 10.69 (s, 1H)

### Example 113

### 4-[(2,5-dichlorophenoxy)methyl]-N-(4,6-dimethylpyridin-2-yl)pyridine-2-carboxamide

A solution of 4-[(2,5-dichlorophenoxy)methyl]pyridine-2-carboxylic acid (596 mg) obtained in Example 112(2), 4,6-dimethylpyridin-2-amine (269 mg), o-(benzotriazol-1-yl)-N,N,N,N′-tetramethyluroniumtetrafluoroborate (770 mg) and triethylamine (0.422 mL) in N,N-dimethylformamide (50 mL) was stirred at room temperature overnight. Ethyl acetate was added, the mixture was washed 3 times with saturated aqueous sodium hydrogen carbonate solution, and the organic layer was dried over sodium sulfate. The solvent was evaporated under reduced pressure, and ethyl acetate/hexane was added. The resulting precipitate was collected by filtration, and recrystallized from ethyl acetate/hexane to give the object product (196 mg, 24%).

¹H NMR (300 MHz, DMSO-d₆) δ ppm 2.34 (s, 3H) 2.40 (s, 3H) 5.50 (s, 2H) 6.92 (s, 1H) 7.11 (dd, J=2.27, 8.71 Hz, 1H) 7.39 (d, J=2.27 Hz, 1H) 7.54 (d, J=8.33 Hz, 1H) 7.75 (dd, J=1.51, 4.92 Hz, 1H) 7.95 (s, 1H) 8.29 (s, 1H) 8.78 (d, J=4.92 Hz, 1H) 10.30 (s, 1H)

### Example 114

### 4-[(2,5-dichlorophenoxy)methyl]-N-[1-(4-fluorobenzyl)-1H-pyrazol-4-yl]pyridine-2-carboxamide

Using 4-[(2,5-dichlorophenoxy)methyl]pyridine-2-carboxylic acid obtained in Example 112(2), 1-(4-fluorobenzyl)-1H-pyrazol-4-amine hydrochloride and in the same manner as in Example 113, the object product (66%) was obtained.

¹H NMR (300 MHz, DMSO-d₆) δ ppm 5.31 (s, 2H) 5.47 (s, 2H) 7.09 (dd, J=2.35, 8.57 Hz, 1H) 7.13-7.23 (m, 2H) 7.25-7.35 (m, 2H) 7.38 (d, J=2.26 Hz, 1H) 7.53 (d, J=8.48 Hz, 1H) 7.68 (dd, J=1.70, 4.90 Hz, 1H) 7.79 (s, 1H) 8.18-8.21 (m, 1H) 8.24 (s, 1H) 8.71-8.76 (m, 1H) 10.99 (s, 1H)

### Example 115

### 6-[(2,5-dichlorophenoxy)methyl]-N-[4-(1H-pyrazol-1-ylmethyl)phenyl]pyridine-2-carboxamide

### (1) ethyl 6-({[4-(1H-pyrazol-1-ylmethyl)phenyl]amino}carbonyl)pyridine-2-carboxylate

By reaction in the same manner as in Example 1 and using 6-(ethoxycarbonyl)pyridine-2-carboxylic acid (15.0 g) and 4-(1H-pyrazol-1-ylmethyl)aniline (0.89 g), the object product (12 g) was obtained as a white solid.

¹H NMR (300 MHz, CDCl₃) δ ppm 1.48 (t, J=7.2 Hz, 3H) 4.50 (q, J=7.2 Hz, 2H) 5.32 (s, 2H) 6.28 (t, J=2.4 Hz, 1H) 7.25 (d, J=8.7 Hz, 2H) 7.39 (t, J=1.5 Hz, 1H) 7.55 (s, 1H) 7.76 (d, J=8.7 Hz, 2H) 8.06 (t, J=7.8 Hz, 1H) 8.26 (d, J=8.1 Hz, 1H) 8.46 (d, J=8.1 Hz, 1H) 10.0 (brs, 1H)

### (2) 6-(hydroxymethyl)-N-[4-(1H-pyrazol-1-ylmethyl)phenyl]pyridine-2-carboxamide

To a solution of ethyl 6-({[4-(1H-pyrazol-1-ylmethyl)phenyl]amino}carbonyl)pyridine-2-carboxylate (250 mg) obtained in the above-mentioned reaction and sodium borohydride (60 mg) in ethanol (5 mL) was added calcium chloride (90 mg) under ice-cooling, and the mixture was stirred at room temperature for 1 hr. The reaction mixture was filtered through silica gel, and the filtrate was evaporated under reduced pressure. Ethyl acetate was added to the residue, and the mixture was washed with saturated brine and dried over sodium sulfate. The solvent was evaporated under reduced pressure, and the residue was crystallized from ethyl acetate/hexane and recrystallized from ethyl acetate/hexane to give the object product (110 mg) as a white solid.

¹H NMR (300 MHz, CDCl₃) δ ppm 2.96 (t, J=5.7 Hz, 1H) 4.86 (d, J=5.7 Hz, 2H) 5.31 (s, 2H) 6.28 (t, J=2.4 Hz, 1H) 7.24 (d, J=8.7 Hz, 2H) 7.39 (t, J=1.5 Hz, 1H) 7.54-7.57 (m, 2H) 7.70 (d, J=8.7 Hz, 2H) 7.91 (t, J=7.8 Hz, 1H) 8.18 (d, J=8.1 Hz, 1H) 9.84 (brs, 1H)

### (3) 6-[(2,5-dichlorophenoxy)methyl]-N-[4-(1H-pyrazol-1-ylmethyl)phenyl]pyridine-2-carboxamide

By reaction in the same manner as in Example 15(2) and using 6-(hydroxymethyl)-N-[4-(1H-pyrazol-1-ylmethyl)phenyl]pyridine-2-carboxamide (200 mg) obtained in the above-mentioned reaction and 2,5-dichlorophenol (105 mg), the object product (190 mg) was obtained as a white solid.

¹H NMR (300 MHz, CDCl₃) δ ppm 5.32 (s, 4H) 6.28 (t, J=2.4 Hz, 1H) 6.95 (dd, J=2.1, 8.7 Hz, 1H) 7.03 (d, J=2.1 Hz, 1H) 7.26 (d, J=8.7 Hz, 2H) 7.34 (d, J=8.7 Hz, 1H) 7.39 (d, J=2.4 Hz, 1H) 7.55 (d, J=2.4 Hz, 1H) 7.75 (d, J=8.7 Hz, 2H) 7.80 (d, J=5.2 Hz, 1H) 7.98 (t, J=7.8 Hz, 1H) 8.25 (d, J=5.2 Hz, 1H) 9.90 (brs, 1H)

### Example 116

### 2-[(2,5-dichlorophenoxy)methyl]-N-[4-(1H-pyrazol-1-ylmethyl)phenyl]isonicotinamide

### (1) methyl 2-[(2,5-dichlorophenoxy)methyl]isonicotinate

By reaction in the same manner as in Example 15(2) and using methyl 2-(hydroxymethyl)isonicotinate (800 mg) and 2,5-dichlorophenol (780 mg), the object product (1.2 g) was obtained as a white solid.

¹H NMR (300 MHz, CDCl₃) δ ppm 3.97 (s, 3H) 5.29 (s, 2H) 6.92 (dd, J=2.1, 8.4 Hz, 1H) 6.99 (d, J=2.1 Hz, 1H) 7.31 (d, J=8.7 Hz, 1H) 7.80 (m, 1H) 8.16 (s, 1H) 8.75 (dd, J=0.9, 5.1 Hz, 1H)

### (2) 2-[(2,5-dichlorophenoxy)methyl]isonicotinic acid

By reaction in the same manner as in Example 112(2) and using methyl 2-[(2,5-dichlorophenoxy)methyl]isonicotinate (1.2 g) obtained in the above-mentioned reaction and 4N aqueous sodium hydroxide solution (20 mL), the object product (0.97 g) was obtained as a white solid.

¹H NMR (300 MHz, DMSO-d₆) δ ppm 5.41 (s, 2H) 7.07 (dd, J=2.4, 8.7 Hz, 1H) 7.41 (s, 1H) 7.50 (d, J=8.7 Hz, 1H) 7.79 (dd, J=1.8, 5.1 Hz, 1H) 8.02 (s, 1H) 8.78 (d, J=4.2 Hz, 1H) 14.4 (brs, 1H)

### (3) 2-[(2,5-dichlorophenoxy)methyl]-N-[4-(1H-pyrazol-1-ylmethyl)phenyl]isonicotinamide

By reaction in the same manner as in Example 1 and using 2-[(2,5-dichlorophenoxy)methyl]isonicotinic acid (200 mg) obtained in the above-mentioned reaction and 4-(1H-pyrazol-1-ylmethyl)aniline (116 mg), the object product (180 mg) was obtained as a white solid.

¹H NMR (300 MHz, CDCl₃) δ ppm 5.29 (s, 2H) 5.30 (s, 2H) 6.28 (t, J=2.1 Hz, 1H) 6.93 (dd, J=2.1, 8.4 Hz, 1H) 7.00 (d, J=2.1 Hz, 1H) 7.19 (d, J=8.7 Hz, 2H) 7.31 (d, J=8.4 Hz, 1H) 7.39 (d, J=2.1 Hz, 1H) 7.55 (d, J=1.8 Hz, 1H) 7.58 (d, J=8.7 Hz, 2H) 7.67 (dd, J=1.5, 5.1 Hz, 1H) 7.80 (s, 1H) 8.07 (s, 1H) 8.74 (d, J=5.1 Hz, 1H)

### Example 117

### 2-[(2,5-dichlorophenoxy)methyl]-N-(4,6-dimethylpyridin-2-yl)isonicotinamide

By reaction in the same manner as in Example 113 and using 2-[(2,5-dichlorophenoxy)methyl]isonicotinic acid (300 mg) obtained in Example 116(2) and 4,6-dimethylpyridin-2-amine (125 mg), the object compound (260 mg) was obtained as a white solid.

¹H NMR (300 MHz, CDCl₃) δ ppm 2.37 (s, 3H) 2.42 (s, 3H) 5.31 (s, 2H) 6.81 (s, 1H) 6.93 (dd, J=2.4, 8.4 Hz, 1H) 7.00 (d, J=2.4 Hz, 1H) 7.32 (d, J=8.4 Hz, 1H) 7.70 (dd, J=1.5, 4.8 Hz, 1H) 7.99 (s, 1H) 7.58 (d, J=8.7 Hz, 2H) 7.67 (dd, J=1.5, 5.1 Hz, 1H) 7.80 (s, 1H) 8.07 (s, 1H) 8.57 (brs, 1H), 8.77 (dd, J=0.6, 4.8 Hz, 1H)

### Example 118

### 1-benzyl-3-methyl-N-[4-(1H-pyrazol-1-ylmethyl)phenyl]-1H-thieno[2,3-c]pyrazole-5-carboxamide

Using 1-benzyl-3-methyl-1H-thieno[2,3-c]pyrazole-5-carboxylic acid and in the same manner as in Example 1, the object product (92%) was obtained.

¹H NMR (300 MHz, DMSO-d₆) δ ppm 2.41 (s, 3H) 5.28 (s, 2H) 5.40 (s, 2H) 6.26 (t, J=2.08 Hz, 1H) 7.20 (d, J=8.71 Hz, 2H) 7.30-7.51 (m, 6H) 7.64 (d, J=8.33 Hz, 2H) 7.80 (d, J=2.27 Hz, 1H) 8.03 (s, 1H) 10.22 (s, 1H)

### Example 119

### 4-[(2-chlorophenoxy)methyl]-N-[4-(1H-pyrazol-1-ylmethyl)phenyl]pyridine-2-carboxamide

### (1) ethyl 4-[(2-chlorophenoxy)methyl]pyridine-2-carboxylate

Using ethyl 4-(hydroxymethyl)pyridine-2-carboxylate, 2-chlorophenol and in the same manner as in the synthesis of N-tert-butoxycarbonyl-3-[(2-chlorophenoxy)methyl]piperidine shown in Example 15, the object product was obtained as a crude product. The obtained crude product was used for the next reaction without further purification.

### (2) 4-[(2-chlorophenoxy)methyl]pyridine-2-carboxylic acid

Using ethyl 4-[(2-chlorophenoxy)methyl]pyridine-2-carboxylate obtained in the above-mentioned reaction and in the same manner as in the synthesis of 4-[(2,5-dichlorophenoxy)methyl]pyridine-2-carboxylic acid shown in Example 112, the object product was obtained as a crude product. The obtained crude product was used for the next reaction without further purification.

### (3) 4-[(2-chlorophenoxy)methyl]-N-[4-(1H-pyrazol-1-ylmethyl)phenyl]pyridine-2-carboxamide

Using 4-[(2-chlorophenoxy)methyl]pyridine-2-carboxylic acid obtained in the above-mentioned reaction and in the same manner as in Example 112, the object product (70%) was obtained.

¹H NMR (300 MHz, DMSO-d₆) δ ppm 5.30 (s, 2H) 5.44 (s, 2H) 6.27 (t, J=2.07 Hz, 1H) 6.95-7.06 (m, 1H) 7.18-7.26 (m, 3H) 7.26-7.36 (m, 1H) 7.44-7.52 (m, 2H) 7.72 (dd, J=1.60, 4.99 Hz, 1H) 7.81 (d, J=1.70 Hz, 1H) 7.86 (d, J=8.48 Hz, 2H) 8.25 (s, 1H) 8.76 (d, J=5.09 Hz, 1H) 10.68 (s, 1H)

### Example 120

### 4-[(3-chlorophenoxy)methyl]-N-[4-(1H-pyrazol-1-ylmethyl)phenyl]pyridine-2-carboxamide

### (1) ethyl 4-[(3-chlorophenoxy)methyl]pyridine-2-carboxylate

Using ethyl 4-(hydroxymethyl)pyridine-2-carboxylate, 3-chlorophenol and in the same manner as in the synthesis of N-tert-butoxycarbonyl-3-[(2-chlorophenoxy)methyl]piperidine shown in Example 15, the object product was obtained as a crude product. The obtained crude product was used for the next reaction without further purification.

### (2) 4-[(3-chlorophenoxy)methyl]pyridine-2-carboxylic acid

Using ethyl 4-[(3-chlorophenoxy)methyl]pyridine-2-carboxylate obtained in the above-mentioned reaction and in the same manner as in the synthesis of 4-[(2,5-dichlorophenoxy)methyl]pyridine-2-carboxylic acid shown in Example 112, the object product was obtained as a crude product. The obtained crude product was used for the next reaction without further purification.

### (3) 4-[(3-chlorophenoxy)methyl]-N-[4-(1H-pyrazol-1-ylmethyl)phenyl]pyridine-2-carboxamide

Using 4-[(3-chlorophenoxy)methyl]pyridine-2-carboxylic acid obtained in the above-mentioned reaction and in the same manner as in Example 112, the object product (81%) was obtained.

¹H NMR (300 MHz, DMSO-d₆) δ ppm 5.30 (s, 2H) 5.37 (s, 2H) 6.27 (t, J=1.98 Hz, 1H) 7.00-7.08 (m, 2H) 7.17 (t, J=2.17 Hz, 1H) 7.23 (d, J=8.48 Hz, 2H) 7.35 (t, J=8.19 Hz, 1H) 7.46 (d, J=1.13 Hz, 1H) 7.71 (dd, J=1.60, 4.99 Hz, 1H) 7.80-7.83 (m, 1H) 7.86 (d, J=8.67 Hz, 2H) 8.20 (d, J=0.94 Hz, 1H) 8.70-8.80 (m, 1H) 10.68 (s, 1H)

### Example 121

### 4-[(2-chloro-4-fluorophenoxy)methyl]-N-[4-(1H-pyrazol-1-ylmethyl)phenyl]pyridine-2-carboxamide

### (1) ethyl 4-[(2-chloro-4-fluorophenoxy)methyl]pyridine-2-carboxylate

Using ethyl 4-(hydroxymethyl)pyridine-2-carboxylate and 2-chloro-4-fluorophenol and in the same manner as in the synthesis of N-tert-butoxycarbonyl-3-[(2-chlorophenoxy)methyl]piperidine shown in Example 15, the object product was obtained as a crude product. The obtained crude product was used for the next reaction without further purification.

### (2) 4-[(2-chloro-4-fluorophenoxy)methyl]pyridine-2-carboxylic acid

Using ethyl 4-[(2-chloro-4-fluorophenoxy)methyl]pyridine-2-carboxylate obtained in the above-mentioned reaction and in the same manner as in the synthesis of 4-[(2,5-dichlorophenoxy)methyl]pyridine-2-carboxylic acid shown in Example 112, the object product was obtained as a crude product. The obtained crude product was used for the next reaction without further purification.

### (3) 4-[(2-chloro-4-fluorophenoxy)methyl]-N-[4-(1H-pyrazol-1-ylmethyl)phenyl]pyridine-2-carboxamide

Using 4-[(2-chloro-4-fluorophenoxy)methyl]pyridine-2-carboxylic acid obtained in the above-mentioned reaction and in the same manner as in Example 112, the object product (63%) was obtained.

¹H NMR (300 MHz, DMSO-d₆) δ ppm 5.31 (s, 2H) 5.42 (s, 2H) 6.27 (t, J=2.07 Hz, 1H) 7.17-7.29 (m, 4H) 7.44-7.55 (m, 2H) 7.71 (dd, J=1.70, 4.90 Hz, 1H) 7.82 (d, J=1.70 Hz, 1H) 7.87 (d, J=8.67 Hz, 2H) 8.25 (d, J=0.94 Hz, 1H) 8.76 (d, J=5.65 Hz, 1H) 10.68 (s, 1H)

### Example 122

### 4-[(2-fluorophenoxy)methyl]-N-[4-(1H-pyrazol-1-ylmethyl)phenyl]pyridine-2-carboxamide

### (1) ethyl 4-[(2-fluorophenoxy)methyl]pyridine-2-carboxylate

Using ethyl 4-(hydroxymethyl)pyridine-2-carboxylate, 2-fluorophenol and in the same manner as in the synthesis of N-tert-butoxycarbonyl-3-[(2-chlorophenoxy)methyl]piperidine shown in Example 15, the object product was obtained as a crude product. The obtained crude product was used for the next reaction without further purification.

### (2) 4-[(2-fluorophenoxy)methyl]pyridine-2-carboxylic acid

Using ethyl 4-[(2-fluorophenoxy)methyl]pyridine-2-carboxylate obtained in the above-mentioned reaction and in the same manner as in the synthesis of 4-[(2,5-dichlorophenoxy)methyl]pyridine-2-carboxylic acid shown in Example 112, the object product was obtained as a crude product. The obtained crude product was used for the next reaction without further purification.

### (3) 4-[(2-fluorophenoxy)methyl]-N-[4-(1H-pyrazol-1-ylmethyl)phenyl]pyridine-2-carboxamide

Using 4-[(2-fluorophenoxy)methyl]pyridine-2-carboxylic acid obtained in the above-mentioned reaction and in the same manner as in Example 112, the object product (75%) was obtained.

¹H NMR (300 MHz, DMSO-d₆) δ ppm 5.30 (s, 2H) 5.41 (s, 2H) 6.27 (t, J=2.07 Hz, 1H) 6.93-7.04 (m, 1H) 7.10-7.18 (m, 1H) 7.19-7.32 (m, 4H) 7.46 (d, J=1.32 Hz, 1H) 7.71 (dd, J=1.60, 4.99 Hz, 1H) 7.81 (d, J=1.70 Hz, 1H) 7.86 (d, J=8.48 Hz, 2H) 8.22 (d, J=0.75 Hz, 1H) 8.75 (d, J=5.09 Hz, 1H) 10.68 (s, 1H)

### Example 123

### 4-[(3-fluorophenoxy)methyl]-N-[4-(1H-pyrazol-1-ylmethyl)phenyl]pyridine-2-carboxamide

### (1) ethyl 4-[(3-fluorophenoxy)methyl]pyridine-2-carboxylate

Using ethyl 4-(hydroxymethyl)pyridine-2-carboxylate, 3-fluorophenol and in the same manner as in the synthesis of N-tert-butoxycarbonyl-3-[(2-chlorophenoxy)methyl]piperidine shown in Example 15, the object product was obtained as a crude product. The obtained crude product was used for the next reaction without further purification.

### (2) 4-[(3-fluorophenoxy)methyl]pyridine-2-carboxylic acid

Using ethyl 4-[(3-fluorophenoxy)methyl]pyridine-2-carboxylate obtained in the above-mentioned reaction and in the same manner as in the synthesis of 4-[(2,5-dichlorophenoxy)methyl]pyridine-2-carboxylic acid shown in Example 112, the object product was obtained as a crude product. The obtained crude product was used for the next reaction without further purification.

### (3) 4-[(3-fluorophenoxy)methyl]-N-[4-(1H-pyrazol-1-ylmethyl)phenyl]pyridine-2-carboxamide

Using 4-[(3-fluorophenoxy)methyl]pyridine-2-carboxylic acid obtained in the above-mentioned reaction and in the same manner as in Example 112, the object product (50%) was obtained.

¹H NMR (300 MHz, DMSO-d₆) δ ppm 5.31 (s, 2H) 5.36 (s, 2H) 6.27 (t, J=1.98 Hz, 1H) 6.77-6.87 (m, 1H) 6.86-7.02 (m, 2H) 7.23 (d, J=8.67 Hz, 2H) 7.30-7.41 (m, 1H) 7.46 (d, J=1.32 Hz, 1H) 7.71 (dd, J=1.60, 4.99 Hz, 1H) 7.81 (d, J=1.70 Hz, 1H) 7.86 (d, J=8.67 Hz, 2H) 8.20 (s, 1H) 8.74 (d, J=5.09 Hz, 1H) 10.68 (s, 1H)

### Example 124

### 4-[(2-chlorophenoxy)methyl]-N-(4,6-dimethylpyridin-2-yl)pyridine-2-carboxamide

Using 4-[(2-chlorophenoxy)methyl]pyridine-2-carboxylic acid obtained in Example 119(2) and in the same manner as in Example 113, the object product (57%) was obtained.

¹H NMR (300 MHz, DMSO-d₆) δ ppm 2.34 (s, 2H) 2.39 (s, 2H) 5.46 (s, 2H) 6.91 (s, 1H) 6.95-7.06 (m, 1H) 7.20-7.27 (m, 1H) 7.28-7.39 (m, 1H) 7.50 (dd, J=1.70, 7.91 Hz, 1H) 7.77 (dd, J=1.60, 4.99 Hz, 1H) 7.94 (s, 1H) 8.30 (d, J=0.75 Hz, 1H) 8.77 (d, J=5.65 Hz, 1H) 10.29 (s, 1H)

### Example 125

### 4-[(3-chlorophenoxy)methyl]-N-(4,6-dimethylpyridin-2-yl)pyridine-2-carboxamide

Using 4-[(3-chlorophenoxy)methyl]pyridine-2-carboxylic acid obtained in Example 120(2) and in the same manner as in Example 113, the object product (44%) was obtained.

¹H NMR (300 MHz, DMSO-d₆) δ ppm 2.33 (s, 3H) 2.39 (s, 3H) 5.38 (s, 2H) 6.90 (s, 1H) 6.98-7.10 (m, 2H) 7.17 (t, J=2.17 Hz, 1H) 7.35 (t, J=8.19 Hz, 1H) 7.75 (dd, J=1.60, 4.99 Hz, 1H) 7.93 (s, 1H) 8.23 (d, J=0.75 Hz, 1H) 8.74 (d, J=4.90 Hz, 1H) 10.27 (s, 1H)

### Example 126

### 4-[(2-chloro-4-fluorophenoxy)methyl]-N-(4,6-dimethylpyridin-2-yl)pyridine-2-carboxamide

Using 4-[(2-chloro-4-fluorophenoxy)methyl]pyridine-2-carboxylic acid obtained in Example 121(2) and in the same manner as in Example 113, the object product (18%) was obtained.

¹H NMR (300 MHz, DMSO-d₆) δ ppm 2.34 (s, 3H) 2.39 (s, 3H) 5.44 (s, 2H) 6.92 (s, 1H) 7.16-7.30 (m, 2H) 7.51 (dd, J=2.64, 8.29 Hz, 1H) 7.75 (dd, J=1.60, 4.99 Hz, 1H) 7.94 (s, 1H) 8.27-8.30 (m, 1H) 8.71-8.81 (m, 1H) 10.29 (s, 1H)

### Example 127

### N-(4,6-dimethylpyridin-2-yl)-4-[(2-fluorophenoxy)methyl]pyridine-2-carboxamide

Using 4-[(2-fluorophenoxy)methyl]pyridine-2-carboxylic acid obtained in Example 122(2) and in the same manner as in Example 113, the object product (54%) was obtained.

¹H NMR (300 MHz, DMSO-d₆) δ ppm 2.33 (s, 3H) 2.39 (s, 3H) 5.43 (s, 2H) 6.91 (s, 1H) 6.94-7.04 (m, 1H) 7.09-7.19 (m, 1H) 7.19-7.33 (m, 2H) 7.75 (dd, J=1.70, 4.90 Hz, 1H) 7.93 (s, 1H) 8.26 (d, J=0.94 Hz, 1H) 8.68-8.83 (m, 1H) 10.28 (s, 1H)

### Example 128

### N-(4,6-dimethylpyridin-2-yl)-4-[(3-fluorophenoxy)methyl]pyridine-2-carboxamide

Using 4-[(3-fluorophenoxy)methyl]pyridine-2-carboxylic acid obtained in Example 123(2) and in the same manner as in Example 113, the object product (50%) was obtained.

¹H NMR (300 MHz, DMSO-d₆) δ ppm 2.33 (s, 3H) 2.39 (s, 3H) 5.37 (s, 2H) 6.77-6.87 (m, 1H) 6.89-7.00 (m, 3H) 7.29-7.41 (m, 1H) 7.75 (dd, J=1.70, 4.90 Hz, 1H) 7.93 (s, 1H) 8.21-8.25 (m, 1H) 8.66-8.82 (m, 1H) 10.28 (s, 1H)

### Example 129

### 4-[(2-chlorophenoxy)methyl]-N-[1-(4-fluorobenzyl)-1H-pyrazol-4-yl]pyridine-2-carboxamide dihydrochloride

Using 4-[(2-chlorophenoxy)methyl]pyridine-2-carboxylic acid obtained in Example 119(2) and in the same manner as in Example 114, the object product was obtained. The product was dissolved in ethyl acetate, hydrochloric acid (4N ethyl acetate solution, 5 mL) was added dropwise with stirring, and the mixture was stirred at room temperature for 1 hr. The resulting precipitate was collected by filtration to give the object product (51%) as a dihydrochloride.

¹H NMR (300 MHz, DMSO-d₆) δ ppm 5.31 (s, 2H) 5.43 (s, 2H) 6.93-7.05 (m, 1H) 7.11-7.25 (m, 3H) 7.26-7.37 (m, 3H) 7.49 (dd, J=1.51, 7.91 Hz, 1H) 7.70 (dd, J=1.41, 4.99 Hz, 1H) 7.79 (s, 1H) 8.19-8.28 (m, 2H) 8.73 (d, J=4.90 Hz, 1H) 11.00 (s, 1H)

### Example 130

### 4-[(3-chlorophenoxy)methyl]-N-[1-(4-fluorobenzyl)-1H-pyrazol-4-yl]pyridine-2-carboxamide

Using 4-[(3-chlorophenoxy)methyl]pyridine-2-carboxylic acid obtained in Example 120(2) and in the same manner as in Example 114, the object product (45%) was obtained.

¹H NMR (300 MHz, DMSO-d₆) δ ppm 5.32 (s, 2H) 5.36 (s, 2H) 6.97-7.10 (m, 2H) 7.11-7.24 (m, 3H) 7.26-7.41 (m, 3H) 7.68 (dd, J=1.60, 4.99 Hz, 1H) 7.81 (s, 1H) 8.16 (d, J=0.75 Hz, 1H) 8.24 (s, 1H) 8.72 (d, J=5.46 Hz, 1H) 10.99 (s, 1H)

### Example 131

### 4-[(2-chloro-4-fluorophenoxy)methyl]-N-[1-(4-fluorobenzyl)-1H-pyrazol-4-yl]pyridine-2-carboxamide

Using 4-[(2-chloro-4-fluorophenoxy)methyl]pyridine-2-carboxylic acid obtained in Example 121(2) and in the same manner as in Example 114, the object product (61%) was obtained.

¹H NMR (300 MHz, DMSO-d₆) δ ppm 5.32 (s, 2H) 5.41 (s, 2H) 7.12-7.38 (m, 6H) 7.51 (dd, J=2.73, 8.19 Hz, 1H) 7.68 (dd, J=1.60, 4.99 Hz, 1H) 7.80 (s, 1H) 8.20 (s, 1H) 8.25 (s, 1H) 8.73 (d, J=4.90 Hz, 1H) 10.99 (s, 1H)

### Example 132

### N-[1-(4-fluorobenzyl)-1H-pyrazol-4-yl]-4-[(2-fluorophenoxy)methyl]pyridine-2-carboxamide

Using 4-[(2-fluorophenoxy)methyl]pyridine-2-carboxylic acid obtained in Example 122(2) and in the same manner as in Example 114, the object product (75%) was obtained.

¹H NMR (300 MHz, DMSO-d₆) δ ppm 5.31 (s, 2H) 5.40 (s, 2H) 6.94-7.04 (m, 1H) 7.09-7.35 (m, 7H) 7.68 (dd, J=1.70, 5.09 Hz, 1H) 7.79 (s, 1H) 8.17 (d, J=0.75 Hz, 1H) 8.23 (s, 1H) 8.72 (d, J=5.65 Hz, 1H) 10.98 (s, 1H)

### Example 133

### N-[1-(4-fluorobenzyl)-1H-pyrazol-4-yl]-4-[(3-fluorophenoxy)methyl]pyridine-2-carboxamide

Using 4-[(3-fluorophenoxy)methyl]pyridine-2-carboxylic acid obtained in Example 123(2) and in the same manner as in Example 114, the object product (50%) was obtained.

¹H NMR (300 MHz, DMSO-d₆) δ ppm 5.32 (s, 2H) 5.35 (s, 2H) 6.77-6.86 (m, 1H) 6.88-6.99 (m, 2H) 7.14-7.22 (m, 2H) 7.27-7.40 (m, 3H) 7.68 (dd, J=1.60, 4.99 Hz, 1H) 7.80 (s, 1H) 8.16 (d, J=0.94 Hz, 1H) 8.23 (s, 1H) 8.71 (d, J=5.46 Hz, 1H) 10.99 (s, 1H)

### Example 134

### N-(1-benzyl-1H-pyrazol-4-yl)-4-[(2-chlorophenoxy)methyl]pyridine-2-carboxamide

### (1a) tert-butyl 4-amino-1H-pyrazole-1-carboxylate

4-Nitro-1H-pyrazole (20 g), triethylamine (27 mL) and di-tert-butyl dicarbonate (42.4 g) were dissolved in acetonitrile (200 mL) solution, and the mixture was stirred at room temperature for 3 days. The solvent was evaporated under reduced pressure, and the residue was washed with water and hexane to give tert-butyl 4-nitro-1H-pyrazole-1-carboxylate as a yellow solid (32.76 g). The compound was dissolved in methanol (250 mL), palladium carbon (6.6 g) was added, and the mixture was stirred at room temperature for 2 days under a hydrogen atmosphere. The catalyst was filtered through celite, and the filtrate was concentrated to give tert-butyl 4-amino-1H-pyrazole-1-carboxylate (19.5 g, 70%).

¹H NMR (300 MHz, DMSO-d₆) δ ppm 1.53 (s, 9H) 4.39 (s, 2H) 7.34 (d, J=4.92 Hz, 2H)

### (1) tert-butyl 4-[({4-[(2-chlorophenoxy)methyl]pyridin-2-yl}carbonyl)amino]-1H-pyrazole-1-carboxylate

To a solution of 4-[(2-chlorophenoxy)methyl]pyridine-2-carboxylic acid (2.64 g) obtained in Example 119(2) in tetrahydrofuran (50 mL) was added oxalyl chloride (1 mL) at room temperature, and then N,N-dimethylformamide (1 drop) was added. The mixture was stirred at room temperature for 40 min and tetrahydrofuran was evaporated under reduced pressure. The residue was dissolved in N,N-dimethylacetamide (50 mL). This solution was added dropwise to a solution of tert-butyl 4-amino-1H-pyrazole-1-carboxylate (2.01 g) synthesized in the above-mentioned (1a) in N,N-dimethylacetamide (50 mL), which was placed in advance in a different pear shape flask. The mixture was stirred at room temperature for 3 hr. Water was added, and the precipitated solid was collected by filtration and washed with water and hexane to give a gray solid (3.73 g, 87%).

¹H NMR (300 MHz, DMSO-d₆) δ ppm 1.60 (s, 9H) 5.45 (s, 2H) 6.97-7.06 (m, 1H) 7.19-7.25 (m, 1H) 7.28-7.37 (m, 1H) 7.50 (dd, J=1.51, 7.91 Hz, 1H) 7.73 (dd, J=1.60, 4.99 Hz, 1H) 8.15 (s, 1H) 8.24 (d, J=0.75 Hz, 1H) 8.58 (s, 1H) 8.77 (d, J=4.90 Hz, 1H) 11.28 (s, 1H)

### (2) 4-[(2-chlorophenoxy)methyl]-N-1H-pyrazol-4-ylpyridine-2-carboxamide

To a solution of tert-butyl 4-[({4-[(2-chlorophenoxy)methyl]pyridin-2-yl}carbonyl)amino]-1H-pyrazole-1-carboxylate (3.0 g) obtained in the above-mentioned reaction in ethyl acetate (50 mL) was added dropwise a solution of 4N hydrochloric acid ethyl acetate (20 mL) under ice-cooling. The reaction mixture was stirred at room temperature for 8 hr. The solvent was evaporated under reduced pressure, and the residue was suspended in water. The suspension was neutralized with saturated aqueous sodium hydrogen carbonate solution. The precipitated solid was collected by filtration, and washed with water and hexane to give the object product (2.02 g, 88%).

¹H NMR (300 MHz, DMSO-d₆) δ ppm 5.44 (s, 2H) 6.93-7.09 (m, 1H) 7.13-7.26 (m, 1H) 7.28-7.38 (m, 1H) 7.50 (d, J=6.97 Hz, 1H) 7.69 (d, J=4.14 Hz, 1H) 7.97 (d, J=7.77 Hz, 2H) 8.22 (s, 1H) 8.74 (d, J=4.71 Hz, 1H) 10.92 (s, 1H) 12.64 (brs, 1H)

### (3) N-(1-benzyl-1H-pyrazol-4-yl)-4-[(2-chlorophenoxy)methyl]pyridine-2-carboxamide

To a solution (1 mL) of 4-[(2-chlorophenoxy)methyl]-N-1H-pyrazol-4-ylpyridine-2-carboxamide (40 mg) obtained in the above-mentioned reaction in N,N-dimethylformamide were added potassium carbonate (55 mg) and benzyl bromide (34 mg). The mixture was stirred at 60° C. for 1 day, allowed to cool to room temperature, and water was added. The precipitated solid was collected by filtration, and washed with water and hexane to give the object product (35 mg, 83%).

¹H NMR (300 MHz, DMSO-d₆) δ ppm 5.32 (s, 2H) 5.43 (s, 2H) 6.95-7.06 (m, 1H) 7.17-7.40 (m, 7H) 7.49 (dd, J=1.51, 7.91 Hz, 1H) 7.65-7.73 (m, 1H) 7.79 (s, 1H) 8.22 (s, 2H) 8.73 (d, J=4.90 Hz, 1H) 10.98 (s, 1H)

### Example 135

### 4-[(2-chlorophenoxy)methyl]-N-[1-(4-fluorobutyl)-1H-pyrazol-4-yl]pyridine-2-carboxamide

To a solution (2 mL) of 4-[(2-chlorophenoxy)methyl]-N-1H-pyrazol-4-ylpyridine-2-carboxamide (65 mg) obtained in Example 134(2) in N,N-dimethylformamide was added sodium hydride (60%, 24 mg), and the mixture was stirred at room temperature for 10 min. Then, 1-bromo-4-fluorobutane (46 mg) was added, and the mixture was stirred at room temperature overnight. Ethyl acetate was added to the reaction mixture, and the mixture was washed with water. The organic layer was dehydrated by passing through PhaseSep, and the solvent was evaporated. The residue was purified by silica gel column (50% ethyl acetate/hexane) to give the object product (36.2 mg).

ESI(pos) 403 [M+H]⁺

### Example 136

### 4-[(2-chlorophenoxy)methyl]-N-{1-[3-(1H-pyrrol-1-yl)propyl]-1H-pyrazol-4-yl}pyridine-2-carboxamide

By operation in the same manner as in Example 135 and using the compound obtained in Example 134(2) as a starting material, the object product was obtained.

ESI(pos) 422 [M+H]⁺

### Example 137

### 4-[(2-chlorophenoxy)methyl]-N-[1-(pyridin-3-ylmethyl)-1H-pyrazol-4-yl]pyridine-2-carboxamide

By operation in the same manner as in Example 135 and using the compound obtained in Example 134(2) as a starting material, the object product was obtained.

ESI(pos) 420 [M+H]⁺

### Example 138

### N-[1-(4-chlorobenzyl)-1H-pyrazol-4-yl]-4-[(2-chlorophenoxy)methyl]pyridine-2-carboxamide

By operation in the same manner as in Example 135 and using the compound obtained in Example 134(2) as a starting material, the object product was obtained.

ESI(pos) 453 [M+H]⁺

### Example 139

### 4-[(2-chlorophenoxy)methyl]-N-[1-(3-fluorobenzyl)-1H-pyrazol-4-yl]pyridine-2-carboxamide

By operation in the same manner as in Example 135 and using the compound obtained in Example 134(2) as a starting material, the object product was obtained.

ESI(pos) 437 [M+H]⁺

### Example 140

### 4-[(2-chlorophenoxy)methyl]-N-[1-(2-fluorobenzyl)-1H-pyrazol-4-yl]pyridine-2-carboxamide

By operation in the same manner as in Example 135 and using the compound obtained in Example 134(2) as a starting material, the object product was obtained.

ESI(pos) 437 [M+H]⁺

### Example 141

### 4-[(2-chlorophenoxy)methyl]-N-[1-(2,2,2-trifluoroethyl)-1H-pyrazol-4-yl]pyridine-2-carboxamide trifluoroacetate

By operation in the same manner as in Example 135 and using the compound obtained in Example 134(2) as a starting material, the object product was obtained.

ESI(pos) 411 [M+H]⁺

### Example 142

### 4-[(2-chlorophenoxy)methyl]-N-[1-(2-hydroxybutyl)-1H-pyrazol-4-yl]pyridine-2-carboxamide trifluoroacetate

By operation in the same manner as in Example 135 and using the compound obtained in Example 134(2) as a starting material, the object product was obtained.

ESI(pos) 401 [M+H]⁺

### Example 143

### N-[1-(4-fluorobenzyl)-1H-pyrazol-4-yl]-3-(phenylsulfonyl)pyrrolidine-1-carboxamide

To a solution of phenyl [1-(4-fluorobenzyl)-1H-pyrazol-4-yl]carbamate (155 mg) in N,N-dimethylformamide (3 mL) were added 3-(phenylsulfonyl)pyrrolidine (105 mg) and triethylamine (0.2 mL) and the mixture was stirred at 80° C. overnight. The reaction mixture was allowed to cool to room temperature, extracted with ethyl acetate and washed with 5% aqueous sodium hydrogen carbonate solution. The organic layer was passed through PhaseSep, and the solvent was evaporated. The residue was purified by preparative HPLC. The fraction was neutralized with saturated aqueous sodium hydrogen carbonate solution, and the mixture was extracted with ethyl acetate. The organic layer was concentrated to give the object product (110 mg, 26%).

¹H NMR (300 MHz, CDCl₃) δ ppm 2.24-2.38 (m, 1H) 2.49-2.66 (m, 1H) 3.36-3.94 (m, 5H) 5.14-5.21 (m, 2H) 5.99 (s, 1H) 6.91-7.07 (m, 2H) 7.14-7.24 (m, 2H) 7.37 (s, 1H) 7.53-7.73 (m, 4H) 7.88-7.96 (m, 2H)

### Example 144

### 3-(2-chlorobenzyl)-N-[1-(4-fluorobenzyl)-1H-pyrazol-4-yl]pyrrolidine-1-carboxamide

By operation in the same manner as in Example 143, the object compound was obtained.

ESI(pos) 413 [M+H]⁺

### Example 145

### N-[1-(4-fluorobenzyl)-1H-pyrazol-4-yl]-3-(2-phenylethyl)pyrrolidine-1-carboxamide

By operation in the same manner as in Example 143, the object compound was obtained.

ESI(pos) 393 [M+H]⁺

### Example 146

### 4-[(3-chlorophenoxy)methyl]-N-[1-(4-fluorobutyl)-1H-pyrazol-4-yl]pyridine-2-carboxamide dihydrochloride

### (1) 1-(4-fluorobutyl)-4-nitro-1H-pyrazole

A suspension of 4-nitro-1H-pyrazole (3.39 g), 1-bromo-4-fluorobutane (5.09 g) and potassium carbonate (6.9 g) in N,N-dimethylformamide (100 mL) was stirred at 60° C. for 5 hr. After cooling to room temperature, ethyl acetate was added, and the mixture was washed 3 times with saturated aqueous sodium hydrogen carbonate solution. The organic layer was dried over sodium sulfate and concentrated under reduced pressure. The residue was purified by silica gel chromatography to give the object product (4.97 g, 89%).

¹H NMR (300 MHz, CDCl₃) δ ppm 1.62-1.82 (m, 2H) 2.02-2.14 (m, 2H) 4.23 (t, J=7.06 Hz, 2H) 4.42 (t, J=5.65 Hz, 1H) 4.58 (t, J=5.65 Hz, 1H) 8.08 (s, 1H) 8.15 (s, 1H)

### (2) 1-(4-fluorobutyl)-1H-pyrazol-4-amine

A suspension of 1-(4-fluorobutyl)-4-nitro-1H-pyrazole (4.9 g) obtained in the above-mentioned reaction and 10% palladium/carbon (50% containing water, 2 g) in methanol (150 mL) was stirred at room temperature overnight under a hydrogen atmosphere. After celite filtration, the filtrate was evaporated under reduced pressure to give the object product as a crude product. The obtained crude product was used for the next reaction without further purification.

### (3) 4-[(3-chlorophenoxy)methyl]-N-[1-(4-fluorobutyl)-1H-pyrazol-4-yl]pyridine-2-carboxamide dihydrochloride

Using 1-(4-fluorobutyl)-1H-pyrazol-4-amine obtained in the above-mentioned reaction and 4-[(3-chlorophenoxy)methyl]pyridine-2-carboxylic acid obtained in Example 120(2) and in the same manner as in Example 129, the object product (81%) was obtained as a dihydrochloride.

¹H NMR (300 MHz, DMSO-d₆) δ ppm 1.47-1.71 (m, 2H) 1.73-1.91 (m, 2H) 4.13 (t, J=6.82 Hz, 2H) 4.36 (t, J=6.06 Hz, 1H) 4.52 (t, J=6.06 Hz, 1H) 5.37 (s, 2H) 7.05 (d, J=8.33 Hz, 2H) 7.17 (t, J=2.27 Hz, 1H) 7.35 (t, J=8.14 Hz, 1H) 7.68 (dd, J=1.51, 4.92 Hz, 1H) 7.77 (s, 1H) 8.10-8.21 (m, 2H) 8.72 (d, J=4.92 Hz, 1H) 10.93 (s, 1H)

### Example 147

### 2-[(2-chlorophenoxy)methyl]-N-1H-pyrazol-4-ylmorpholine-4-carboxamide hydrochloride

### (1) tert-butyl 4-[(phenoxycarbonyl)amino]-1H-pyrazole-1-carboxylate

Using tert-butyl 4-amino-1H-pyrazole-1-carboxylate obtained in Example 134(1a) and in the same manner as in the synthesis of phenyl [4-(1H-pyrazol-1-ylmethyl)phenyl]carbamate shown in Example 15, the object product was obtained as a crude product. The obtained crude product was used for the next reaction without further purification.

### (2) tert-butyl 4-[({3-[(2-chlorophenoxy)methyl]piperidin-1-yl}carbonyl)amino]-1H-pyrazole-1-carboxylate

In the same manner as in Example 15 and using tert-butyl 4-[(phenoxycarbonyl)amino]-1H-pyrazole-1-carboxylate obtained in the above-mentioned reaction and 2-[(2-chlorophenoxy)methyl]morpholine hydrochloride obtained in Example 18(2), the object compound (81%) was obtained.

¹H NMR (300 MHz, DMSO-d₆) δ ppm 1.57 (s, 9H) 2.83-3.04 (m, 2H) 3.46-3.59 (m, 1H) 3.72-3.83 (m, 1H) 3.83-3.99 (m, 2H) 4.07-4.17 (m, 3H) 6.93-7.01 (m, 1H) 7.16-7.21 (m, 1H) 7.26-7.35 (m, 1H) 7.43 (dd, J=1.51, 7.95 Hz, 1H) 7.78 (s, 1H) 8.14 (s, 1H) 8.95 (s, 1H)

### (3) 2-[(2-chlorophenoxy)methyl]-N-1H-pyrazol-4-ylmorpholine-4-carboxamide hydrochloride

Using tert-butyl 4-[({3-[(2-chlorophenoxy)methyl]piperidin-1-yl}carbonyl)amino]-1H-pyrazole-1-carboxylate obtained in the above-mentioned reaction and in the same manner as in the synthesis of 3-[(2-chlorophenoxy)methyl]piperidine hydrochloride shown in Example 15, the object product (75%) was obtained as a hydrochloride.

¹H NMR (300 MHz, DMSO-d₆) δ ppm 2.79-3.00 (m, 2H) 3.45-3.57 (m, 1H) 3.72-3.81 (m, 1H) 3.92 (d, J=11.11 Hz, 2H) 4.10-4.19 (m, 3H) 6.93-7.01 (m, 1H) 7.15-7.22 (m, 1H) 7.27-7.34 (m, 1H) 7.43 (dd, J=1.60, 7.82 Hz, 1H) 7.79 (s, 2H) 8.90 (s, 1H)

### Example 148

### N-[1-(4-fluorobenzyl)-1H-pyrazol-4-yl]-2-[(2-fluorophenoxy)methyl]morpholine-4-carboxamide

### (1) 2-[(2-fluorophenoxy)methyl]morpholine hydrochloride

Using (4-benzylmorpholin-2-yl)methanol (1.04 g) and 2-fluorophenol (0.445 mL) and in the same manner as in the synthesis of N-tert-butoxycarbonyl-3-[(2-chlorophenoxy)methyl]piperidine shown in Example 15, 4-benzyl-2-[(2-fluorophenoxy)methyl]morpholine was obtained as a crude product. A suspension of this crude product (900 mg) and 5% palladium hydroxide/carbon (100 mg) in methanol (30 mL) was stirred overnight under a hydrogen atmosphere at room temperature. The reaction mixture was filtered through celite, and the solvent of the filtrate was evaporated under reduced pressure. The residue was dissolved in ethyl acetate, hydrochloric acid (4N ethyl acetate solution, 5 mL) was added dropwise with stirring, and the mixture was stirred at room temperature for 4 hr. The precipitate was collected by filtration and dried under reduced pressure to give the object product as a crude product. The obtained crude product was used for the next reaction without further purification.

### (2) N-[1-(4-fluorobenzyl)-1H-pyrazol-4-yl]-2-[(2-fluorophenoxy)methyl]morpholine-4-carboxamide

Using 2-[(2-fluorophenoxy)methyl]morpholine hydrochloride obtained in the above-mentioned reaction and in the same manner as in Example 16, the object product (81%) was obtained.

¹H NMR (300 MHz, CDCl₃) δ ppm 2.71-3.07 (m, 2H) 3.42-3.57 (m, 1H) 3.69-3.91 (m, 3H) 3.91-4.15 (m, 3H) 5.08 (s, 2H) 6.82-7.21 (m, 8H) 7.34 (s, 1H) 7.57 (s, 1H) 7.68 (s, 1H)

### Example 149

### N-[1-(4-fluorobenzyl)-1H-pyrazol-4-yl]-3-(phenoxymethyl)benzamide

In the same manner as in Example 3 and using phenol, the object compound (83%) was obtained.

¹H NMR (300 MHz, DMSO-d₆) δ ppm 5.18 (s, 2H) 5.31 (s, 2H) 6.90-6.99 (m, 1H) 7.00-7.07 (m, 2H) 7.12-7.23 (m, 2H) 7.25-7.36 (m, 4H) 7.54 (t, J=7.72 Hz, 1H) 7.60-7.68 (m, 2H) 7.85-7.95 (m, 1H) 8.02 (s, 1H) 8.17 (d, J=0.75 Hz, 1H) 10.51 (s, 1H)

### Example 150

### 3-[(2-chlorophenoxy)methyl]-N-1H-pyrazol-4-ylbenzamide

3-[(2-Chlorophenoxy)methyl]benzoic acid was suspended in tetrahydrofuran (50 mL), and oxalyl chloride (1.03 mL) and N,N-dimethylformamide (catalytic amount) were added. After stirring at room temperature for 30 min, the mixture was added dropwise to a suspension of 1H-pyrazol-4-amine (1.0 g) in N,N-dimethylacetamide (50 mL). After stirring at room temperature for 5 hr, ethyl acetate was added, and the mixture was washed 3 times with saturated aqueous sodium hydrogen carbonate solution. The organic layer was dried over sodium sulfate and concentrated under reduced pressure. The residue was purified by silica gel chromatography and recrystallized from ethyl acetate/hexane to give the object product (280 mg, 7.8%).

¹H NMR (300 MHz, DMSO-d₆) δ ppm 5.29 (s, 2H) 6.93-7.02 (m, 1H) 7.22-7.35 (m, 2H) 7.46 (dd, J=1.51, 7.91 Hz, 1H) 7.56 (t, J=7.72 Hz, 1H) 7.63-7.70 (m, 1H) 7.85 (brs, 2H) 7.89-7.95 (m, 1H) 8.02-8.06 (m, 1H) 10.47 (s, 1H) 12.63 (brs, 1H)

### Example 151

### 3-[(2-chlorophenoxy)methyl]-N-(1-methyl-1H-pyrazol-4-yl)benzamide

A suspension of 3-[(2-chlorophenoxy)methyl]-N-1H-pyrazol-4-ylbenzamide (230 mg) obtained in Example 150, methyl iodide (0.2 mL) and potassium carbonate (276 mg) in N,N-dimethylformamide (10 mL) was stirred at 60° C. overnight. Ethyl acetate was added, and the mixture was washed 3 times with saturated aqueous sodium hydrogen carbonate solution. The organic layer was dried over sodium sulfate and concentrated under reduced pressure. The residue was purified by silica gel chromatography to give the object product (73 mg, 31%).

¹H NMR (300 MHz, DMSO-d₆) δ ppm 3.82 (s, 3H) 5.29 (s, 2H) 6.93-7.03 (m, 1H) 7.21-7.36 (m, 2H) 7.42-7.48 (m, 1H) 7.51-7.61 (m, 2H) 7.63-7.70 (m, 1H) 7.88-7.95 (m, 1H) 8.00-8.06 (m, 2H) 10.46 (s, 1H)

### Example 152

### 3-[(2-chlorophenoxy)methyl]-N-[1-(cyclopropylmethyl)-1H-pyrazol-4-yl]benzamide

Using (bromomethyl)cyclopropane and in the same manner as in Example 151, the object product (40%) was obtained.

¹H NMR (300 MHz, DMSO-d₆) δ ppm 0.31-0.39 (m, 2H) 0.48-0.57 (m, 2H) 1.14-1.30 (m, 1H) 3.95 (d, J=7.19 Hz, 2H) 5.29 (s, 2H) 6.94-7.02 (m, 1H) 7.22-7.35 (m, 2H) 7.46 (dd, J=1.51, 7.95 Hz, 1H) 7.51-7.61 (m, 2H) 7.62-7.70 (m, 1H) 7.92 (d, J=7.57 Hz, 1H) 8.04 (s, 1H) 8.11 (s, 1H) 10.47 (s, 1H)

### Example 153

### 3-[(2-chlorophenoxy)methyl]-N-[1-(2-phenylethyl)-1H-pyrazol-4-yl]benzamide

Using (2-bromoethyl)benzene and in the same manner as in Example 151, the object product (78%) was obtained.

¹H NMR (300 MHz, DMSO-d₆) δ ppm 3.10 (t, J=7.38 Hz, 2H) 4.34 (t, J=7.38 Hz, 2H) 5.29 (s, 2H) 6.93-7.03 (m, 1H) 7.16-7.36 (m, 7H) 7.45 (dd, J=1.51, 7.95 Hz, 1H) 7.55 (t, J=7.76 Hz, 1H) 7.62 (s, 1H) 7.64-7.70 (m, 1H) 7.91 (d, J=7.95 Hz, 1H) 7.97-8.07 (m, 2H) 10.45 (s, 1H)

### Example 154

### 2-[(2-fluorophenoxy)methyl]-N-(6-methylpyridin-2-yl) morpholine-4-carboxamide

### (1) phenyl(6-methylpyridin-2-yl)carbamate

Using 6-methylpyridin-2-amine and in the same manner as in the synthesis of phenyl [4-(1H-pyrazol-1-ylmethyl)phenyl]carbamate shown in Example 15, the object product was obtained as a crude product. The obtained crude product was used for the next reaction without further purification.

### (2) 2-[(2-fluorophenoxy)methyl]-N-(6-methylpyridin-2-yl)morpholine-4-carboxamide

In the same manner as in Example 148 and using phenyl (6-methylpyridin-2-yl)carbamate obtained in the above-mentioned reaction, 2-[(2-fluorophenoxy)methyl]morpholine hydrochloride obtained in Example 148(1), the object compound (65%) was obtained.

¹H NMR (300 MHz, CDCl₃) δ ppm 2.39 (s, 3H) 2.95-3.22 (m, 2H) 3.55-3.68 (m, 1H) 3.79-4.20 (m, 6H) 6.78 (d, J=7.19 Hz, 1H) 6.84-7.12 (m, 4H) 7.46-7.56 (m, 1H) 7.69 (brs, 1H) 7.81 (d, J=7.95 Hz, 1H)

### Example 155

### 4-[(2-chloro-4-fluorophenoxy)methyl]-N-[1-(4-fluorobutyl)-1H-pyrazol-4-yl]pyridine-2-carboxamide

Using 4-[(2-chloro-4-fluorophenoxy)methyl]pyridine-2-carboxylic acid obtained in Example 121(2), 1-(4-fluorobutyl)-1H-pyrazol-4-amine obtained in Example 146(2) and in the same manner as in Example 114, the object product (77%) was obtained.

¹H NMR (300 MHz, DMSO-d₆) δ ppm 1.49-1.71 (m, 2H) 1.77-1.92 (m, 2H) 4.13 (t, J=6.82 Hz, 2H) 4.36 (t, J=6.06 Hz, 1 H) 4.52 (t, J=5.87 Hz, 1H) 5.41 (s, 2H) 7.16-7.29 (m, 2H) 7.50 (dd, J=2.65, 8.33 Hz, 1H) 7.68 (dd, J=1.51, 4.92 Hz, 1H) 7.76 (s, 1H) 8.15 (s, 1H) 8.20 (s, 1H) 8.73 (d, J=4.92 Hz, 1H) 10.93 (s, 1H)

### Example 156

### 4-[(2,5-dichlorophenoxy)methyl]-N-[1-(4-fluorobutyl)-1H-pyrazol-4-yl]pyridine-2-carboxamide

Using 4-[(2,5-dichlorophenoxy)methyl]pyridine-2-carboxylic acid obtained in Example 112(2) and in the same manner as in Example 155, the object product (86%) was obtained.

¹H NMR (300 MHz, DMSO-d₆) δ ppm 1.48-1.70 (m, 2H) 1.76-1.93 (m, 2H) 4.13 (t, J=6.82 Hz, 2H) 4.36 (t, J=6.06 Hz, 1H) 4.52 (t, J=6.06 Hz, 1H) 5.48 (s, 2H) 7.10 (dd, J=2.27, 8.33 Hz, 1H) 7.38 (d, J=2.27 Hz, 1H) 7.53 (d, J=8.71 Hz, 1H) 7.63-7.71 (m, 1H) 7.76 (s, 1H) 8.16 (s, 1H) 8.21 (s, 1H) 8.74 (d, J=5.30 Hz, 1H) 10.94 (s, 1H)

### Example 157

### N-[1-(4-fluorobutyl)-1H-pyrazol-4-yl]-4-[(2-fluorophenoxy)methyl]pyridine-2-carboxamide dihydrochloride

Using 4-[(2-fluorophenoxy)methyl]pyridine-2-carboxylic acid obtained in Example 122(2) and in the same manner as in Example 146, the object product (74%) was obtained as a dihydrochloride.

¹H NMR (300 MHz, DMSO-d₆) δ ppm 1.49-1.72 (m, 2H) 1.76-1.92 (m, 2H) 4.14 (t, J=6.82 Hz, 2H) 4.36 (t, J=6.06 Hz, 1H) 4.52 (t, J=6.06 Hz, 1H) 5.41 (s, 2H) 6.93-7.04 (m, 1H) 7.09-7.18 (m, 1H) 7.19-7.32 (m, 2H) 7.65-7.72 (m, 1H) 7.77 (s, 1H) 8.13-8.22 (m, 2H) 8.73 (d, J=4.92 Hz, 1H) 10.95 (s, 1H)

### Example 158

### N-[1-(4-fluorobutyl)-1H-pyrazol-4-yl]-4-[(3-fluorophenoxy)methyl]pyridine-2-carboxamide dihydrochloride

Using 4-[(3-fluorophenoxy)methyl]pyridine-2-carboxylic acid obtained in Example 123(2) and in the same manner as in Example 146, the object product (86%) was obtained as a dihydrochloride.

¹H NMR (300 MHz, DMSO-d₆) δ ppm 1.50-1.71 (m, 2H) 1.78-1.93 (m, 2H) 4.14 (t, J=6.82 Hz, 2H) 4.36 (t, J=6.06 Hz, 1H) 4.52 (t, J=5.87 Hz, 1H) 5.36 (s, 2H) 6.76-6.88 (m, 1H) 6.87-7.00 (m, 2H) 7.28-7.42 (m, 1H) 7.69 (dd, J=1.51, 4.92 Hz, 1H) 7.78 (s, 1H) 8.11-8.21 (m, 2H) 8.72 (d, J=5.30 Hz, 1H) 10.96 (s, 1H)

### Example 159

### 4-[(3-chlorophenoxy)methyl]-N-{1-[3-(1H-pyrrol-1-yl)propyl]-1H-pyrazol-4-yl}pyridine-2-carboxamide

### (1) 1-[3-(1H-pyrrol-1-yl)propyl]-1H-pyrazol-4-amine

Using 1-(3-bromopropyl)-1H-pyrrole and in the same manner as in the synthesis of 1-(4-fluorobutyl)-1H-pyrazol-4-amine shown in Example 146, the object product was obtained as a crude product. The obtained crude product was used for the next reaction without further purification.

### (2) 4-[(3-chlorophenoxy)methyl]-N-{1-[3-(1H-pyrrol-1-yl)propyl]-1H-pyrazol-4-yl}pyridine-2-carboxamide

Using 1-[3-(1H-pyrrol-1-yl)propyl]-1H-pyrazol-4-amine obtained in the above-mentioned reaction, 4-[(3-chlorophenoxy)methyl]pyridine-2-carboxylic acid obtained in Example 120(2) and in the same manner as in Example 114, the object product (81%) was obtained.

¹H NMR (300 MHz, DMSO-d₆) δ ppm 2.09-2.32 (m, 2H) 3.87 (t, J=6.82 Hz, 2H) 4.03 (t, J=6.82 Hz, 2H) 5.36 (s, 2H) 5.94-6.14 (m, 2H) 6.69-6.81 (m, 2H) 7.05 (d, J=9.09 Hz, 2H) 7.15-7.21 (m, 1H) 7.35 (t, J=8.14 Hz, 1H) 7.68 (d, J=4.92 Hz, 1H) 7.80 (s, 1H) 8.13 (s, 1H) 8.17 (s, 1H) 8.72 (d, J=4.92 Hz, 1H) 10.95 (s, 1H)

### Example 160

### 4-[(2-chloro-4-fluorophenoxy)methyl]-N-{1-[3-(1H-pyrrol-1-yl)propyl]-1H-pyrazol-4-yl}pyridine-2-carboxamide

Using 4-[(2-chloro-4-fluorophenoxy)methyl]pyridine-2-carboxylic acid obtained in Example 121(2) and in the same manner as in Example 159, the object product (61%) was obtained.

¹H NMR (300 MHz, DMSO-d₆) δ ppm 2.11-2.28 (m, 2H) 3.87 (t, J=6.88 Hz, 2H) 4.03 (t, J=6.88 Hz, 2H) 5.42 (s, 2H) 6.01 (t, J=2.07 Hz, 2H) 6.77 (t, J=2.07 Hz, 2H) 7.12-7.35 (m, 2H) 7.51 (dd, J=2.64, 8.29, Hz, 1H) 7.69 (dd, J=1.41, 4.99 Hz, 1H) 7.81 (s, 1H) 8.15 (s, 1H) 8.22 (s, 1H) 8.74 (d, J=4.90 Hz, 1H) 10.98 (s, 1H)

### Example 161

### 4-[(2,5-dichlorophenoxy)methyl]-N-{1-[3-(1H-pyrrol-1-yl)propyl]-1H-pyrazol-4-yl}pyridine-2-carboxamide

Using 4-[(2,5-dichlorophenoxy)methyl]pyridine-2-carboxylic acid obtained in Example 112(2) and in the same manner as in Example 159, the object product (66%) was obtained.

¹H NMR (300 MHz, DMSO-d₆) δ ppm 2.13-2.27 (m, 2H) 3.87 (t, J=6.88 Hz, 2H) 4.03 (t, J=6.88 Hz, 2H) 5.48 (s, 2H) 6.01 (t, J=2.07 Hz, 2H) 6.77 (t, J=2.07 Hz, 2H) 7.10 (dd, J=2.26, 8.48 Hz, 1H) 7.38 (d, J=2.45 Hz, 1H) 7.53 (d, J=8.48 Hz, 1H) 7.68 (dd, J=1.60, 4.99 Hz, 1H) 7.80 (s, 1H) 8.14 (s, 1H) 8.22 (s, 1H) 8.75 (d, J=5.65 Hz, 1H) 10.98 (s, 1H)

### Example 162

### 4-[(3-fluorophenoxy)methyl]-N-{1-[3-(1H-pyrrol-1-yl)propyl]-1H-pyrazol-4-yl}pyridine-2-carboxamide

Using 4-[(3-fluorophenoxy)methyl]pyridine-2-carboxylic acid obtained in Example 123(2) and in the same manner as in Example 159, the object product (72%) was obtained.

¹H NMR (300 MHz, DMSO-d₆) δ ppm 2.13-2.26 (m, 2H) 3.86 (t, J=6.82 Hz, 2H) 4.02 (t, J=6.82 Hz, 2H) 5.35 (s, 2H) 6.00 (t, J=2.08 Hz, 2H) 6.72-6.86 (m, 3H) 6.88-7.01 (m, 2H) 7.28-7.42 (m, 1H) 7.68 (dd, J=1.70, 5.11 Hz, 1H) 7.80 (s, 1H) 8.12 (s, 1H) 8.17 (s, 1H) 8.72 (d, J=4.92 Hz, 1H) 10.94 (s, 1H)

### Example 163

### 3-[(2-chlorophenoxy)methyl]-N-(1-pyridin-3-yl-1H-pyrazol-4-yl)benzamide

To a solution of 3-[(2-chlorophenoxy)methyl]-N-1H-pyrazol-4-ylbenzamide (80 mg) in N,N-dimethylformamide were added pyridin-3-ylboronic acid (60 mg), copper acetate (68 mg) and pyridine (0.04 mL) and the mixture was tightly sealed, subjected to microwave (Emrys Optimizer manufactured by Personal Chemistry) irradiation, and stirred at 150° C. for 4 min. The reaction solution was filtered through celite, and then the filtrate was extracted with ethyl acetate and washed with saturated aqueous sodium hydrogen carbonate solution. The organic layer was concentrated, and the residue was purified by preparative HPLC to give the object compound (4.4 mg, 4%). ESI(pos) 405[M+H]⁺

### Example 164

### 3-[(2-chlorophenoxy)methyl]-N-[1-(4-chlorophenyl)-1H-pyrazol-4-yl]benzamide

By operation in the same manner as in Example 163, the object product was obtained.

ESI(pos) 439[M+H]⁺

### Example 165

### 2-[(2-chlorophenoxy)methyl]-N-(6-methylpyridin-2-yl)morpholine-4-carboxamide

In the same manner as in Example 20 and using phenyl (6-methylpyridin-2-yl)carbamate obtained in Example 154(1), the object compound (77%) was obtained.

¹H NMR (300 MHz, DMSO-d₆) δ ppm 2.38 (s, 3H) 2.82-3.01 (m, 2H) 3.46-3.57 (m, 1H) 3.72-3.83 (m, 1H) 3.90 (dd, J=1.89, 11.36 Hz, 1H) 3.96-4.05 (m, 1H) 4.13 (d, J=4.54 Hz, 2H) 4.16-4.25 (m, 1H) 6.83 (d, J=6.82 Hz, 1H) 6.94-7.00 (m, 1H) 7.16-7.22 (m, 1H) 7.27-7.35 (m, 1H) 7.43 (dd, J=1.51, 7.95 Hz, 1H) 7.52-7.63 (m, 2H) 9.11 (s, 1H)

### Example 166

### 3-[2-(2-chlorophenoxy)ethyl]-N-[1-(4-fluorobenzyl)-1H-pyrazol-4-yl]piperidine-1-carboxamide

In the same manner as in Example 16 and using 3-[2-(2-chlorophenoxy)ethyl]piperidine hydrochloride obtained in Example 24(2), the object compound (53%) was obtained.

¹H NMR (300 MHz, CDCl₃) δ ppm 1.11-1.33 (m, 1H) 1.38-1.55 (m, 1H) 1.58-1.92 (m, 5H) 2.71-2.97 (m, 2H) 3.83-3.94 (m, 1H) 3.95-4.14 (m, 3H) 5.11 (s, 2H) 6.82-7.05 (m, 5H) 7.08-7.24 (m, 3H) 7.29-7.39 (m, 2H) 7.71 (s, 1H)

### Example 167

### 3-[(2,5-dichlorophenoxy)methyl]-N-[4-(1H-pyrazol-1-ylmethyl)phenyl]piperidine-1-carboxamide

### (1) N-tert-butoxycarbonyl-3-[(2,5-dichlorophenoxy)methyl]piperidine

Using N-tert-butoxycarbonyl-3-(hydroxymethyl)piperidine obtained in Example 15(1), 2,5-dichlorophenol and in the same manner as in the synthesis of N-tert-butoxycarbonyl-3-[(2-chlorophenoxy)methyl]piperidine shown in Example 15, the object product was obtained as a crude product. The obtained crude product was used for the next reaction without further purification.

### (2) 3-[(2,5-dichlorophenoxy)methyl]piperidine hydrochloride

Using N-tert-butoxycarbonyl-3-[(2,5-dichlorophenoxy)methyl]piperidine obtained in the above-mentioned reaction and in the same manner as in the synthesis of 3-[(2-chlorophenoxy)methyl]piperidine hydrochloride shown in Example 15, the object product was obtained as a crude product. The obtained crude product was used for the next reaction without further purification.

### (3) 3-[(2,5-dichlorophenoxy)methyl]-N-[4-(1H-pyrazol-1-ylmethyl)phenyl]piperidine-1-carboxamide

Using 3-[(2,5-dichlorophenoxy)methyl]piperidine hydrochloride obtained in the above-mentioned reaction and in the same manner as in Example 18, the object product (87%) was obtained.

¹H NMR (300 MHz, DMSO-d₆) δ ppm 1.28-1.54 (m, 2H) 1.65-1.76 (m, 1H) 1.82-2.02 (m, 2H) 2.75 (dd, J=10.17, 13.00 Hz, 1H) 2.82-2.94 (m, 1H) 3.90-4.03 (m, 3H) 4.10-4.20 (m, 1H) 5.22 (s, 2H) 6.24 (t, J=1.98 Hz, 1H) 7.01 (dd, J=2.26, 8.48 Hz, 1H) 7.10 (d, J=8.67 Hz, 2H) 7.26 (d, J=2.26 Hz, 1H) 7.35-7.47 (m, 4H) 7.75 (d, J=2.07 Hz, 1H) 8.50 (s, 1H)

### Example 168

### 3-[(2,5-dichlorophenoxy)methyl]-N-(4,6-dimethylpyridin-2-yl)piperidine-1-carboxamide

Using 3-[(2,5-dichlorophenoxy)methyl]piperidine hydrochloride obtained in Example 167(2) and in the same manner as in Example 17, the object product (70%) was obtained.

¹H NMR (300 MHz, DMSO-d₆) δ ppm 1.29-1.56 (m, 2H) 1.63-1.74 (m, 1H) 1.82-2.05 (m, 2H) 2.21 (s, 3H) 2.32 (s, 3H) 2.76-2.98 (m, 2H) 3.92-4.07 (m, 3H) 4.11-4.22 (m, 1H) 6.63 (s, 1H) 7.01 (dd, J=2.26, 8.48 Hz, 1H) 7.26 (d, J=2.45 Hz, 1H) 7.41-7.46 (m, 2H) 8.88 (s, 1H)

### Example 169

### 4-[(2-chlorophenoxy)methyl]-N-[1-(2-hydroxyethyl)-1H-pyrazol-4-yl]pyridine-2-carboxamide

A solution of 4-[(2-chlorophenoxy)methyl]-N-1H-pyrazol-4-ylpyridine-2-carboxamide (150 mg) obtained in Example 134(2), 2-bromoethanol (68 mg) and cesium carbonate (190 mg) in dimethylformamide (3 mL) was stirred at 90° C. for 24 hr. After cooling, aqueous ammonium chloride solution was added, and the mixture was extracted with ethyl acetate. The organic layer was washed with saturated aqueous solution and dried over sodium sulfate, and the solvent was evaporated under reduced pressure. The residue was purified by silica gel column (30% ethyl acetate/hexane to 40% ethyl acetate/hexane) to give the object product (42 mg) as a white solid.

¹H NMR (300 MHz, CDCl₃) δ ppm 3.03 (t, J=6.0 Hz, 1H) 4.00-4.05 (m, 2H) 4.24-4.27 (m, 2H) 5.25 (s, 2H) 6.91-6.98 (m, 2H) 7.19 (m, 1H) 7.41 (dd, J=1.8, 8.4 Hz, 1H) 7.64 (s, 1H) 7.69 (m, 1H) 8.16 (s, 1H) 8.28 (m, 1H) 8.61 (d, J=5.1 Hz, 1H) 9.82 (s, 1H)

### Example 170

### 4-[(2-chlorophenoxy)methyl]-N-[1-(4,4,4-trifluorobutyl)-1H-pyrazol-4-yl]pyridine-2-carboxamide dihydrochloride

Reactions similar to those in Example 169 were performed using 4-[(2-chlorophenoxy)methyl]-N-1H-pyrazol-4-ylpyridine-2-carboxamide (150 mg) obtained in Example 134(2), 4-bromo-1,1,1-trifluorobutane (105 mg) and cesium carbonate (190 mg). Then, reactions similar to those in Example 23(2) were performed to give the object product (80 mg) as a white solid.

¹H NMR (300 MHz, CDCl₃) δ ppm 2.02-2.22 (m, 4H) 4.23-4.38 (m, 2H) 5.35 (brs, 2H) 6.97-7.03 (m, 2H) 7.24 (m, 1H) 7.43 (d, J=7.5 Hz, 1H) 7.89 (brs, 1H) 7.98 (brs, 1H) 8.22 (brs, 1H) 8.62 (brs, 1H) 8.72 (brs, 1H) 10.9 (brs, 1H)

### Example 171

### 5-[(2-chlorophenoxy)methyl]-N-(4,6-dimethylpyridin-2-yl)-2-furamide

### (1) N-(4,6-dimethylpyridin-2-yl)-5-formyl-2-furamide

Reactions similar to those in Example 113 were performed using 5-formyl-2-furoic acid (4.0 g) and 4,6-dimethylpyridin-2-amine (3.5 g) to give the object product (1.7 g) as a white solid.

¹H NMR (300 MHz, CDCl₃) δ ppm 2.36 (s, 3H) 2.45 (s, 3H) 6.81 (s, 1H) 7.32-7.38 (m, 2H) 7.95 (s, 1H) 8.83 (brs, 1H) 9.76 (s, 1H)

### (2) N-(4,6-dimethylpyridin-2-yl)-5-(hydroxymethyl)-2-furamide

To a solution of N-(4,6-dimethylpyridin-2-yl)-5-formyl-2-furamide (350 mg) obtained in the above-mentioned reaction in methanol (5 mL) was added sodium borohydride (54 mg) under ice-cooling, and the mixture was stirred for 30 min. Water was added to the reaction solution, and the mixture was extracted with ethyl acetate. The organic layer was washed with saturated brine and dried over sodium sulfate, and the solvent was evaporated under reduced pressure. The residue was purified by silica gel column (30% ethyl acetate/hexane to 40% ethyl acetate/hexane) to give the object product (250 mg) as a white solid.

¹H NMR (300 MHz, DMSO-d₆) δ ppm 2.29 (s, 3H) 2.49 (s, 3H) 4.47 (d, J=5.7 Hz, 2H) 5.48 (t, J=5.7 Hz, 1H) 6.48 (d, J=3.3 Hz, 1H) 6.85 (s, 1H) 7.52 (d, J=3.3 Hz, 1H), 7.80 (s, 1H) 10.2 (s, 1H)

### (3) 5-[(2-chlorophenoxy)methyl]-N-(4,6-dimethylpyridin-2-yl)-2-furamide

Reactions similar to those in Example 15(2) were performed using N-(4,6-dimethylpyridin-2-yl)-5-(hydroxymethyl)-2-furamide (200 mg) obtained in the above-mentioned reaction and 2-chlorophenol (105 mg) to give the object product (120 mg) as a white solid.

¹H NMR (300 MHz, CDCl₃) δ ppm 2.33 (s, 3H) 2.42 (s, 3H) 5.08 (s, 2H) 6.59 (m, 1H) 6.75 (s, 1H) 6.93-7.00 (m, 2H) 7.19-7.22 (m, 2H) 7.37 (dd, J=1.5, 8.1 Hz, 1H) 7.96 (s, 1H) 8.74 (brs, 1H)

### Example 172

### 3-benzyl-1,2-dimethyl-N-[4-(1H-pyrazol-1-ylmethyl)phenyl]-1H-indole-6-carboxamide

### (1) ethyl 3-benzyl-2-methyl-1H-indole-6-carboxylate

A mixture of 3-hydrazinobenzoic acid hydrochloride (12.4 g), benzylacetone (11.8 mL), conc. sulfuric acid (4 mL) and ethanol (66 mL) was heated under reflux for 2 days. After cooling, the reaction mixture was poured into ice water, and the mixture was extracted with ether-ethyl acetate. The extract was washed successively with water, 5% aqueous sodium hydrogen carbonate solution, water and brine, and dried over magnesium sulfate. The solvent was evaporated under reduced pressure, petroleum ether/ether was added to the residue, and the precipitated solid was collected by filtration, washed with petroleum ether/ether and dried under reduced pressure to give the object compound (4.31 g, 25%).

¹H NMR (300 MHz, DMSO-d₆) δ ppm 1.32 (t, J=7.2 Hz, 3H) 2.41 (s, 3H) 4.01 (s, 2H) 4.28 (q, J=7.2 Hz, 2H) 7.00-7.25 (m, 5H) 7.40 (d, J=8.2 Hz, 1H) 7.52 (dd, J=1.8, 8.2 Hz, 1H), 7.89 (d, J=1.8 Hz, 1H) 11.22 (br, 1H)

### (2) ethyl 3-benzyl-1,2-dimethyl-1H-indole-6-carboxylate

To a solution of ethyl 3-benzyl-2-methyl-1H-indole-6-carboxylate (1.76 g) obtained in the above-mentioned reaction in N,N-dimethylformamide (30 mL) was added sodium hydride (60%, 288 mg) under ice-cooling, and the mixture was stirred at room temperature for 10 min. Then, methyl iodide (1.02 g) was added under ice-cooling, and the mixture was stirred at room temperature for 4 hr. Water was added to the reaction mixture, and the mixture was extracted with diethyl ether. The extract was washed with saturated brine, dried over magnesium sulfate and concentrated under reduced pressure. The residue was purified by silica gel column chromatography (2% ethyl acetate/hexane to 20% ethyl acetate/hexane) to give the object product (1.45 g, 79%) as a white powder.

¹H NMR (300 MHz, CDCl₃) δ ppm 1.41 (t, J=7.2 Hz, 3H) 2.39 (s, 3H) 3.74 (s, 3H) 4.10 (s, 2H) 4.39 (q, J=7.2 Hz, 2H) 7.10-7.26 (m, 5H) 7.41 (d, J=8.4 Hz, 1H) 7.73 (dd, J=1.5, 8.4 Hz, 1H) 8.03 (d, J=1.5 Hz, 1H)

### (3) 3-benzyl-1,2-dimethyl-1H-indole-6-carboxylic acid

A mixture of ethyl 3-benzyl-1,2-dimethyl-1H-indole-6-carboxylate (1.35 g, 4.39 mmol) obtained in the above-mentioned reaction, 1N aqueous sodium hydroxide solution (20 mL), tetrahydrofuran (10 mL) and ethanol (20 mL) was stirred at 80° C. for 5 hr. After allowing to cool in the air, the reaction mixture was neutralized with 1N aqueous hydrochloric acid solution. The precipitated powder was collected by filtration, washed with water and dried to give the object product (1.15 g, 94%) as a white powder.

¹H NMR (300 MHz, DMSO-d₆) δ ppm 2.44 (s, 3H) 3.73 (s, 3H) 4.05 (s, 2H) 7.10-7.26 (m, 5H), 7.44 (d, J=8.4 Hz, 1H), 7.56 (dd, J=1.5, 8.4 Hz, 1H), 7.99 (d, J=1.5 Hz, 1H)

### (4) 3-benzyl-1,2-dimethyl-N-[4-(1H-pyrazol-1-ylmethyl)phenyl]-1H-indole-6-carboxamide

Using 3-benzyl-1,2-dimethyl-1H-indole-6-carboxylic acid obtained in the above-mentioned reaction and in the same manner as in Example 1, the object product (44%) was obtained.

¹H NMR (300 MHz, DMSO-d₆) δ ppm 2.44 (s, 3H) 3.76 (s, 3H) 4.06 (s, 2H) 5.29 (s, 2H) 6.27 (t, J=2.08 Hz, 1H) 7.07-7.17 (m, 1H) 7.18-7.26 (m, 6H) 7.43-7.50 (m, 2H) 7.55-7.61 (m, 1H) 7.75 (d, J=8.33 Hz, 2H) 7.80 (d, J=2.27 Hz, 1H) 8.04 (s, 1H) 10.08 (s, 1H)

### Example 173

### 3-benzyl-N-(4,6-dimethylpyridin-2-yl)-1,2-dimethyl-1H-indole-6-carboxamide

A solution of 3-benzyl-1,2-dimethyl-1H-indole-6-carboxylic acid (196 mg) obtained in Example 172(3), 4,6-dimethylpyridin-2-amine (86 mg), 1-ethyl-3-(3-dimethylaminopropyl)carbodiimide hydrochloride (153 mg) and hydroxybenzotriazole hydrate (122 mg) in N-methylpyrrolidone (10 mL) was stirred at 100° C. for 10 min under microwave irradiation. Ethyl acetate (30 mL) was added, and the mixture was washed 3 times with saturated aqueous sodium hydrogen carbonate solution, and the organic layer was dried over sodium sulfate. The solvent was evaporated under reduced pressure, and the residue was purified by silica gel column chromatography to give the object product (42 mg, 16%).

¹H NMR (300 MHz, DMSO-d₆) δ ppm 2.31 (s, 3H) 2.41 (s, 3H) 2.44 (s, 3H) 3.78 (s, 3H) 4.05 (s, 2H) 6.84 (s, 1H) 7.07-7.17 (m, 1H) 7.18-7.25 (m, 4H) 7.44 (d, J=8.33 Hz, 1H) 7.63 (d, J=8.33 Hz, 1H) 7.92 (s, 1H) 8.28 (s, 1H) 10.37 (s, 1H)

### Example 174

### 1-benzyl-N-(4,6-dimethylpyridin-2-yl)-3-methyl-1H-thieno[2,3-c]pyrazole-5-carboxamide

Using 1-benzyl-3-methyl-1H-thieno[2,3-c]pyrazole-5-carboxylic acid and in the same manner as in Example 173, the object product (24%) was obtained.

¹H NMR (300 MHz, DMSO-d₆) δ ppm 2.27 (s, 3H) 2.39 (s, 6H) 5.40 (s, 2H) 6.84 (s, 1H) 7.29-7.48 (m, 5H) 7.75 (s, 1H) 8.32 (s, 1H) 10.69 (s, 1H)

### Example 175

### 3-(1H-benzimidazol-1-ylmethyl)-N-[4-(1H-pyrazol-1-ylmethyl)phenyl]benzamide

### (1) 3-(chloromethyl)-N-[4-(1H-pyrazol-1-ylmethyl)phenyl]benzamide

Using 3-(chloromethyl)benzoic acid and in the same manner as in Example 1, the object product was obtained as a crude product.

### (2) 3-(1H-benzimidazol-1-ylmethyl)-N-[4-(1H-pyrazol-1-ylmethyl)phenyl]benzamide

Using 3-(chloromethyl)-N-[4-(1H-pyrazol-1-ylmethyl)phenyl]benzamide (163 mg) obtained in the above-mentioned reaction and 1H-benzoimidazole (71 mg) and in the same manner as in Example 3, the object product (80%) was obtained.

¹H NMR (300 MHz, DMSO-d₆) δ ppm 5.29 (s, 2H) 5.59 (s, 2H) 6.26 (t, J=2.07 Hz, 1H) 7.17-7.25 (m, 4H) 7.44-7.56 (m, 4H) 7.63-7.73 (m, 3H) 7.78-7.81 (m, 1H) 7.83-7.89 (m, 1H) 7.89-7.92 (m, 1H) 8.45 (s, 1H) 10.28 (s, 1H)

### Example 176

### 3-(1H-indazol-1-ylmethyl)-N-[4-(1H-pyrazol-1-ylmethyl)phenyl]benzamide

Using 3-(chloromethyl)-N-[4-(1H-pyrazol-1-ylmethyl)phenyl]benzamide obtained in Example 175(1) and 1H-indazole and in the same manner as in Example 3, the object product (27%) was obtained.

¹H NMR (300 MHz, DMSO-d₆) δ ppm 5.29 (s, 2H) 5.75 (s, 2H) 6.26 (t, J=2.07 Hz, 1H) 7.10-7.24 (m, 3H) 7.33-7.49 (m, 4H) 7.65-7.89 (m, 7H) 8.13 (d, J=0.94 Hz, 1H) 10.27 (s, 1H)

### Example 177

### 3-(2H-indazol-2-ylmethyl)-N-[4-(1H-pyrazol-1-ylmethyl)phenyl]benzamide

The object product (18%) was obtained as a byproduct of Example 176.

¹H NMR (300 MHz, DMSO-d₆) δ ppm 5.29 (s, 2H) 5.73 (s, 2H) 6.22-6.31 (m, 1H) 6.97-7.11 (m, 1H) 7.16-7.28 (m, 3H) 7.43-7.55 (m, 3H) 7.59 (d, J=8.71 Hz, 1H) 7.66-7.95 (m, 6H) 8.53 (s, 1H) 10.30 (s, 1H)

### Example 178

### 4-(1H-pyrazol-1-ylmethyl)-N-pyridin-2-ylbenzamide

Using 4-(1H-pyrazol-1-ylmethyl)benzoic acid, pyridin-2-amine and in the same manner as in Example 173, the object product (20%) was obtained.

¹H NMR (300 MHz, DMSO-d₆) δ ppm 5.43 (s, 2H) 6.30 (t, J=2.08 Hz, 1H) 7.16 (dd, J=4.92, 7.19 Hz, 1H) 7.29 (d, J=8.33 Hz, 2H) 7.50 (s, 1H) 7.80-7.87 (m, 1H) 7.88 (d, J=2.27 Hz, 1H) 7.99 (d, J=7.95 Hz, 2H) 8.18 (d, J=8.33 Hz, 1H) 8.36-8.41 (m, 1H) 10.76 (s, 1H)

### Example 179

### 4-(1H-benzimidazol-1-ylmethyl)-N-pyrazin-2-ylbenzamide

Using 4-(1H-benzimidazol-1-ylmethyl)benzoic acid and pyrazin-2-amine and in the same manner as in Example 2, the object product (60%) was obtained.

¹H NMR (300 MHz, DMSO-d₆) δ ppm 5.62 (s, 2H) 7.15-7.27 (m, 2H) 7.43 (d, J=8.33 Hz, 2H) 7.48-7.56 (m, 1H) 7.62-7.71 (m, 1H) 8.01 (d, J=8.33 Hz, 2H) 8.41 (d, J=2.65 Hz, 1H) 8.44-8.48 (m, 2H) 9.39 (d, J=1.51 Hz, 1H) 11.08 (s, 1H)

### Example 180

### 4-(1H-benzimidazol-1-ylmethyl)-N-pyridin-2-ylbenzamide

Using 4-(1H-benzimidazol-1-ylmethyl)benzoic acid and pyridin-2-amine and in the same manner as in Example 173, the object product (54%) was obtained.

¹H NMR (300 MHz, DMSO-d₆) δ ppm 5.61 (s, 2H) 7.11-7.27 (m, 3H) 7.41 (d, J=8.33 Hz, 2H) 7.48-7.56 (m, 1H) 7.63-7.73 (m, 1H) 7.77-7.88 (m, 1H) 7.99 (d, J=8.33 Hz, 2H) 8.16 (d, J=8.33 Hz, 1H) 8.32-8.40 (m, 1H) 8.46 (s, 1H) 10.74 (s, 1H)

### Example 181

### 4-(1H-benzimidazol-1-ylmethyl)-N-(5-methylisoxazol-3-yl)benzamide

Using 4-(1H-benzimidazol-1-ylmethyl)benzoic acid and 5-methylisoxazol-3-amine and in the same manner as in Example 2, the object product (69%) was obtained.

¹H NMR (300 MHz, DMSO-d₆) δ ppm 2.40 (s, 3H) 5.60 (s, 2H) 6.73 (s, 1H) 7.14-7.26 (m, 2H) 7.41 (d, J=8.33 Hz, 2H) 7.46-7.55 (m, 1H) 7.61-7.75 (m, 1H) 7.96 (d, J=8.33 Hz, 2H) 8.44 (s, 1H) 11.25 (s, 1H)

### Example 182

### 3-[(2,3-dichlorophenoxy)methyl]-N-(4,6-dimethylpyridin-2-yl)benzamide trifluoroacetate

### (1) methyl 3-(hydroxymethyl)benzoate

To a solution of 3-(hydroxymethyl)benzoic acid (4.0 g) in methanol (100 mL) was added conc. sulfuric acid (0.28 mL). The reaction mixture was stirred under reflux for 2 hr. After cooling, saturated aqueous sodium hydrogen carbonate solution was added. Methanol was evaporated under reduced pressure, ethyl acetate was added to the residue, and the mixture was washed with saturated brine. The organic layer was dried over magnesium sulfate and the solvent was evaporated under reduced pressure to give a colorless oil. The object product was used for the next reaction without further purification.

### (2) methyl 3-[(tetrahydro-2H-pyran-2-yloxy)methyl]benzoate

To a solution of methyl 3-(hydroxymethyl)benzoate (3.0 g) obtained in the above-mentioned reaction and dihydropyran in acetonitrile was added p-toluenesulfonic acid monohydrate, and the mixture was stirred at room temperature for 1 day. Saturated aqueous sodium hydrogen carbonate solution was added, and acetonitrile was evaporated under reduced pressure. The mixture was extracted with ethyl acetate, and the organic layer was washed with saturated aqueous sodium hydrogen carbonate solution and saturated brine. The organic layer was dried over magnesium sulfate, and the solvent was evaporated under reduced pressure. The residue was purified by silica gel column (5% ethyl acetate/hexane to 20% ethyl acetate/hexane) to give a brown oil (5.0 g). The object product was used for the next reaction without further purification.

### (3) 3-[(tetrahydro-2H-pyran-2-yloxy)methyl]benzoic acid

To a mixed solution (3:1, 200 mL) of methyl 3-[(tetrahydro-2H-pyran-2-yloxy)methyl]benzoate (4.5 g) obtained in the above-mentioned reaction in methanol-water was added lithium hydroxide monohydrate (1.0 g) and the mixture was stirred at room temperature for 3 days. Citric acid was added and methanol was evaporated under reduced pressure. The mixture was extracted with ethyl acetate, and the organic layer was washed with saturated brine. The organic layer was dried over magnesium sulfate and the solvent was evaporated under reduced pressure. The residue was purified by silica gel column (5% ethyl acetate/hexane to 30% ethyl acetate/hexane) to give an oil (2.8 g).

¹H NMR (400 MHz, DMSO-d₆) δ ppm 1.38-1.60 (m, 4H) 1.61-1.82 (m, 2H) 3.44-3.53 (m, 1H) 3.74-3.84 (m, 1H) 4.52 (d, J=12.47 Hz, 1H) 4.70 (t, J=3.42 Hz, 1H) 4.74 (d, J=12.23 Hz, 1H) 7.49 (t, J=7.58 Hz, 1H) 7.59 (d, J=7.58 Hz, 1H) 7.87 (d, J=7.58 Hz, 1H) 7.93 (s, 1H) 13.01 (s, 1H)

### (4) N-(4,6-dimethylpyridin-2-yl)-3-[(tetrahydro-2H-pyran-2-yloxy)methyl]benzamide

3-[(Tetrahydro-2H-pyran-2-yloxy)methyl]benzoic acid (500 mg) obtained in the above-mentioned reaction was dissolved in tetrahydrofuran (20 mL) and N,N-dimethylformamide (5 mL). Thereto were added 4,6-dimethylaminopyridine (510 mg), 1-(3-dimethylaminopropyl)-3-ethylcarbodiimide hydrochloride (485 mg), 1-hydroxybenzotriazole (340 mg), triethylamine (0.35 mL) and N-methylimidazole (205 mg), and the mixture was stirred at room temperature for 4 hr. Tetrahydrofuran was evaporated under reduced pressure, ethyl acetate was added, and the mixture was washed with saturated aqueous sodium carbonate solution and saturated brine. The organic layer was dried over magnesium sulfate and the solvent was evaporated under reduced pressure. The residue was purified by silica gel column (10% ethyl acetate/hexane to 30% ethyl acetate/hexane) to give the object product as a colorless oil (390 mg). The object product was used for the next reaction without further purification.

### (5) N-(4,6-dimethylpyridin-2-yl)-3-(hydroxymethyl)benzamide

To a solution of N-(4,6-dimethylpyridin-2-yl)-3-[(tetrahydro-2H-pyran-2-yloxy)methyl]benzamide (390 mg) obtained in the above-mentioned reaction in methanol was added p-toluenesulfonic acid monohydrate (23 mg). The mixture was stirred at room temperature for 4 hr. Methanol was evaporated under reduced pressure. To the residue was added saturated aqueous sodium hydroxide solution, and the mixture was extracted with ethyl acetate. The organic layer was washed with saturated brine and dried over magnesium sulfate, and the solvent was evaporated under reduced pressure. The residue was purified by silica gel column (10% ethyl acetate/hexane to 30% ethyl acetate/hexane) to give a colorless oil. This was recrystallized from ethyl acetate/hexane to give a white solid (228 mg, 79%).

¹H NMR (400 MHz, DMSO-d₆) δ ppm 2.31 (s, 3H) 2.40 (s, 3H) 4.57 (d, J=5.62 Hz, 2H) 5.30 (t, J=5.75 Hz, 1H) 6.87 (s, 1H) 7.44 (t, J=7.58 Hz, 1H) 7.49-7.56 (m, 1H) 7.85-7.92 (m, 2H) 7.96 (s, 1H) 10.54 (s, 1H)

### (6) 3-[(2,3-dichlorophenoxy)methyl]-N-(4,6-dimethylpyridin-2-yl)benzamide trifluoroacetate

To a solution of N-(4,6-dimethylpyridin-2-yl)-3-(hydroxymethyl)benzamide (10 mg) obtained in the above-mentioned reaction in tetrahydrofuran (2 mL) were added triphenylphosphine polystyrene resin and 2,3-dichlorophenol (6.5 mg), and then di-tert-butyl azodicarboxylate (23 mg) was added. The mixture was stirred at 50° C. overnight, the solvent was evaporated, and the residue was purified by HPLC to give the object product at a purity of 100% (LCMS analysis).

ESI(pos) 401[M+H]⁺

### Example 183

### benzyl 4-{[(4,6-dimethylpyridin-2-yl)amino]carbonyl}piperidine-1-carboxylate

Using 1-[(benzyloxy)carbonyl]piperidine-4-carboxylic acid and in the same manner as in the synthesis of 5-butoxy-1-(2,4-dichlorobenzyl)-N-(4,6-dimethylpyridin-2-yl)-1H-pyrazole-3-carboxamide shown in Example 13, the object compound (25%) was obtained.

¹H NMR (300 MHz, DMSO-d₆) δ ppm 1.38-1.56 (m, 2H) 1.70-1.82 (m, 2H) 2.24 (s, 3H) 2.34 (s, 3H) 2.62-2.74 (m, 1H) 2.83 (brs, 2H) 4.04 (d, J=13.19 Hz, 2H) 5.08 (s, 2H) 6.79 (s, 1H) 7.18-7.48 (m, 5H) 7.75 (s, 1H) 10.37 (s, 1H)

### Example 184

### 4-[(imidazo[1,2-a]pyridin-8-yloxy)methyl]-N-pyridin-2-ylbenzamide

Using imidazo[1,2-a]pyridin-8-ol and in the same manner as in the synthesis of 4-{[2-(1,3,4-oxadiazol-2-yl)phenoxy]methyl}-N-pyridin-2-ylbenzamide shown in Example 107, the object product (20%) was obtained.

¹H NMR (300 MHz, DMSO-d₆) δ ppm 5.41 (s, 2H) 6.70-6.82 (m, 2H) 7.14-7.20 (m, 1H) 7.50 (d, J=1.13 Hz, 1H) 7.63 (d, J=8.48 Hz, 2H) 7.81-7.88 (m, 1H) 7.94 (d, J=1.13 Hz, 1H) 8.04-8.10 (m, 2H) 8.16-8.22 (m, 2H) 8.37-8.41 (m, 1H) 10.81 (s, 1H)

### Example 185

### ethyl 4-methyl-1-{4-[(pyridin-2-ylamino)carbonyl]benzyl}-1H-imidazole-5-carboxylate

Using ethyl 4-methyl-1H-imidazole-5-carboxylate and in the same manner as in the synthesis of 4-{[2-(1,3,4-oxadiazol-2-yl)phenoxy]methyl}-N-pyridin-2-ylbenzamide shown in Example 107, the object product (34%) was obtained.

¹H NMR (300 MHz, DMSO-d₆) δ ppm 1.19 (t, J=7.19 Hz, 3H) 2.39 (s, 3H) 4.15 (q, J=6.94 Hz, 2H) 5.56 (s, 2H) 7.11-7.22 (m, 3H) 7.79-7.88 (m, 1H) 7.98 (d, J=8.33 Hz, 2H) 8.05 (s, 1H) 8.17 (d, J=8.33 Hz, 1H) 8.38 (d, J=3.79 Hz, 1H) 10.75 (s, 1H)

### Example 186

### 4-[(2-oxo-1,3-benzoxazol-3(2H)-yl)methyl]-N-pyridin-2-ylbenzamide

Using 1,3-benzoxazol-2(3H)-one and in the same manner as in the synthesis of 4-{[2-(1,3,4-oxadiazol-2-yl)phenoxy]methyl}-N-pyridin-2-ylbenzamide shown in Example 107, the object product (59%) was obtained.

¹H NMR (300 MHz, DMSO-d₆) δ ppm 5.14 (s, 2H) 7.11-7.26 (m, 4H) 7.36-7.41 (m, 1H) 7.50 (d, J=8.29 Hz, 2H) 7.80-7.87 (m, 1H) 8.01 (d, J=8.48 Hz, 2H) 8.17 (d, J=8.48 Hz, 1H) 8.34-8.41 (m, 1H) 10.77 (s, 1H)

### Example 187

### N-(4,6-dimethylpyridin-2-yl)-4-[(3-oxo-2,3-dihydro-4H-1,4-benzoxazin-4-yl)methyl]benzamide

Using 4-[(3-oxo-2,3-dihydro-4H-1,4-benzoxazin-4-yl)methyl]benzoic acid and in the same manner as in the synthesis of 3-benzyl-N-(4,6-dimethylpyridin-2-yl)-1,2-dimethyl-1H-indole-6-carboxamide shown in Example 173, the object product (20%) was obtained.

¹H NMR (300 MHz, DMSO-d₆) δ ppm 2.29 (s, 3H) 2.39 (s, 3H) 4.84 (s, 2H) 5.23 (s, 2H) 6.86 (s, 1H) 6.90-7.07 (m, 4H) 7.39 (d, J=8.29 Hz, 2H) 7.86 (s, 1H) 7.98 (d, J=8.29 Hz, 2H) 10.58 (s, 1H)

### Example 188

### N-pyrazin-2-yl-4-{[2-(trifluoromethyl)-1H-benzimidazol-1-yl]methyl}benzamide

### (1) 4-(chloromethyl)-N-pyrazin-2-ylbenzamide

Using 4-(chloromethyl)benzoic acid and pyrazin-2-amine and in the same manner as in the synthesis of 3-[(2,5-dichlorophenoxy)methyl]-N-[1-(4-fluorobenzyl)-1H-pyrazol-4-yl]benzamide shown in Example 2, the object product (17%) was obtained.

¹H NMR (300 MHz, DMSO-d₆) δ ppm 4.85 (s, 2H) 7.59 (d, J=8.33 Hz, 2H) 8.05 (d, J=8.33 Hz, 2H) 8.43 (d, J=2.65 Hz, 1H) 8.47-8.52 (m, 1H) 9.43 (d, J=1.51 Hz, 1H) 11.16 (s, 1H) (2) N-pyrazin-2-yl-4-{[2-(trifluoromethyl)-1H-benzimidazol-1-yl]methyl}benzamide

Using 4-(chloromethyl)-N-pyrazin-2-ylbenzamide and 2-(trifluoromethyl)-1H-benzoimidazole obtained in the above-mentioned reaction and in the same manner as in the synthesis of N-[1-(4-fluorobenzyl)-1H-pyrazol-4-yl]-3-{[2-(trifluoromethyl)phenoxy]methyl}benzamide shown in Example 3, the object product (18%) was obtained.

¹H NMR (300 MHz, DMSO-d₆) δ ppm 5.86 (s, 2H) 7.25 (d, J=8.48 Hz, 2H) 7.40-7.53 (m, 2H) 7.70-7.75 (m, 1H) 7.88-7.97 (m, 1H) 8.05 (d, J=8.48 Hz, 2H) 8.43 (d, J=2.45 Hz, 1H) 8.48 (dd, J=1.51, 2.45 Hz, 1H) 9.43 (d, J=1.51 Hz, 1H) 11.12 (s, 1H)

### Example 189

### ethyl 5-methyl-1-{4-[(pyridin-2-ylamino)carbonyl]benzyl}-1H-imidazole-4-carboxylate

Using ethyl 4-methyl-1H-imidazole-5-carboxylate and in the same manner as in the synthesis of 4-{[2-(1,3,4-oxadiazol-2-yl)phenoxy]methyl}-N-pyridin-2-ylbenzamide shown in Example 107, the object product (20%) was obtained.

¹H NMR (300 MHz, DMSO-d₆) δ ppm 1.26 (t, J=7.06 Hz, 3H) 2.35 (s, 3H) 4.20 (q, J=7.10 Hz, 2H) 5.35 (s, 2H) 7.13-7.20 (m, 1H) 7.24 (d, J=8.48 Hz, 2H) 7.80-7.87 (m, 2H) 8.02 (d, J=8.48 Hz, 2H) 8.18 (d, J=8.29 Hz, 1H) 8.36-8.40 (m, 1H) 10.77 (s, 1H)

### Example 190

### 4-[(imidazo[1,2-a]pyridin-8-yloxy)methyl]-N-pyrazin-2-ylbenzamide

Using 4-(chloromethyl)-N-pyrazin-2-ylbenzamide obtained in Example 188(1) and imidazo[1,2-a]pyridin-8-ol and in the same manner as in the synthesis of N-[1-(4-fluorobenzyl)-1H-pyrazol-4-yl]-3-{[2-(trifluoromethyl)phenoxy]methyl}benzamide shown in Example 3, the object product (16%) was obtained.

¹H NMR (300 MHz, DMSO-d₆) δ ppm 5.42 (s, 2H) 6.69-6.82 (m, 2H) 7.50 (d, J=1.13 Hz, 1H) 7.65 (d, J=8.48 Hz, 2H) 7.94 (d, J=1.13 Hz, 1H) 8.10 (d, J=8.29 Hz, 2H) 8.18 (dd, J=1.13, 6.40 Hz, 1H) 8.42 (d, J=2.45 Hz, 1H) 8.49 (dd, J=1.51, 2.45 Hz, 1H) 9.43 (d, J=1.32 Hz, 1H) 11.15 (s, 1H)

### Example 191

### 4-(3H-imidazo[4,5-b]pyridin-3-ylmethyl)-N-pyridin-2-ylbenzamide

Using 3H-imidazo[4,5-b]pyridine and in the same manner as in the synthesis of 4-{[2-(1,3,4-oxadiazol-2-yl)phenoxy]methyl}-N-pyridin-2-ylbenzamide shown in Example 107, the object product (21%) was obtained.

¹H NMR (300 MHz, DMSO-d₆) δ ppm 5.61 (s, 2H) 7.15 (dd, J=4.92, 6.44 Hz, 1H) 7.31 (dd, J=4.73, 8.14 Hz, 1H) 7.43 (d, J=8.33 Hz, 2H) 7.79-7.86 (m, 1H) 7.98 (d, J=8.71 Hz, 2H) 8.09-8.14 (m, 1H) 8.17 (d, J=8.33 Hz, 1H) 8.34-8.40 (m, 2H) 8.66 (s, 1H) 10.73 (s, 1H)

### Example 192

### 3-(1H-benzimidazol-1-ylmethyl)-N-[1-(4-fluorobenzyl)-1H-pyrazol-4-yl]benzamide

Using 1H-benzoimidazole and in the same manner as in the synthesis of N-[1-(4-fluorobenzyl)-1H-pyrazol-4-yl]-3-{[2-(trifluoromethyl)phenoxy]methyl}benzamide shown in Example 3, the object product (76%) was obtained.

¹H NMR (300 MHz, DMSO-d₆) δ ppm 5.30 (s, 2H) 5.59 (s, 2H) 7.12-7.25 (m, 4H) 7.26-7.35 (m, 2H) 7.42-7.49 (m, 2H) 7.49-7.55 (m, 1H) 7.62-7.70 (m, 2H) 7.83-7.90 (m, 1H) 7.93 (s, 1H) 8.14 (s, 1H) 8.45 (s, 1H) 10.55 (brs, 1H)

### Example 193

### N-[4-(1H-pyrazol-1-ylmethyl)phenyl]-4-{[3-(trifluoromethyl)phenoxy]methyl}pyridine-2-carboxamide

Using 4-(1H-pyrazol-1-ylmethyl)aniline and 4-{[3-(trifluoromethyl)phenoxy]methyl}pyridine-2-carboxylic acid and in the same manner as in Example 113, the object product (40%) was obtained.

¹H NMR (300 MHz, DMSO-d₆) δ ppm 5.30 (s, 2H) 5.43 (s, 2H) 6.27 (t, J=2.08 Hz, 1H) 7.23 (d, J=8.33 Hz, 2H) 7.31-7.42 (m, 3H) 7.46 (s, 1H) 7.57 (t, J=7.76 Hz, 1H) 7.74 (dd, J=1.51, 4.92 Hz, 1H) 7.81 (d, J=1.89 Hz, 1H) 7.86 (d, J=8.33 Hz, 2H) 8.23 (s, 1H) 8.75 (d, J=5.30 Hz, 1H) 10.66 (s, 1H)

### Example 194

### N-[1-(4-fluorobenzyl)-1H-pyrazol-4-yl]-4-{[3-(trifluoromethyl)phenoxy]methyl}pyridine-2-carboxamide

Using 1-(4-fluorobenzyl)-1H-pyrazol-4-amine and 4-{[3-(trifluoromethyl)phenoxy]methyl}pyridine-2-carboxylic acid and in the same manner as in Example 113, the object product (74%) was obtained.

¹H NMR (300 MHz, DMSO-d₆) δ ppm 5.31 (s, 2H) 5.42 (s, 2H) 7.17 (t, J=8.90 Hz, 2H) 7.25-7.43 (m, 5H) 7.57 (t, J=7.95 Hz, 1H) 7.70 (d, J=4.92 Hz, 1H) 7.80 (s, 1H) 8.18 (s, 1H) 8.22 (s, 1H) 8.72 (d, J=4.92 Hz, 1H) 10.97 (s, 1H)

### Example 195

### N-(4,6-dimethylpyridin-2-yl)-4-{[3-(trifluoromethyl)phenoxy]methyl}pyridine-2-carboxamide

Using 4,6-dimethylpyridin-2-amine and 4-{[3-(trifluoromethyl)phenoxy]methyl}pyridine-2-carboxylic acid and in the same manner as in Example 113, the object product (58%) was obtained.

¹H NMR (300 MHz, DMSO-d₆) δ ppm 2.33 (s, 3H) 2.39 (s, 3H) 5.44 (s, 2H) 6.90 (s, 1H) 7.28-7.45 (m, 3H) 7.57 (t, J=7.95 Hz, 1H) 7.78 (d, J=4.54 Hz, 1H) 7.93 (s, 1H) 8.26 (s, 1H) 8.75 (d, J=4.92 Hz, 1H) 10.28 (s, 1H)

### Example 196

### 5-[(2-chlorophenoxy)methyl]-N-[1-(4-fluorobenzyl)-1H-pyrazol-4-yl]-2-furamide

Using 1-(4-fluorobenzyl)-1H-pyrazol-4-amine and 5-[(2-chlorophenoxy)methyl]-2-furoic acid and in the same manner as in Example 113, the object product (75%) was obtained.

¹H NMR (300 MHz, DMSO-d₆) δ ppm 5.20 (s, 2H) 5.28 (s, 2H) 6.75 (s, 1H) 6.98 (s, 1H) 7.11-7.23 (m, 3H) 7.24-7.36 (m, 4H) 7.43 (m, 1H) 7.61 (m, 1H) 8.05 (s, 1H) 10.38 (s, 1H)

### Example 197

### 4-[(2-chloro-5-fluorophenoxy)methyl]-N-[4-(1H-pyrazol-1-ylmethyl)phenyl]pyridine-2-carboxamide

Using 4-(1H-pyrazol-1-ylmethyl)aniline and 4-[(2-chloro-5-fluorophenoxy)methyl]pyridine-2-carboxylic acid and in the same manner as in Example 113, the object product (58%) was obtained.

¹H NMR (300 MHz, DMSO-d₆) δ ppm 5.30 (s, 2H) 5.46 (s, 2H) 6.26 (m, 1H) 6.89 (m, 1H) 7.20-7.24 (m, 3H) 7.45 (m, 1H) 7.53 (dd, J=6.3, 9.0 Hz, 1H) 7.71 (m, 1H) 7.80-7.87 (m, 3H) 8.23 (s, 1H) 8.76 (d, J=4.8 Hz, 1H) 10.7 (s, 1H)

### Example 198

### 4-[(2-chloro-5-fluorophenoxy)methyl]-N-[1-(4-fluorobenzyl)-1H-pyrazol-4-yl]pyridine-2-carboxamide

Using 1-(4-fluorobenzyl)-1H-pyrazol-4-amine and 4-[(2-chloro-5-fluorophenoxy)methyl]pyridine-2-carboxylic acid and in the same manner as in Example 113, the object product (60%) was obtained.

¹H NMR (300 MHz, DMSO-d₆) δ ppm 5.31 (s, 2H) 5.45 (s, 2H) 6.88 (m, 1H) 7.17-7.24 (m, 2H) 7.30-7.33 (m, 2H) 7.53 (dd, J=6.0, 9.0 Hz, 1H) 7.67 (dd, J=1.5, 4.8 Hz, 1H) 7.79 (s, 1H) 8.19 (s, 1H) 8.24 (s, 1H) 8.73 (d, J=5.1 Hz, 1H) 11.0 (s, 1H)

### Example 199

### 4-[(2-chloro-5-fluorophenoxy)methyl]-N-(4,6-dimethylpyridin-2-yl)pyridine-2-carboxamide

Using 4,6-dimethylpyridin-2-amine and 4-[(2-chloro-5-fluorophenoxy)methyl]pyridine-2-carboxylic acid and in the same manner as in Example 113, the object product (58%) was obtained.

¹H NMR (300 MHz, DMSO-d₆) δ ppm 2.33 (s, 3H) 2.39 (s, 3H) 5.47 (s, 2H) 6.85-6.91 (m, 2H) 7.21 (dd, J=2.7, 10.8 Hz, 1H) 7.54 (dd, J=6.0, 8.7 Hz, 1H) 7.74 (dd, J=1.2, 5.1 Hz, 1H) 7.94 (s, 1H) 8.28 (s, 1H) 8.76 (d, J=5.1 Hz, 1H) 10.3 (s, 1H)

### Example 200

### 4-[(2-chlorophenoxy)methyl]-N-[1-(3-hydroxypropyl)-1H-pyrazol-4-yl]pyridine-2-carboxamide

By reaction in the same manner as in Example 169 and using 4-[(2-chlorophenoxy)methyl]-N-1H-pyrazol-4-ylpyridine-2-carboxamide (200 mg) and 2-bromoethanol (101 mg), the object product (35 mg) was obtained as a white solid.

¹H NMR (300 MHz, DMSO-d₆) δ ppm 1.87-1.92 (m, 2H) 3.30-3.41 (m, 2H) 4.13 (t, J=6.9 Hz, 2H) 4.58 (t, J=6.9 Hz, 1H) 5.43 (s, 2H) 6.98 (m, 1H) 7.20 (m, 1H) 7.32 (m, 1H), 7.48 (dd, J=1.5, 7.8 Hz, 1H) 7.68 (dd, J=1.8, 5.1 Hz, 1H), 7.74 (s, 1H), 8.11 (s, 1H), 8.21 (s, 1H), 8.72 (d, J=5.1 Hz, 1H), 10.9 (brs, 1H)

### Example 201

### 3-{[(2-chlorophenyl)amino]methyl}-N-[1-(4-fluorobenzyl)-1H-pyrazol-4-yl]benzamide

### (1) ethyl 3-{[(2-chlorophenyl)amino]methyl}benzoate

A solution of ethyl 3-(bromomethyl)benzoate (3.8 g), 2-chloroaniline (2.3 g) and diisopropylamine (3.5 mL) in acetonitrile (40 mL) was stirred at room temperature for 2 hr. The solvent was evaporated under reduced pressure, and the residue was washed with diisopropyl ether to give the object product (3.5 g, 77%). The product was used for the next reaction without further purification.

### (2) 3-{[(2-chlorophenyl)amino]methyl}benzoic acid

To a mixed solution of ethyl 3-{[(2-chlorophenyl)amino]methyl}benzoate (0.53 g) obtained in the above-mentioned reaction in tetrahydrofuran and methanol (1:1, 10 mL) was added 2N lithium hydroxide (7 mL), and the mixture was stirred at room temperature for 2 hr. Water (20 mL) was added to the reaction solution, and the mixture was acidified with 1N aqueous hydrochloric acid solution. The obtained precipitate was collected by filtration, and dried under reduced pressure to give the object product (0.4 g, 84%).

### (3) 3-{[(2-chlorophenyl)amino]methyl}-N-[1-(4-fluorobenzyl)-1H-pyrazol-4-yl]benzamide

By reaction in the same manner as in Example 113 and using 3-{[(2-chlorophenyl)amino]methyl}benzoic acid (0.16 g) obtained in the above-mentioned reaction, 1-(4-fluorobenzyl)-1H-pyrazol-4-amine (0.11 g) and O-(benzotriazol-1-yl)-N,N,N,N-tetramethyluroniumtetrafluoroborate (0.19 mg), the object product (0.24 g, 90%) was obtained as a white solid.

¹H NMR (300 MHz, DMSO-d₆) δ ppm 4.49 (d, J=7.5 Hz, 2H) 5.31 (s, 2H) 6.05-6.12 (m, 1H) 6.52-6.60 (m, 2H) 7.02 (t, J=8.5 Hz, 1H) 7.12-7.21 (m, 2H) 7.25 (d, J=8.5 Hz, 1H) 7.28-7.35 (m, 2H) 7.42-7.48 (m, 1H) 7.49-7.55 (m, 1H) 7.62 (s, 1H) 7.78 (d, J=8.5 Hz, 1H) 7.91 (s, 1H) 8.12 (s, 1H), 10.37 (brs, 1H)

### Example 202

### 2-[(2-chloro-5-fluorophenoxy)methyl]-N-(6-ethylpyridin-2-yl) morpholine-4-carboxamide

### (1) phenyl (6-ethylpyridin-2-yl)carbamate

To a solution of 6-ethylpyridin-2-amine (1 g) in tetrahydrofuran (20 mL) was added phenyl chloride carbonate (1.2 mL) under ice-cooling, and the mixture was stirred at room temperature for 8 hr. Saturated aqueous sodium hydroxide solution was added to the residue, and the mixture was extracted with ethyl acetate. The organic layer was washed with saturated brine and dried over magnesium sulfate, and the solvent was evaporated under reduced pressure. The residue was washed with diisopropyl ether to give the object product (0.8 g) as a white solid.

¹H NMR (300 MHz, CDCl₃) δ ppm 1.28 (t, J=7.2 Hz, 3H) 2.73 (q, J=7.2 Hz, 2H) 6.89 (d, J=6.9 Hz, 1H) 7.09-7.42 (m, 5H) 7.61 (t, J=7.5 Hz, 1H) 7.76 (d, J=8.4 Hz, 1H) 7.89 (brs, 1H)

### (2) 2-[(2-chloro-5-fluorophenoxy)methyl]-N-(6-ethylpyridin-2-yl)morpholine-4-carboxamide

Phenyl (6-ethylpyridin-2-yl)carbamate (0.28 g) obtained in the above-mentioned reaction, 2-[(2-chloro-5-fluorophenoxy)methyl]morpholine hydrochloride (0.25 g) synthesized by a method similar to that in Example 18 and triethylamine (0.5 mL) were heated in dimethylformamide (5 mL) solution at 70° C. for 5 hr. Saturated aqueous sodium hydroxide solution was added to the residue, and the mixture was extracted with ethyl acetate. The organic layer was washed with saturated brine. The organic layer was dried over magnesium sulfate and the solvent was evaporated under reduced pressure. The residue was purified by silica gel column (20% ethyl acetate/hexane to 30% ethyl acetate/hexane) to give a pale-yellow oil (0.12 g).

¹H NMR (300 MHz, CDCl₃) δ ppm 1.25 (t, J=6.6 Hz, 3H) 2.67 (q, J=6.6 Hz, 2H) 3.07-3.23 (m, 2H) 3.65 (m, 1H) 3.90-3.94 (m, 2H) 4.01-4.20 (m, 2H) 6.62-6.72 (m, 2H) 6.81 (d, J=7.2 Hz, 1H) 7.19 (brs, 1H) 7.30 (dd, J=6.0, 8.4 Hz, 1H) 7.56 (t, J=7.2 Hz, 1H) 7.79 (d, J=7.8 Hz, 1H)

### Example 203

### N-[1-(pyridin-2-ylmethyl)-1H-pyrazol-4-yl]-4-{([3-(trifluoromethyl)phenoxy]methyl}pyridine-2-carboxamide

### (1) tert-butyl 4-{[(4-{[3-(trifluoromethyl)phenoxy]methyl}pyridin-2-yl)carbonyl]amino}-1H-pyrazole-1-carboxylate

4-{[3-(Trifluoromethyl)phenoxy]methyl}pyridine-2-carboxylic acid (500 mg), tert-butyl 4-amino-1H-pyrazole-1-carboxylate (310 mg), o-(benzotriazol-1-yl)-N,N,N,N-tetramethyluroniumtetrafluoroborate (592 mg) and triethylamine (0.32 mL) were dissolved in dimethylformamide (20 mL) solution, and the mixed solution was stirred at room temperature for 4 days. The reaction solution was added to water, and the mixture was extracted with ethyl acetate. The organic layer was washed with saturated brine and dried over magnesium sulfate. The desiccant was filtered off and the filtrate was concentrated. The residue was purified by silica gel chromatography (Si, 50% ethyl acetate/hexane) to give the object product (580 mg, 81%).

¹H NMR (300 MHz, CDCl₃) δ ppm 1.60 (s, 9H) 5.44 (s, 2H) 7.30-7.44 (m, 3H) 7.56 (d, J=7.72 Hz, 1H) 7.75 (dd, J=1.41, 4.99 Hz, 1H) 8.15 (s, 1H) 8.21 (s, 1H) 8.58 (s, 1H) 8.76 (d, J=4.90 Hz, 1H) 11.30 (s, 1H)

### (2) N-1H-pyrazol-4-yl-4-{[3-(trifluoromethyl)phenoxy]methyl}pyridine-2-carboxamide dihydrochloride

tert-Butyl 4-{[(4-{[3-(trifluoromethyl)phenoxy]methyl}pyridin-2-yl)carbonyl]amino}-1H-pyrazole-1-carboxylate (580 mg) obtained in the above-mentioned reaction was dissolved in 4N hydrochloric acid-ethyl acetate solution, and the mixture was stirred at room temperature overnight. The precipitate was collected by filtration and washed with hexane to quantitatively give the object product.

¹H NMR (300 MHz, DMSO-d₆) δ ppm 5.43 (s, 2H) 7.30-7.44 (m, 3 H) 7.57 (t, J=7.82 Hz, 1H) 7.71 (dd, J=1.60, 4.99 Hz, 1H) 7.85 (br. s., 1H) 8.09 (br. s., 1H) 8.20 (d, J=0.75 Hz, 1H) 8.73 (d, J=5.09 Hz, 1H) 10.93 (s, 1H)

### (3) N-[1-(pyridin-2-ylmethyl)-1H-pyrazol-4-yl]-4-{[3-(trifluoromethyl)phenoxy]methyl}pyridine-2-carboxamide

N-1H-Pyrazol-4-yl-4-{[3-(trifluoromethyl)phenoxy]methyl}pyridine-2-carboxamide dihydrochloride (100 mg) obtained in the above-mentioned reaction, 2-bromomethylpyridine hydrobromide (104 mg) and potassium carbonate (114 mg) were dissolved in dimethylformamide (5 mL) solution, and the mixed solution was stirred at 60° C. overnight. The reaction solution was poured into water, and the mixture was extracted with ethyl acetate. The organic layer was washed with saturated brine, dried over magnesium sulfate. The desiccant was filtered off and the filtrate was concentrated. The residue was purified by silica gel chromatography (NH—Si, 50% ethyl acetate/hexane) to give the object product (64.5 mg, 52%).

¹H NMR (300 MHz, DMSO-d₆) δ ppm 5.43 (s, 4H) 7.04 (d, J=7.95 Hz, 1H) 7.27-7.43 (m, 4H) 7.57 (t, J=7.76 Hz, 1H) 7.71 (d, J=5.30 Hz, 1H) 7.73-7.81 (m, 1H) 7.82 (s, 1H) 8.19 (s, 1H) 8.27 (s, 1H) 8.54 (d, J=4.16 Hz, 1H) 8.73 (d, J=4.92 Hz, 1H) 11.01 (s, 1H)

### Example 204

### N-[1-(4-fluorobenzyl)-1H-pyrazol-4-yl]-4-{[2-(trifluoromethyl)phenoxy]methyl}pyridine-2-carboxamide

### (1) ethyl 4-{[2-(trifluoromethyl)phenoxy]methyl}pyridine-2-carboxylate

A solution of ethyl 4-(hydroxymethyl)pyridine-2-carboxylate (5 g), o-trifluoromethylphenol (4.92 g), triphenylphosphine (8.68 g) and diethyl azodicarboxylate (40% toluene solution, 15 mL) in tetrahydrofuran (200 mL) was stirred at 70° C. overnight. After cooling to room temperature, the solvent was evaporated under reduced pressure, and the residue was dissolved in ethyl acetate. The organic layer was washed with saturated aqueous sodium hydrogen carbonate solution and saturated brine and dried over magnesium sulfate. The desiccant was filtered off and the filtrate was concentrated. The residue was purified by silica gel chromatography (Si, 30% to 50% ethyl acetate/hexane) to give the object product (6.83 g).

¹H NMR (300 MHz, DMSO-d₆) δ ppm 1.34 (t, J=7.06 Hz, 3H) 4.36 (q, J=7.16 Hz, 2H) 5.47 (s, 2H) 7.16 (t, J=7.54 Hz, 1H) 7.32 (d, J=8.29 Hz, 1H) 7.60-7.72 (m, 3H) 8.17 (d, J=0.75 Hz, 1H) 8.75 (d, J=4.90 Hz, 1H)

### (2) 4-{[2-(trifluoromethyl)phenoxy]methyl}pyridine-2-carboxylic acid

Ethyl 4-{[2-(trifluoromethyl)phenoxy]methyl}pyridine-2-carboxylate (6.83 g) obtained in the above-mentioned reaction was dissolved in a mixed solution of methanol (50 mL) and tetrahydrofuran (50 mL), 3N aqueous sodium hydroxide (100 mL) solution was added. The mixed solution was stirred at room temperature for 3 hr. The solvent was evaporated under reduced pressure, and the residue was dissolved in ethyl acetate. The organic layer was washed with 10% aqueous citric acid solution and dried over magnesium sulfate. The desiccant was filtered off and the filtrate was concentrated. The precipitated crystal was washed with hexane to give the object product (3.73 g, 60%). The object product was used for the next reaction without further purification.

### (3) tert-butyl 4-{[(4-{[2-(trifluoromethyl)phenoxy]methyl}pyridin-2-yl)carbonyl]amino}-1H-pyrazole-1-carboxylate

Using 4-{[2-(trifluoromethyl)phenoxy]methyl}pyridine-2-carboxylic acid (1.0 g) obtained in the above-mentioned reaction and in the same manner as in Example 203(1), the object product (1.11 g, 71%) was obtained.

¹H NMR (300 MHz, DMSO-d₆) δ ppm 1.60 (s, 9H) 5.51 (s, 2H) 7.16 (t, J=7.57 Hz, 1H) 7.34 (d, J=8.33 Hz, 1H) 7.62-7.73 (m, 3H) 8.15 (s, 1H) 8.23 (s, 1H) 8.57 (s, 1H) 8.78 (d, J=4.92 Hz, 1H) 11.28 (s, 1H)

### (4) N-1H-pyrazol-4-yl-4-{[2-(trifluoromethyl)phenoxy]methyl}pyridine-2-carboxamide dihydrochloride

In the same manner as in Example 203(2) and using tert-butyl 4-{[(4-{[2-(trifluoromethyl)phenoxy]methyl}pyridin-2-yl)carbonyl]amino}-1H-pyrazole-1-carboxylate (1.11 g) obtained in the above-mentioned reaction, the object product (0.81 g) was obtained.

¹H NMR (300 MHz, DMSO-d₆) δ ppm 5.50 (s, 2H) 7.16 (t, J=7.54 Hz, 1H) 7.33 (d, J=8.29 Hz, 1H) 7.59-7.74 (m, 3H) 7.85 (br. s., 1H) 8.08 (br. s., 1H) 8.21 (s, 1H) 8.75 (d, J=4.90 Hz, 1H) 10.91 (s, 1H) 12.64 (br. s., 1H)

### (5) N-[1-(4-fluorobenzyl)-1H-pyrazol-4-yl]-4-{[2-(trifluoromethyl)phenoxy]methyl}pyridine-2-carboxamide

In the same manner as in Example 203(3) and using N-1H-pyrazol-4-yl-4-{[2-(trifluoromethyl)phenoxy]methyl}pyridine-2-carboxamide dihydrochloride (100 mg) obtained in the above-mentioned reaction and p-fluorobenzylbromide (62 mg), the object product (68.2 mg, 52%) was obtained.

¹H NMR (300 MHz, DMSO-d₆) δ ppm 5.31 (s, 2H) 5.49 (s, 2H) 7.09-7.23 (m, 3H) 7.25-7.37 (m, 3H) 7.59-7.73 (m, 3H) 7.78 (s, 1H) 8.20 (s, 1H) 8.26 (s, 1H) 8.74 (d, J=5.09 Hz, 1H) 10.99 (s, 1H)

### Example 205

### N-[1-(pyridin-2-ylmethyl)-1H-pyrazol-4-yl]-4-{[2-(trifluoromethyl)phenoxy]methyl}pyridine-2-carboxamide

Using N-1H-pyrazol-4-yl-4-{[2-(trifluoromethyl)phenoxy]methyl}pyridine-2-carboxamide dihydrochloride (100 mg) obtained in Example 204(4) and 2-bromomethylpyridine and in the same manner as in Example 203(3), the object product (47.5 mg, 38%) was obtained.

¹H NMR (300 MHz, DMSO-d₆) δ ppm 5.42 (s, 2H) 5.50 (s, 2H) 7.04 (d, J=7.57 Hz, 1H) 7.15 (t, J=7.38 Hz, 1H) 7.26-7.42 (m, 2H) 7.58-7.91 (m, 5H) 8.21 (s, 1H) 8.29 (s, 1H) 8.54 (d, J=4.16 Hz, 1H) 8.75 (d, J=4.92 Hz, 1H) 11.01 (s, 1H)

### Example 206

### 2-[(2-chloro-5-fluorophenoxy)methyl]-N-[4-(1H-pyrazol-1-ylmethyl)phenyl]morpholine-4-carboxamide

### (1) phenyl [4-(1H-pyrazol-1-ylmethyl)phenyl]carbamate

By reaction in the same manner as in Example 202(1) and using 4-(1H-pyrazol-1-ylmethyl)aniline (0.5 g), the object product (0.6 g) was obtained as a white solid.

¹H NMR (300 MHz, CDCl₃) δ ppm 5.28 (s, 2H) 6.27 (m, 1H) 7.06 (brs, 1H) 7.15-7.26 (m, 5H) 7.35-7.42 (m, 5H) 7.54 (m, 1H)

### (2) 2-[(2-chloro-5-fluorophenoxy)methyl]-N-[4-(1H-pyrazol-1-ylmethyl)phenyl]morpholine-4-carboxamide

By reaction in the same manner as in Example 202(2) and using phenyl [4-(1H-pyrazol-1-ylmethyl)phenyl]carbamate (0.2 g) obtained in the above-mentioned reaction and 2-[(2-chloro-5-fluorophenoxy)methyl]morpholine hydrochloride (0.2 g) synthesized by a method similar to that in Example 18, the object product (0.14 g) was obtained as a pale-yellow oil.

¹H NMR (300 MHz, CDCl₃) δ ppm 3.04-3.20 (m, 2H) 3.66 (m, 1H) 3.84-3.92 (m, 2H) 3.98-4.15 (m, 4H) 5.25 (s, 2H) 6.26 (m, 1H) 6.56-6.70 (m, 3H) 7.13 (d, J=8.4 Hz, 2H) 7.25-7.35 (m, 4H) 7.52 (m, 1H)

### Example 207

### N-[1-(2-hydroxybutyl)-1H-pyrazol-4-yl]-4-{[2-(trifluoromethyl)phenoxy]methyl}pyridine-2-carboxamide

To a solution of N-1H-pyrazol-4-yl-4-{[2-(trifluoromethyl)phenoxy]methyl}pyridine-2-carboxamide dihydrochloride (100 mg) obtained in Example 204(4) in dimethylformamide (2 mL) was added sodium hydride (22 mg) under ice-cooling, and the mixture was stirred for 30 min. 1-Bromo-2-butanol (50 mg) was added to the reaction solution, and the mixture was stirred at 80° C. overnight. After cooling to room temperature, the reaction solution was poured into water, and the mixture was extracted with ethyl acetate. The organic layer was washed with saturated brine, and dried over sodium sulfate. The desiccant was filtered off and the filtrate was concentrated. The residue was purified by silica gel chromatography (NH—Si, ethyl acetate) and further recrystallized to give the object product (22.4 mg, 18.7%).

¹H NMR (300 MHz, DMSO-d₆) δ ppm 0.89 (t, J=7.44 Hz, 3H) 1.19-1.45 (m, 2H) 3.62-3.76 (m, 1H) 3.89-4.11 (m, 2H) 4.85 (d, J=5.46 Hz, 1H) 5.49 (s, 2H) 7.16 (t, J=7.54 Hz, 1H) 7.33 (d, J=8.29 Hz, 1H) 7.58-7.72 (m, 3H) 7.75 (s, 1H) 8.13 (s, 1H) 8.21 (s, 1H) 8.74 (d, J=5.09 Hz, 1H) 10.93 (s, 1H)

### Example 208

### N-[1-(2-hydroxybutyl)-1H-pyrazol-4-yl]-4-{[3-(trifluoromethyl)phenoxy]methyl}pyridine-2-carboxamide

Using N-1H-pyrazol-4-yl-4-{[3-(trifluoromethyl)phenoxy]methyl}pyridine-2-carboxamide dihydrochloride (100 mg) obtained in Example 203(2) and in the same manner as in Example 207, the object product (36.5 mg, 28%) was obtained.

¹H NMR (300 MHz, DMSO-d₆) δ ppm 0.89 (t, J=7.38 Hz, 3H) 1.14-1.47 (m, 2H) 3.60-3.77 (m, 1H) 3.90-4.12 (m, 2H) 4.86 (d, J=5.68 Hz, 1H) 5.43 (s, 2H) 7.25-7.47 (m, 3H) 7.57 (t, J=7.76 Hz, 1H) 7.71 (d, J=4.54 Hz, 1H) 7.76 (s, 1H) 8.12 (s, 1H) 8.19 (s, 1H) 8.72 (d, J=4.92 Hz, 1H) 10.94 (s, 1H)

### Example 209

### 2-[(2-chloro-5-fluorophenoxy)methyl]-N-[5-(4-fluorophenoxy)pyridin-2-yl]morpholine-4-carboxamide

### (1) phenyl [5-(4-fluorophenoxy)pyridin-2-yl]carbamate

By reaction in the same manner as in Example 202(1) and using 5-(4-fluorophenoxy)pyridin-2-amine (0.28 g), the object product (0.4 g) was obtained as a white solid.

¹H NMR (300 MHz, CDCl₃) δ ppm 6.93-6.99 (m, 4H) 7.11-7.15 (m, 2H) 7.28 (m, 1H) 7.36-7.42 (m, 3H) 8.01 (d, J=9.0 Hz, 1H) 8.11 (m, 1H) 9.25 (brs, 1H)

### (2) 2-[(2-chloro-5-fluorophenoxy)methyl]-N-[5-(4-fluorophenoxy)pyridin-2-yl]morpholine-4-carboxamide

By reaction in the same manner as in Example 202(2) and using phenyl [5-(4-fluorophenoxy)pyridin-2-yl]carbamate (0.23 g) obtained in the above-mentioned reaction and 2-[(2-chloro-5-fluorophenoxy)methyl]morpholine hydrochloride (0.2 g) synthesized by a method similar to that in Example 18, the object product (0.13 g) was obtained as a pale-yellow oil.

¹H NMR (300 MHz, CDCl₃) δ ppm 3.06-3.22 (m, 2H) 3.68 (m, 1H) 3.88-3.92 (m, 2H) 4.01-4.19 (m, 4H) 6.61-6.71 (m, 2H) 6.91-7.04 (m, 4H) 7.26-7.32 (m, 2H) 7.43 (m, 1H) 7.96-7.99 (m, 2H)

### Example 210

### 2-[(2-chloro-5-fluorophenoxy)methyl]-N-(5-methoxypyridin-2-yl)morpholine-4-carboxamide

(1) phenyl (5-methoxypyridin-2-yl)carbamate

By reaction in the same manner as in Example 202(1) and using 5-methoxypyridin-2-amine (0.8 g), the object product (0.2 g) was obtained as a white solid.

¹H NMR (300 MHz, CDCl₃) δ ppm 3.81 (s, 2H) 7.19-7.31 (m, 4H) 7.37-7.43 (m, 2H) 7.95 (d, J=9.0 Hz, 1H) 8.05 (m, 1H) 8.86 (brs, 1H)

### (2) 2-[(2-chloro-5-fluorophenoxy)methyl]-N-(5-methoxypyridin-2-yl)morpholine-4-carboxamide

By reaction in the same manner as in Example 202(2) and using phenyl (5-methoxypyridin-2-yl)carbamate (0.10 g) obtained in the above-mentioned reaction and 2-[(2-chloro-5-fluorophenoxy)methyl]morpholine hydrochloride (0.12 g) synthesized by a method similar to that in Example 18, the object product (0.07 g) was obtained as a pale-yellow oil.

¹H NMR (300 MHz, CDCl₃) δ ppm 3.05-3.22 (m, 2H) 3.69 (m, 1H) 3.82 (s, 3H) 3.86-3.95 (m, 2H) 4.00-4.18 (m, 4H) 6.62-6.71 (m, 2H) 7.22-7.32 (m, 3H) 7.89-7.94 (m, 2H)

### Example 211

### 2-[(2-chloro-5-fluorophenoxy)methyl]-N-[6-(4-fluorophenoxy)pyridin-3-yl]morpholine-4-carboxamide

### (1) phenyl [6-(4-fluorophenoxy)pyridin-3-yl]carbamate

By reaction in the same manner as in Example 202(1) and using 6-(4-fluorophenoxy)pyridin-3-amine (0.70 g), the object product (0.68 g) was obtained as a white solid.

¹H NMR (300 MHz, CDCl₃) δ ppm 6.90 (d, J=9.0 Hz, 1H) 7.02 (brs, 1H) 7.06-7.16 (m, 4H) 7.18-7.25 (m, 3H) 7.37-7.43 (m, 2H) 8.01 (m, 1H) 8.12 (d, J=2.7 Hz, 1H)

### (2) 2-[(2-chloro-5-fluorophenoxy)methyl]-N-[6-(4-fluorophenoxy)pyridin-3-yl]morpholine-4-carboxamide

By reaction in the same manner as in Example 202(2) and using phenyl [6-(4-fluorophenoxy)pyridin-3-yl]carbamate (0.25 g) obtained in the above-mentioned reaction and 2-[(2-chloro-5-fluorophenoxy)methyl]morpholine hydrochloride (0.22 g) synthesized by a method similar to that in Example 18, the object product (0.10 g) was obtained as a pale-yellow oil.

¹H NMR (300 MHz, CDCl₃) δ ppm 3.01-3.16 (m, 2H) 3.63 (m, 1H) 3.83-3.88 (m, 2H) 3.94-3.98 (m, 2H) 3.99-4.12 (m, 2H) 6.61-6.69 (m, 2H) 6.81 (d, J=5.7 Hz, 1H) 6.93 (s, 1H) 7.02-7.04 (m, 4H) 7.28 (m, 1H) 7.86 (m, 1H) 7.95 (d, J=2.1 Hz, 1H)

### Example 212

### 2-[(2-chloro-5-fluorophenoxy)methyl]-N-[5-(hydroxymethyl)pyridin-2-yl]morpholine-4-carboxamide

### (1) phenyl [5-({[tert-butyl(dimethyl)silyl]oxy}methyl)pyridin-2-yl]carbamate

By reaction in the same manner as in Example 202(1) and using 5-({[tert-butyl(dimethyl)silyl]oxy}methyl)pyridin-2-amine (1.7 g), the object product (1.4 g) was obtained as a white solid.

¹H NMR (300 MHz, CDCl₃) δ ppm 0.06 (s, 6H) 0.89 (s, 9H) 4.68 (s, 2H) 7.20-7.28 (m, 3H) 7.38-7.44 (m, 2H) 7.68 (dd, J=2.4, 8.7 Hz, 1H) 8.01 (d, J=8.1 Hz, 1H) 8.33 (m, 1H) 9.54 (brs, 1H)

### (2) N-[5-({[tert-butyl(dimethyl)silyl]oxy}methyl)pyridin-2-yl]-2-[(2-chloro-5-fluorophenoxy)methyl]morpholine-4-carboxamide

By reaction in the same manner as in Example 202(2) and using phenyl [5-({[tert-butyl(dimethyl)silyl]oxy}methyl)pyridin-2-yl]carbamate (0.5 g) obtained in the above-mentioned reaction and 2-[(2-chloro-5-fluorophenoxy)methyl]morpholine hydrochloride (0.39 g) synthesized by a method similar to that in Example 18, the object product (0.52 g) was obtained as a pale-yellow oil.

¹H NMR (300 MHz, CDCl₃) δ ppm 0.09 (s, 6H) 0.93 (s, 9H) 3.06-3.23 (m, 2H) 3.69 (m, 1H) 3.88 (m, 2H) 4.01-4.19 (m, 4 H) 4.68 (s, 2H) 6.62-6.71 (m, 2H) 7.26-7.32 (m, 2H) 7.61 (dd, J=2.1, 8.4 Hz, 1H) 7.97 (d, J=8.7 Hz, 1H) 8.16 (brs, 1H)

### (3) 2-[(2-chloro-5-fluorophenoxy)methyl]-N-[5-(hydroxymethyl)pyridin-2-yl]morpholine-4-carboxamide

To a solution of N-[5-({[tert-butyl(dimethyl)silyl]oxy}methyl)pyridin-2-yl]-2-[(2-chloro-5-fluorophenoxy)methyl]morpholine-4-carboxamide (0.44 g) obtained in the above-mentioned reaction in tetrahydrofuran (10 mL) was added tetrabutylammonium fluoride (1.0M tetrahydrofuran solution, 1 mL), and the mixture was stirred at room temperature for 30 min. The solvent was evaporated under reduced pressure, and the residue was purified by silica gel column (ethyl acetate to 5% ethyl acetate/methanol) to give the object product (0.23 g).

¹H NMR (300 MHz, CDCl₃) δ ppm 3.03-3.18 (m, 2H) 3.48 (brs, 1H) 3.65 (m, 1H) 3.86-3.92 (m, 2H) 3.97-4.15 (m, 4H) 4.60 (s, 2H) 6.61-6.70 (m, 2H) 7.28 (m, 1H) 7.62 (dd, J=2.4, 8.7 Hz, 1H) 7.68 (brs, 1H) 7.92 (d, J=8.7 Hz, 1H) 8.08 (d, J=1.8 Hz, 1H)

### Example 213

### 4-[(2,5-dichlorophenoxy)methyl]-N-[1-(2-hydroxybutyl)-1H-pyrazol-4-yl]pyridine-2-carboxamide

### (1) 1-(4-nitro-1H-pyrazol-1-yl)butan-2-ol

Using 4-nitro-1H-pyrazole (500 mg) and in the same manner as in Example 207, the object product (0.68 g) was obtained.

¹H NMR (300 MHz, DMSO-d₆) δ ppm 0.90 (t, J=7.44 Hz, 3H) 1.22-1.52 (m, 2H) 3.75 (ddd, J=2.73, 4.57, 12.29 Hz, 1H) 3.95-4.24 (m, 2H) 4.99 (d, J=5.65 Hz, 1H) 8.26 (s, 1H) 8.76 (s, 1H)

### (2) 1-(4-amino-1H-pyrazol-1-yl)butan-2-ol

1-(4-Nitro-1H-pyrazol-1-yl)butan-2-ol (0.62 g) obtained in the above-mentioned reaction was dissolved in methanol (30 mL) solution, 5% palladium carbon was added, and the mixture was stirred overnight under a hydrogen atmosphere at room temperature. The catalyst was filtered off through celite and the filtrate was concentrated. The residue was purified by silica gel chromatography (NH—Si, ethyl acetate to 5% methanol/ethyl acetate) to give the object product (463 mg, 89%).

¹H NMR (300 MHz, CDCl₃) δ ppm 0.99 (t, J=7.38 Hz, 3H) 1.40-1.53 (m, 2H) 3.87 (d, J=9.09 Hz, 2H) 4.00-4.09 (m, 1H) 7.04 (s, 1H) 7.16 (s, 1H)

### (3) 4-[(2,5-dichlorophenoxy)methyl]-N-[1-(2-hydroxybutyl)-1H-pyrazol-4-yl]pyridine-2-carboxamide

In the same manner as in Example 203(1) and using 4-[(2,5-dichlorophenoxy)methyl]pyridine-2-carboxylic acid (92 mg) obtained in Example 112(2) and 1-(4-amino-1H-pyrazol-1-yl)butan-2-ol (60 mg) obtained in the above-mentioned reaction, the object product (69 mg, 51%) was obtained.

¹H NMR (300 MHz, DMSO-d₆) δ ppm 0.89 (t, J=7.35 Hz, 3H) 1.14-1.46 (m, 2H) 3.60-3.76 (m, 1H) 3.91-4.12 (m, 2H) 4.85 (d, J=5.27 Hz, 1H) 5.48 (s, 2H) 7.10 (dd, J=2.26, 8.48 Hz, 1H) 7.38 (d, J=2.07 Hz, 1H) 7.54 (d, J=8.48 Hz, 1H) 7.68 (d, J=3.58 Hz, 1H) 7.76 (s, 1H) 8.13 (s, 1H) 8.21 (s, 1H) 8.75 (d, J=4.90 Hz, 1H) 10.95 (s, 1H)

### Example 214

### N-[1-(4-fluorobenzyl)-1H-pyrazol-4-yl]-4-{[2-fluoro-5-(trifluoromethyl)phenoxy]methyl}pyridine-2-carboxamide

### (1) ethyl 4-{[2-fluoro-5-(trifluoromethyl)phenoxy]methyl}pyridine-2-carboxylate

Using ethyl 4-(hydroxymethyl)pyridine-2-carboxylate (1.0 g) and 2-fluoro-5-(trifluoromethyl)phenol (1.1 g) and in the same manner as in Example 204(1), the object product (1.04 g, 55%) was obtained.

¹H NMR (300 MHz, DMSO-d₆) δ ppm 1.34 (t, J=7.19 Hz, 3H) 4.37 (q, J=6.94 Hz, 2H) 5.48 (s, 2H) 7.36-7.45 (m, 1H) 7.48-7.57 (m, 1H) 7.60-7.67 (m, 1H) 7.68-7.74 (m, 1H) 8.15 (s, 1H) 8.76 (d, J=4.92 Hz, 1H)

### (2) 4-{[2-fluoro-5-(trifluoromethyl)phenoxy]methyl}pyridine-2-carboxylic acid

Using ethyl 4-{[2-fluoro-5-(trifluoromethyl)phenoxy]methyl}pyridine-2-carboxylate (1.04 g) obtained in the above-mentioned reaction and in the same manner as in Example 204(2), the object product (880 mg, 92%) was obtained.

¹H NMR (300 MHz, DMSO-d₆) δ ppm 5.48 (s, 2H) 7.34-7.47 (m, 1H) 7.48-7.58 (m, 1H) 7.60-7.72 (m, 2H) 8.13 (s, 1H) 8.74 (d, J=4.92 Hz, 1H) 13.28 (brs, 1H)

### (3) N-[1-(4-fluorobenzyl)-1H-pyrazol-4-yl]-4-{[2-fluoro-5-(trifluoromethyl)phenoxy]methyl}pyridine-2-carboxamide

In the same manner as in Example 203(1) and using 4-{[2-fluoro-5-(trifluoromethyl)phenoxy]methyl}pyridine-2-carboxylic acid (82 mg) obtained in the above-mentioned reaction and 1-(4-fluorobenzyl)-1H-pyrazol-4-amine (50 mg), the object product (108 mg, 85%) was obtained.

¹H NMR (300 MHz, DMSO-d₆) δ ppm 5.30 (s, 2H) 5.50 (s, 2H) 7.09-7.23 (m, 2H) 7.25-7.35 (m, 2H) 7.36-7.45 (m, 1H) 7.47-7.58 (m, 1H) 7.59-7.72 (m, 2H) 7.78 (s, 1H) 8.18 (s, 1H) 8.23 (s, 1H) 8.73 (d, J=4.90 Hz, 1H) 10.99 (s, 1H)

### Example 215

### 4-{[2-chloro-5-(trifluoromethyl)phenoxy]methyl}-N-[1-(4-fluorobenzyl)-1H-pyrazol-4-yl]pyridine-2-carboxamide

### (1) ethyl 4-{[2-chloro-5-(trifluoromethyl)phenoxy]methyl}pyridine-2-carboxylate

Using ethyl 4-(hydroxymethyl)pyridine-2-carboxylate (1.0 g) and 2-chloro-5-(trifluoromethyl)phenol (1.2 g) and in the same manner as in Example 204(1), the object product (1.42 g, 72%) was obtained.

¹H NMR (300 MHz, DMSO-d₆) δ ppm 1.34 (t, J=7.00 Hz, 3H) 4.37 (q, J=6.94 Hz, 2H) 5.52 (s, 2H) 7.40 (d, J=8.33 Hz, 1H) 7.58 (d, J=1.89 Hz, 1H) 7.68-7.80 (m, 2H) 8.18 (s, 1H) 8.76 (d, J=4.92 Hz, 1H)

### (2) 4-{[2-chloro-5-(trifluoromethyl)phenoxy]methyl}pyridine-2-carboxylic acid

In the same manner as in Example 204(2) and using ethyl 4-{[2-chloro-5-(trifluoromethyl)phenoxy]methyl}pyridine-2-carboxylate (1.42 g) obtained in the above-mentioned reaction, the object product (1.26 g, 96%) was obtained.

¹H NMR (300 MHz, DMSO-d₆) δ ppm 5.51 (s, 2H) 7.35-7.45 (m, 1H) 7.58 (d, J=1.51 Hz, 1H) 7.70 (dd, J=1.89, 4.92 Hz, 1H) 7.72-7.79 (m, 1H) 8.16 (d, J=0.76 Hz, 1H) 8.75 (dd, J=0.76, 4.92 Hz, 1H) 13.26 (brs, 1H)

### (3) 4-{[2-chloro-5-(trifluoromethyl)phenoxy]methyl}-N-[1-(4-fluorobenzyl)-1H-pyrazol-4-yl]pyridine-2-carboxamide

In the same manner as in Example 203(1) and using 4-{[2-chloro-5-(trifluoromethyl)phenoxy]methyl}pyridine-2-carboxylic acid (86 mg) obtained in the above-mentioned reaction and 1-(4-fluorobenzyl)-1H-pyrazol-4-amine (50 mg), the object product (107 mg, 84%) was obtained.

¹H NMR (300 MHz, DMSO-d₆) δ ppm 5.31 (s, 2H) 5.55 (s, 2H) 7.09-7.24 (m, 2H) 7.31 (t, J=5.75 Hz, 2H) 7.39 (d, J=7.72 Hz, 1H) 7.58 (brs, 1H) 7.67-7.83 (m, 3H) 8.24 (d, J=5.46 Hz, 2H) 8.75 (d, J=4.90 Hz, 1H) 10.99 (s, 1H)

### Example 216

### 2-[(2-chloro-5-fluorophenoxy)methyl]-N-(6-methylpyridin-2-yl) morpholine-4-carboxamide

### (1) phenyl (6-methylpyridin-2-yl)carbamate

By reaction in the same manner as in Example 202(1) and using 6-methylpyridin-2-amine (3.0 g), the object product (5.0 g) was obtained as a yellow oil.

¹H NMR (300 MHz, CDCl₃) δ ppm 2.51 (s, 3H) 6.79-6.90 (m, 3H) 7.13-7.24 (m, 2H) 7.34-7.39 (m, 2H) 7.58 (m, 1H) 7.81 (brd, J=8.1 Hz, 1H)

### (2) 2-[(2-chloro-5-fluorophenoxy)methyl]-N-(6-methylpyridin-2-yl)morpholine-4-carboxamide

By reaction in the same manner as in Example 202(2) and using phenyl (6-methylpyridin-2-yl)carbamate (0.24 g) obtained in the above-mentioned reaction and 2-[(2-chloro-5-fluorophenoxy)methyl]morpholine hydrochloride (0.3 g) synthesized by a method similar to that in Example 18, the object product (0.19 g) was obtained as a pale-yellow oil.

¹H NMR (300 MHz, CDCl₃) δ ppm 2.41 (s, 3H) 3.05-3.21 (m, 2H) 3.63 (m, 1H) 3.87-3.93 (m, 2H) 4.00-4.19 (m, 4H) 6.61-6.81 (m, 3H) 7.27-7.34 (m, 2H) 7.52 (m, 1H) 7.79 (brd, J=7.2 Hz, 1H)

### Example 217

### 2-[(2-chloro-5-fluorophenoxy)methyl]-N-(3-methyl-1,2,4-thiadiazol-5-yl)morpholine-4-carboxamide

### (1) phenyl (3-methyl-1,2,4-thiadiazol-5-yl)carbamate

By reaction in the same manner as in Example 202(1) and using 3-methyl-1,2,4-thiadiazole-5-amine (2.0 g), the object product (1.1 g) was obtained as a yellow oil.

¹H NMR (300 MHz, CDCl₃) δ ppm 2.62 (s, 3H) 7.20-7.48 (m, 5H) 12.4 (brs, 1H)

### (2) 2-[(2-chloro-5-fluorophenoxy)methyl]-N-(3-methyl-1,2,4-thiadiazol-5-yl)morpholine-4-carboxamide

By reaction in the same manner as in Example 202(2) and using phenyl (3-methyl-1,2,4-thiadiazol-5-yl)carbamate (0.3 g) obtained in the above-mentioned reaction and 2-[(2-chloro-5-fluorophenoxy)methyl]morpholine hydrochloride (0.36 g) synthesized by a method similar to that in Example 18, the object product (0.22 g) was obtained as a white solid.

¹H NMR (300 MHz, CDCl₃) δ ppm 2.50 (s, 3H) 3.15-3.25 (m, 2H) 3.66 (m, 1H) 3.89-3.96 (m, 2H) 4.01-4.17 (m, 4H) 6.63-6.70 (m, 2H) 7.31 (m, 1H) 9.33 (brs, 1H)

### Example 218

### 2-[(2-chloro-5-fluorophenoxy)methyl]-N-(4-methyl-1,3-thiazol-2-yl)morpholine-4-carboxamide

### (1) phenyl (4-methyl-1,3-thiazol-2-yl)carbamate

By reaction in the same manner as in Example 202(1) and using 4-methyl-1,3-thiazol-2-amine (2.0 g), the object product (1.4 g) was obtained as a yellow oil.

¹H NMR (300 MHz, CDCl₃) δ ppm 2.41 (s, 3H) 6.52 (s, 1H) 7.18-7.29 (m, 3H) 7.38-7.44 (m, 2-H) 12.0 (brs, 1H)

### (2) 2-[(2-chloro-5-fluorophenoxy)methyl]-N-(4-methyl-1,3-thiazol-2-yl)morpholine-4-carboxamide

By reaction in the same manner as in Example 202(2) and using phenyl (4-methyl-1,3-thiazol-2-yl)carbamate (0.25 g) obtained in the above-mentioned reaction and 2-[(2-chloro-5-fluorophenoxy)methyl]morpholine hydrochloride (0.3 g) synthesized by a method similar to that in Example 18, the object product (0.23 g) was obtained as a white solid.

¹H NMR (300 MHz, CDCl₃) δ ppm 2.28 (s, 3H) 3.04-3.19 (m, 2H) 3.68 (m, 1H) 3.85-4.21 (m, 6H) 6.34 (brs, 1H) 6.61-6.70 (m, 3H) 7.30 (m, 1H)

### Example 219

### 2-[(2-chloro-5-fluorophenoxy)methyl]-N-(5,6-dimethylpyridin-2-yl)morpholine-4-carboxamide

### (1) phenyl (5,6-dimethylpyridin-2-yl)carbamate

By reaction in the same manner as in Example 202(1) and using 5,6-dimethylpyridin-2-amine (2.0 g), the object product (3.1 g) was obtained as a white solid.

¹H NMR (300 MHz, CDCl₃) δ ppm 2.23 (s, 3H) 2.42 (s, 3H) 7.15-7.25 (m, 3H) 7.36-7.43 (m, 3H) 7.68 (d, J=8.1 Hz, 1H) 8.03 (brs, 1H)

### (2) 2-[(2-chloro-5-fluorophenoxy)methyl]-N-(5,6-dimethylpyridin-2-yl)morpholine-4-carboxamide

By reaction in the same manner as in Example 202(2) and using phenyl (5,6-dimethylpyridin-2-yl)carbamate (0.3 g) obtained in the above-mentioned reaction and 2-[(2-chloro-5-fluorophenoxy)methyl]morpholine hydrochloride (0.35 g) synthesized by a method similar to that in Example 18, the object product (0.25 g) was obtained as a white solid.

¹H NMR (300 MHz, CDCl₃) δ ppm 2.21 (s, 3H) 2.36 (s, 3H) 3.08-3.17 (m, 2H) 3.68 (m, 1H) 3.88-3.92 (m, 2H) 4.01-4.18 (m, 4H) 6.61-6.71 (m, 2H) 7.13 (brs, 1H) 7.27-7.39 (m, 2H) 7.71 (brd, J=8.7 Hz, 1H)

### Example 220

### 4-[(2,5-dichlorophenoxy)methyl]-N-(3-methyl-1,2,4-thiadiazol-5-yl)pyridine-2-carboxamide

In the same manner as in Example 203(1) and using 4-[(2,5-dichlorophenoxy)methyl]pyridine-2-carboxylic acid (129 mg) and 3-methyl-1,2,4-thiadiazol-5-amine (50 mg), the object product (49 mg, 29%) was obtained.

¹H NMR (300 MHz, DMSO-d₆) δ ppm 2.51 (s, 3H) 5.50 (s, 2H) 7.04-7.16 (m, 1H) 7.39 (d, J=2.27 Hz, 1H) 7.54 (d, J=8.33 Hz, 1H) 7.78 (d, J=5.30 Hz, 1H) 8.28 (s, 1H) 8.83 (d, J=4.92 Hz, 1H) 13.26 (brs, 1H)

### Example 221

### 2-[(2-chloro-5-fluorophenoxy)methyl]-N-[6-(hydroxymethyl)pyridin-2-yl]morpholine-4-carboxamide

### (1) phenyl [6-({[tert-butyl(dimethyl)silyl]oxy}methyl)pyridin-2-yl]carbamate

By reaction in the same manner as in Example 202(1) and using 6-({[tert-butyl(dimethyl)silyl]oxy}methyl)pyridin-2-amine (1.2 g), the object product (1.6 g) was obtained as a white solid.

¹H NMR (300 MHz, CDCl₃) δ ppm 0.13 (s, 6H) 0.96 (s, 9H) 4.72 (s, 2H) 7.17-7.25 (m, 4H) 7.37-7.42 (m, 2H) 7.66 (brs, 1H) 7.72 (t, J=7.5 Hz, 1H) 7.82 (d, J=8.4 Hz, 1H)

### (2) N-[6-({[tert-butyl(dimethyl)silyl]oxy}methyl)pyridin-2-yl]-2-[(2-chloro-5-fluorophenoxy)methyl]morpholine-4-carboxamide

By reaction in the same manner as in Example 202(2) and using phenyl [6-({[tert-butyl(dimethyl)silyl]oxy}methyl)pyridin-2-yl]carbamate (0.41 g) obtained in the above-mentioned reaction and 2-[(2-chloro-5-fluorophenoxy)methyl]morpholine hydrochloride (0.32 g) synthesized by a method similar to that in Example 18, the object product (0.50 g) was obtained as a white solid.

¹H NMR (300 MHz, CDCl₃) δ ppm 0.10 (s, 6H) 0.96 (s, 9H) 3.07-3.15 (m, 2H) 3.69 (m, 1H) 3.88 (m, 2H) 4.02-4.11 (m, 4H) 4.67 (s, 2H) 6.62 (m, 2H) 7.10-7.16 (m, 2H) 7.30 (dd, J=5.7, 8.7 Hz, 1H) 7.67 (m, 1H) 7.85 (d, J=7.8 Hz, 1H)

### (3) 2-[(2-chloro-5-fluorophenoxy)methyl]-N-[6-(hydroxymethyl)pyridin-2-yl]morpholine-4-carboxamide

By reaction in the same manner as in Example 212(3) and using N-[6-({[tert-butyl(dimethyl)silyl]oxy}methyl)pyridin-2-yl]-2-[(2-chloro-5-fluorophenoxy)methyl]morpholine-4-carboxamide (0.50 g) obtained in the above-mentioned reaction, the object product (0.20 g) was obtained as a white solid.

¹H NMR (300 MHz, CDCl₃) δ ppm 2.81-2.99 (m, 2H) 3.51 (m, 1H) 3.77 (m, 1H) 3.93 (m, 1H) 4.15-4.20 (m, 4H) 4.46 (d, J=5.1 Hz, 2H) 5.33 (d, J=6.3 Hz, 1H) 6.81 (m, 1H) 7.06 (d, J=6.9 Hz, 1H) 7.16 (dd, J=5.7, 10.5 Hz, 1H) 7.46 (dd, J=6.3, 8.7 Hz, 1H) 7.62-7.70 (m, 2H) 9.16 (s, 1H)

### Example 222

### 2-[(2-chloro-5-fluorophenoxy)methyl]-N-(3-ethyl-1,2,4-thiadiazol-5-yl)morpholine-4-carboxamide

### (1) phenyl (3-ethyl-1,2,4-thiadiazol-5-yl)carbamate

By reaction in the same manner as in Example 202(1) and using 3-ethyl-1,2,4-thiadiazol-5-amine (1.0 g), the object product (0.8 g) was obtained as a white solid.

¹H NMR (300 MHz, CDCl₃) δ ppm 1.34 (t, J=7.8 Hz, 3H) 2.98 (q, J=7.8 Hz, 2H) 7.21-7.24 (m, 2H) 7.34 (m, 1H) 7.45-7.48 (m, 2H) 12.0 (brs, 1H)

### (2) 2-[(2-chloro-5-fluorophenoxy)methyl]-N-(3-ethyl-1,2,4-thiadiazol-5-yl)morpholine-4-carboxamide

By reaction in the same manner as in Example 202(2) and using phenyl (3-ethyl-1,2,4-thiadiazol-5-yl)carbamate (0.27 g) obtained in the above-mentioned reaction and 2-[(2-chloro-5-fluorophenoxy)methyl]morpholine hydrochloride (0.30 g) synthesized by a method similar to that in Example 18, the object product (0.19 g) was obtained as a white solid.

¹H NMR (300 MHz, CDCl₃) δ ppm 1.33 (t, J=7.5 Hz, 3H) 2.81 (q, J=7.5 Hz, 2H) 3.15-3.30 (m, 2H) 3.69 (m, 1H) 3.89-3.93 (m, 2H) 4.02-4.18 (m, 4H) 6.64-6.71 (m, 2H) 7.32 (m, 1H) 8.98 (brs, 1H)

### Example 223

### 2-[(2-chloro-5-fluorophenoxy)methyl]-N-thieno[2,3-b]pyrazin-7-ylmorpholine-4-carboxamide

### (1) phenyl thieno[2,3-b]pyrazin-7-ylcarbamate

By reaction in the same manner as in Example 202(1) and using thieno[2,3-b]pyrazin-7-amine (0.60 g), the object product (0.63 g) was obtained as a white solid.

¹H NMR (300 MHz, CDCl₃) δ ppm 7.22-7.29 (m, 3H) 7.39-7.45 (m, 2H) 8.04 (s, 1H) 8.15 (brs, 1H) 8.59-8.61 (m, 2H)

### (2) 2-[(2-chloro-5-fluorophenoxy)methyl]-N-thieno[2,3-b]pyrazine-7-ylmorpholine-4-carboxamide

By reaction in the same manner as in Example 202(2) and using phenyl thieno[2,3-b]pyrazin-7-ylcarbamate (0.29 g) obtained in the above-mentioned reaction and 2-[(2-chloro-5-fluorophenoxy)methyl]morpholine hydrochloride (0.30 g) synthesized by a method similar to that in Example 18, the object product (0.26 g) was obtained as a white solid.

¹H NMR (300 MHz, CDCl₃) δ ppm 3.14-3.30 (m, 2H) 3.74 (m, 1H) 3.96-4.10 (m, 4H) 4.17-4.27 (m, 2H) 6.30-6.73 (m, 2H) 7.30 (dd, J=6.0, 8.7 Hz, 1H) 7.83 (brs, 1H) 8.09 (s, 1H) 8.53-8.57 (m, 2H)

### Example 224

### 4-[(2,5-dichlorophenoxy)methyl]-N-[1-(pyridin-2-ylmethyl)-1H-pyrazol-4-yl]pyridine-2-carboxamide

### (1) tert-butyl 4-[({4-[(2,5-dichlorophenoxy)methyl]pyridin-2-yl}carbonyl)amino]-1H-pyrazole-1-carboxylate

Using 4-[(2,5-dichlorophenoxy)methyl]pyridine-2-carboxylic acid (1.0 g) and tert-butyl 4-amino-1H-pyrazole-1-carboxylate (614 mg) and in the same manner as in Example 203(1), the object product (1.14 g, 74%) was obtained.

¹H NMR (300 MHz, DMSO-d₆) δ ppm 1.60 (s, 9H) 5.49 (s, 2H) 7.10 (dd, J=2.45, 8.48 Hz, 1H) 7.38 (d, J=2.26 Hz, 1H) 7.54 (d, J=8.48 Hz, 1H) 7.72 (dd, J=1.60, 4.99 Hz, 1H) 8.15 (s, 1H) 8.22 (s, 1H) 8.58 (s, 1H) 8.78 (d, J=5.09 Hz, 1H) 11.30 (s, 1H)

### (2) 4-[(2,5-dichlorophenoxy)methyl]-N-1H-pyrazol-4-ylpyridine-2-carboxamide dihydrochloride

In the same manner as in Example 203(2) and using tert-butyl 4-[({4-[(2,5-dichlorophenoxy)methyl]pyridin-2-yl}carbonyl)amino]-1H-pyrazole-1-carboxylate (1.14 g) obtained in the above-mentioned reaction, the object product (885 mg, 90%) was obtained.

¹H NMR (300 MHz, DMSO-d₆) δ ppm 5.50 (s, 2H) 7.10 (dd, J=1.89, 8.71 Hz, 1H) 7.39 (d, J=1.89 Hz, 1H) 7.54 (d, J=8.33 Hz, 1H) 7.73 (d, J=4.16 Hz, 1H) 8.08-8.34 (m, 3H) 8.78 (d, J=4.92 Hz, 1H) 9.51 (brs, 1H) 11.18 (s, 1H)

### (3) 4-[(2,5-dichlorophenoxy)methyl]-N-[1-(pyridin-2-ylmethyl)-1H-pyrazol-4-yl]pyridine-2-carboxamide

In the same manner as in Example 203(3) and using 4-[(2,5-dichlorophenoxy)methyl]-N-1H-pyrazol-4-ylpyridine-2-carboxamide dihydrochloride (400 mg) obtained in the above-mentioned reaction and 2-bromomethylpyridine hydrobromide (278 mg), the object product (710 mg) was obtained.

¹H NMR (300 MHz, DMSO-d₆) δ ppm 5.43 (s, 2H) 5.48 (s, 2H) 7.04 (d, J=7.72 Hz, 1H) 7.10 (dd, J=2.26, 8.48 Hz, 1H) 7.27-7.35 (m, 1H) 7.38 (d, J=2.26 Hz, 1H) 7.53 (d, J=8.48 Hz, 1H) 7.68 (dd, J=1.51, 4.90 Hz, 1H) 7.77 (td, J=7.72, 1.88 Hz, 1H) 7.83 (s, 1H) 8.21 (s, 1H) 8.29 (s, 1H) 8.54 (dd, J=0.85, 4.80 Hz, 1H) 8.75 (d, J=4.90 Hz, 1H) 11.03 (s, 1H)

### Example 225

### 3-{[(2-chlorophenyl)(methyl)amino]methyl}-N-[1-(4-fluorobenzyl)-1H-pyrazol-4-yl]benzamide

### (1) methyl 3-{[(2-chlorophenyl)(methyl)amino]methyl}benzoate

To a solution of 2-chloro-N-methylaniline (0.20 g) and methyl 3-formylbenzoate (0.23 g) in acetonitrile (4 mL) was added sodium triacetoxy borohydride (0.6 g), and the mixture was stirred at room temperature for 10 hr. Saturated aqueous sodium hydroxide solution was added to the residue, and the mixture was extracted with ethyl acetate. The organic layer was washed with saturated brine and dried over magnesium sulfate, and the solvent was evaporated under reduced pressure. The residue was purified by silica gel column to give the object product (0.10 g, 24%).

LC-MS: [M+H]⁺ 290.00

### (2) 3-{[(2-chlorophenyl)(methyl)amino]methyl}benzoic acid

By reaction in the same manner as in Example 201(2) and using methyl 3-{[(2-chlorophenyl)(methyl)amino]methyl}benzoate (0.10 g) obtained in the above-mentioned reaction, the object product (0.03 g, 31%) was obtained as a white solid.

LC-MS: [M+H]+ 276.08

### (3) 3-{[(2-chlorophenyl)(methyl)amino]methyl}-N-[1-(4-fluorobenzyl)-1H-pyrazol-4-yl]benzamide

By reaction in the same manner as in Example 203(1) and using 3-{[(2-chlorophenyl)(methyl)amino]methyl}benzoic acid (0.02 g) obtained in the above-mentioned reaction, 1-(4-fluorobenzyl)-1H-pyrazol-4-amine (0.02 g) and o-(benzotriazol-1-yl)-N,N,N,N-tetramethyluroniumtetrafluoroborate (0.05 mg), the object product (0.02 g, 30%) was obtained as a white solid.

¹H NMR (300 MHz, CDCl₃) δ ppm 3.01 (s, 3H) 4.50 (s, 2H) 5.28 (s, 2H) 7.02 (t, J=8.4 Hz, 2H) 7.08-7.15 (m, 1H) 7.17-7.28 (m, 3H) 7.31-7.45 (m, 3H) 7.76 (s, 1H) 8.05-8.11 (m, 2H) 8.67 (s, 1H)

### Experimental Example 1

### SCD Inhibitory Activity

### “Test Compound”

(1) compound of Example 59
(2) compound of Example 93
(3) compound of Example 99
(4) compound of Example 108
(5) compound of Example 113
(6) compound of Example 116
(7) compound of Example 118
(8) compound of Example 149
(9) compound of Example 167
(10) compound of Example 169
(11) compound of Example 201
(12) compound of Example 202
(13) compound of Example 212
(14) compound of Example 225

### “Test Method” Measurement of SCD Inhibitory Activity Using Microsome (TLC Detection System)

A test compound (10 mM) diluted with DMSO in advance was secondarily diluted to 3/1000 with 3× buffer (300 mmol/L NaH₂PO₄ [pH 7.4], 450 mM KCl, 30 mM NaF, 9 mM MgCl₂, 4.5 mM glutathione [reduced form], 0.3% BSA [fatty acid free, SIGMA]). The test compound (10 μL) diluted with the assay buffer was dispensed to a PP 96-deep well block, and a microsome fraction (10 μL) diluted with a microsome buffer was added thereto. The enzyme reaction was started by the addition of 10 μL of [¹⁴C] stearoyl-CoA (American Radiolabeled Chemicals [ARC], Inc.) diluted to 10 μCi/mL with 9 mmol/L NADH. For evaluation of the compound, an enzyme reaction using rat liver microsome (20 μg) was performed for 15 min. The reaction was quenched by the addition of 10 μL of 2.5N NaOH, a plate seal was applied, and the reaction mixture was incubated overnight in a dry heater set to 65° C. to allow saponification. Solvent extraction of fatty acid was based on the Bligh&Dyer method (1). Formic acid:methanol:chloroform (1:6:3) (200 μL) was added, the state of single layer was maintained, the mixture was sufficiently stirred, and pure water (120 μL) was added to allow separation into two layers. The lower chloroform layer (10 μL) was spotted on reversed-phase TLC plate (RP-18, 1154230001, Merck Japan, Ltd.) and developed with acetonitrile:pure water:acetic acid (95:4.5:0.5). The TLC plate was dried and transferred onto an Imaging Plate (Fuji Photo Film Co., Ltd.) for not less than 5 hr. For detection, BAS-5000 (Fuji Photo Film Co., Ltd) was used and the obtained spot images were converted to numerical values using Multi Gauge Ver2.3 (Fuji Photo Film Co., Ltd), based on which the SCD activity inhibitory rate (%) was determined.

### “Test Results”

TABLE 1
SCD activity inhibitory rate (%) by administration
of test compound (10 μM)

| | SCD activity | | SCD activity |
|---|---|---|---|
| | inhibitory | | inhibitory |
| Example No. | rate (%) | Example No. | rate (%) |
| 59 | 88 | 108 | 97 |
| 93 | 83 | 116 | 100 |
| 99 | 93 | 118 | 86 |
| 113 | 86 | 149 | 97 |
| 167 | 85 | 169 | 88 |
| 201 | 99 | 202 | 92 |
| 212 | 97 | 225 | 93 |

### Formulation Example 1

An SCD inhibitor containing compound (I) as an active ingredient (e.g., therapeutic agent for hypertriglyceridemia, hyperlipidemia etc.) can be produced, for example, according to the following formulations.

In the following formulations, as a component (additive) other than the active ingredient, those listed in the Japanese Pharmacopoeia, the Japanese Pharmacopoeia Japanese Pharmaceutical Codex or Japanese Pharmaceutical Excipients and the like can be used.

1. capsule

| | |
|---|---|
| (1) compound obtained in Example 1 | 10 mg |
| (2) lactose | 90 mg |
| (3) microcrystalline cellulose | 70 mg |
| (4) magnesium stearate | 10 mg |
| 1 capsule | 180 mg |

(1), (2), (3) and ½ of (4) are blended and granulated. The rest of (4) is added and the whole mixture is sealed in a gelatin capsule.

2. tablet

| | | |
|---|---|---|
| (1) compound obtained in Example 2 | 10 | mg |
| (2) lactose | 35 | mg |
| (3) cornstarch | 150 | mg |
| (4) microcrystalline cellulose | 30 | mg |
| (5) magnesium stearate | 5 | mg |
| 1 tablet | 230 | mg |

(1), (2), (3), ⅔ of (4) and ½ of (5) are blended and granulated. The rest of (4) and (5) is added to the granules and the mixture is press-molded into a tablet.

### INDUSTRIAL APPLICABILITY

Since compound (I) shows an SCD inhibitory action, the compound is highly useful as an agent for the prophylaxis and/or treatment of hypertriglyceridemia, hyperlipidemia (particularly hypertriglyceridemia) and the like.

This application is based on a patent application No. 2006-280625 filed in Japan, the contents of which are encompassed in full in the present specification. In addition, the patent documents and non-patent documents cited in the present specification are hereby incorporated in their entireties by reference, to the extent that they have been disclosed in the present specification.

1. An SCD inhibitor comprising a compound represented by the formula [I] 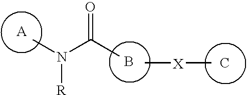
wherein
ring A is an optionally substituted aromatic ring,
ring B is an optionally substituted ring,
ring C is an optionally substituted aromatic ring,
R is a hydrogen atom, an optionally substituted hydrocarbon group or an optionally substituted heterocyclic group, and
X is a spacer having 1 to 5 atoms in the main chain,
or a salt thereof, or a prodrug thereof.

2. The agent of claim 1, wherein the ring A is an optionally substituted aromatic cyclic hydrocarbon or an optionally substituted 5- or 6-membered monocyclic aromatic heterocycle.

3. The agent of claim 1, wherein the ring B is an optionally substituted aromatic cyclic hydrocarbon or an optionally substituted 5- or 6-membered nitrogen-containing heterocycle.

4. The agent of claim 1, wherein the ring C is an optionally substituted 6-membered aromatic ring.

5. The agent of claim 1, wherein X is the formula —(CH₂)ₘ—Y—(CH₂)ₙ—
wherein m and n are each an integer of 0 to 4 (total of m and n does not exceed 4), and Y is a bond (when Y is a bond, m is not 0), —O—, —S—, —S(O)—, —S(O)₂— or —N(R¹)— (wherein R¹ is a hydrogen atom, an optionally substituted hydrocarbon group or an optionally substituted heterocyclic group).

6. The agent of claim 1, wherein X is —CH₂—O—.

7. The agent of claim 1, wherein R is a hydrogen atom.

8. The agent of claim 1, which is an agent for the prophylaxis or treatment of hyperlipidemia.

9. The agent of claim 8, which further comprises a drug having a blood lipid improving effect.

10. The agent of claim 1, which is an agent for the prophylaxis or treatment of diabetes or obesity.

11. A method for the prophylaxis or treatment of hyperlipidemia or obesity in a mammal, which comprises administering the agent of claim 1 to the mammal.

12. A method for the prophylaxis or treatment of diabetes or obesity in a mammal, which comprises administering the agent of claim 1 to the mammal.

13-14. (canceled)

15. A compound represented by the formula [II] 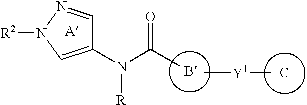
wherein
ring A′ is a further optionally substituted pyrazole ring,
ring B′ is an optionally substituted ring,
R² is an optionally substituted hydrocarbon group or an optionally substituted heterocyclic group,
ring C is an optionally substituted aromatic ring,
R is a hydrogen atom, an optionally substituted hydrocarbon group or an optionally substituted heterocyclic group, and
Y¹ is —C(R³)(R⁴)—X¹—
(R³ and R⁴ are each independently a hydrogen atom, an optionally substituted hydrocarbon group, an optionally substituted heterocyclic group, an optionally substituted hydroxy group, an optionally substituted amino group, an optionally substituted mercapto group, a cyano group, a nitro group, an optionally substituted acyl group or a halogen atom, and X¹ is a spacer having 1 to 4 atoms in the main chain,
provided that ring B′ is not a furan ring, R² is not a methyl group, and one of ring B′ and ring C is a heterocycle,
or a salt thereof.

16. The compound of claim 15, wherein ring A′ is a pyrazole ring.

17. The compound of claim 15, wherein ring B′ is benzene, piperidine, morpholine, pyrrolidine or pyridine.

18. The compound of claim 15, wherein R² is an optionally substituted C₁₋₆ alkyl group, an optionally substituted C₇₋₁₂ aralkyl group, optionally substituted C₆₋₁₀ aryl group or an optionally substituted 5- or 6-membered nitrogen-containing heterocyclic group.

19. The compound of claim 15, wherein ring C is an optionally substituted 6-membered aromatic ring.

20. The compound of claim 15, wherein R is a hydrogen atom.

21. The compound of claim 15, wherein Y¹ is —CH₂O—, —CH₂CH₂— or —CH₂CH₂O—.

22. 3-[(2-Chlorophenoxy)methyl]-N-[1-(4-fluorobenzyl)-1H-pyrazol-4-yl]pyrrolidine-1-carboxamide;
4-[(2-chlorophenoxy)methyl]-N-[1-(2-hydroxyethyl)-1H-pyrazol-4-yl]pyridine-2-carboxamide;
4-[(2-chloro-5-fluorophenoxy)methyl]-N-[1-(4-fluorobenzyl)-1H-pyrazol-4-yl]pyridine-2-carboxamide;
N-[1-(4-fluorobenzyl)-1H-pyrazol-4-yl]-4-{[2-fluoro-5-(trifluoromethyl)phenoxy]methyl}pyridine-2-carboxamide;
4-[(2,5-dichlorophenoxy)methyl]-N-[1-(pyridin-2-ylmethyl)-1H-pyrazol-4-yl]pyridine-2-carboxamide;
or a salt thereof.

23. A prodrug of the compound of claim 15.

24. A pharmaceutical agent comprising the compound of claim 15 or a prodrug thereof.

25. The pharmaceutical agent of claim 24, which is an SCD inhibitor.

26. The pharmaceutical agent of claim 24, which is an agent for the prophylaxis or treatment of hyperlipidemia.

27. The pharmaceutical agent of claim 26, further comprising a drug having a blood lipid improving effect.

28. The pharmaceutical agent of claim 24, which is an agent for the prophylaxis or treatment of diabetes or obesity.

29. A method for the prophylaxis or treatment of hyperlipidemia in a mammal, which comprises administering the compound of claim 15 or a prodrug thereof to the mammal.

30. A method for the prophylaxis or treatment of diabetes or obesity in a mammal, which comprises administering the compound of claim 15 or a prodrug thereof to the mammal.

31-32. (canceled)

33. A compound represented by the formula [III] 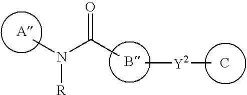
wherein
ring A″ is an optionally substituted aromatic heterocycle, 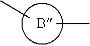
is a ring selected from 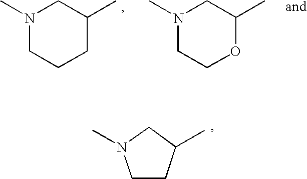
each of which is optionally substituted,
ring C is an optionally substituted aromatic ring,
R is a hydrogen atom, an optionally substituted hydrocarbon group or an optionally substituted heterocyclic group,
Y² is —C(R³)(R⁴)—X²—
wherein R³ and R⁴ are each independently a hydrogen atom, an optionally substituted hydrocarbon group, an optionally substituted heterocyclic group, an optionally substituted hydroxy group, an optionally substituted amino group, an optionally substituted mercapto group, a cyano group, a nitro group, an optionally substituted acyl group or a halogen atom, and
X² is a spacer having 1 to 4 atoms in the main chain,
ring A″ is not pyrazol-4-yl having a substituent at the 1-position, 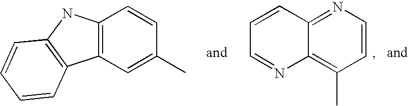
X² is not —NH—,
or a salt thereof.

34. The compound of claim 33, wherein ring A″ is an optionally substituted 5- or 6-membered nitrogen-containing aromatic heterocycle.

35. The compound of claim 33, wherein ring B″ is unsubstituted.

36. The compound of claim 33, wherein ring C is an optionally substituted 6-membered aromatic ring.

37. The compound of claim 33, wherein R is a hydrogen atom.

38. The compound of claim 33, wherein Y² is —CH₂O—, —CH₂CH₂— or —CH₂CH₂O—.

39. N-(4,6-Dimethylpyridin-2-yl)-2-[(2-fluorophenoxy)methyl]morpholine-4-carboxamide;
N-(4,6-dimethylpyridin-2-yl)-2-{[2-(trifluoromethyl)phenoxy]methyl}morpholine-4-carboxamide;
2-[(2-chloro-5-fluorophenoxy)methyl]-N-(4,6-dimethylpyridin-2-yl)morpholine-4-carboxamide;
2-[(2-chloro-5-fluorophenoxy)methyl]-N-(6-ethylpyridin-2-yl)morpholine-4-carboxamide;
2-[(2-chloro-5-fluorophenoxy)methyl]-N-[5-(hydroxymethyl)pyridin-2-yl]morpholine-4-carboxamide;
or a salt thereof.

40. A prodrug of the compound of claim 33.

41. A pharmaceutical agent comprising the compound of claim 33 or a prodrug thereof.

42. The pharmaceutical agent of claim 41, which is an SCD inhibitor.

43. The pharmaceutical agent of claim 41, which is an agent for the prophylaxis or treatment of hyperlipidemia.

44. The pharmaceutical agent of claim 43, further comprising a drug having a blood lipid improving effect.

45. The pharmaceutical agent of claim 41, which is an agent for the prophylaxis or treatment of diabetes or obesity.

46. A method for the prophylaxis or treatment of hyperlipidemia in a mammal, which comprises administering the compound of claim 33 or a prodrug thereof to the mammal.

47. A method for the prophylaxis or treatment of diabetes or obesity in a mammal, which comprises administering the compound of claim 33 or a prodrug thereof to the mammal.

48-49. (canceled)
